SEQUENCE LISTING (45 sequences)

SEQ ID NO: 1 - protein, 123 aa, Artificial sequence
  Synthetic F1 VH amino acid sequence
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 2 - protein, 107 aa, Artificial sequence
  Synthetic F1 VL amino acid sequence
EIVMTQSPSSVSASVGDRVAITCRASQGISSWLAWYQQKPGKAPKLLIYAASSLQSGVPSRFSGSGSGTDFTLTISSLQPEDFATYYCQQSYSTPLTFGGGTKVDIK

SEQ ID NO: 3 - protein, 123 aa, Artificial sequence
  Synthetic F8 VH amino acid sequence
EVQLLESGGGVVQHGRSLRLSCAASGFTFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 4 - protein, 107 aa, Artificial sequence; identical to SEQ ID NO: 2
  Synthetic F8 VL amino acid sequence

SEQ ID NO: 5 - protein, 122 aa, Artificial sequence
  Synthetic F9 VH amino acid sequence
EVQLLESGGGVVQLGGPDSPVQPLDSPFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 6 - protein, 107 aa, Artificial sequence; identical to SEQ ID NO: 2
  Synthetic F9 VL amino acid sequence

SEQ ID NO: 7 - protein, 123 aa, Artificial sequence
  Synthetic F26 VH amino acid sequence
EVQLLESGGGVVQRGRSLRLSCAASGFTFSSYGMHWVRQAPGKGLEWVAVISYDGSNKYYADSVKGRFTISRDNSKNTLYLPMNSLRAEDTAVYYCARPRAYYGAYGSSFDYWGQGTQVTVSS

SEQ ID NO: 8 - protein, 107 aa, Artificial sequence; identical to SEQ ID NO: 2
  Synthetic F26 VL amino acid sequence

SEQ ID NO: 9 - protein, 123 aa, Artificial sequence
  Synthetic F40 VH amino acid sequence
EVQLLESGGGVVQPGRSLRLSCAASGFTFSSYAMHWVRQAPGKGLEWVAVISYDGSNKYYADSVKGRFIISRDNSKNTVYLQMNSLRAEDTAVYYCARPTYVFTYTGSSFDYWGQGTQVTVSS

SEQ ID NO: 10 - protein, 107 aa, Artificial sequence
  Synthetic F40 VL amino acid sequence
DIQVTQSPSSLSASLGDTVSITCRASRGLTDSVAWYQQKPGQAPKLLIYAASTLQSGVPSRFGGSGSGSYFTLTITSLQPEDVATYYCQNYKSAPWTFGQGTKVEIK

SEQ ID NO: 11 - protein, 123 aa, Artificial sequence
  Synthetic F48 VH amino acid sequence
EVQLLESGGGVVQPGRSLRLSCAASGFTFSSYGMHWVRQAPGKGLEWVAVISYDGSNKYYADSVKGRFTISRDNSKNTLYLHMNSLRAEDTAVYYCARPRAYYGAYGSSFDYWGQGTQVTVSS

SEQ ID NO: 12 - protein, 107 aa, Artificial sequence; identical to SEQ ID NO: 2
  Synthetic F48 VL amino acid sequence

SEQ ID NO: 13 - protein, 123 aa, Artificial sequence
  Synthetic F50 VH amino acid sequence
EVQLLESGGGVVQRGRSLRLSCAASGFTFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 14 - protein, 107 aa, Artificial sequence; identical to SEQ ID NO: 2
  Synthetic F50 VL amino acid sequence

SEQ ID NO: 15 - protein, 123 aa, Artificial sequence
  Synthetic F100 VH amino acid sequence
EVQLLESGGGVVQPGRSLRLSCAASGFTFSSYGMHWVRQAPGKGLEWVAVISYDGSNKYYADSVKGRFTISRPNSKNTLYLQMNSLRAEDTAVYYCARPRAYYGAYGSSFDYWGQGTQVTVSS

SEQ ID NO: 16 - protein, 107 aa, Artificial sequence; identical to SEQ ID NO: 2
  Synthetic F100 VL amino acid sequence

SEQ ID NO: 17 - protein, 123 aa, Artificial sequence
  Synthetic F112 VH amino acid sequence
EVQLLESGGGVVQPGRSLRLSCAASGFTFSSYGMHWVRQAPGKGLEWVAVISYDGSNKYYADSVKGRFTISRHNSKNTLYLQMNSLRAEDTAVYYCARPRAYYGAYGSSFDYWGQGTQVTVSS

SEQ ID NO: 18 - protein, 107 aa, Artificial sequence; identical to SEQ ID NO: 2
  Synthetic F112 VL amino acid sequence

SEQ ID NO: 19 - protein, 123 aa, Artificial sequence
  Synthetic F123 VH amino acid sequence
EVQLLESGGGVVQPERSLRLSCAASGFTFSSYGMHWVRQAPGKGLEWVAVISYDGSNKYYADSVKGRFTISRANSKNTLYLQMNSLRAEDTAVYYCARPRAYYGAYGSSFDYWGQGTQVTVSS

SEQ ID NO: 20 - protein, 107 aa, Artificial sequence; identical to SEQ ID NO: 2
  Synthetic F123 VL amino acid sequence

SEQ ID NO: 21 - protein, 123 aa, Artificial sequence
  Synthetic F131 VH amino acid sequence
EVQLLESGGGVVQPGRSLRLSCAASGFTFSSYGMHWVRQAPGKGLEWVAVISYDGSNKYYADSVKGRFTISRANSKNTLYLQMNSLRAEDTAVYYCARPRAYYGAYGSSFDYWGQGTQVTVSS

SEQ ID NO: 22 - protein, 107 aa, Artificial sequence; identical to SEQ ID NO: 2
  Synthetic F131 VL amino acid sequence

SEQ ID NO: 23 - protein, 123 aa, Artificial sequence
  Synthetic F138 VH amino acid sequence
EVQLLESGGGVVQPGRSLRLSCAASGFTFSSYGMHWVRQAPGKGLEWVAVISYDGSNKYYADSVKGRFTISTHNSKNTLYLQMNSLRAEDTAVYYCARPRAYYGAYGSSFDYWGQGTQVTVSS

SEQ ID NO: 24 - protein, 107 aa, Artificial sequence; identical to SEQ ID NO: 2
  Synthetic F138 VL amino acid sequence

SEQ ID NO: 25 - protein, 5 aa, Artificial sequence
  Synthetic HCDR1 amino acid sequence
SYGMH

SEQ ID NO: 26 - protein, 17 aa, Artificial sequence
  Synthetic HCDR2 amino acid sequence
VISYDGSNKYYADSVKG

SEQ ID NO: 27 - protein, 14 aa, Artificial sequence
  Synthetic HCDR3 amino acid sequence
PRAYYGAYGSSFDY

SEQ ID NO: 28 - protein, 11 aa, Artificial sequence
  Synthetic LCDR1 amino acid sequence
RASQGISSWLA

SEQ ID NO: 29 - protein, 7 aa, Artificial sequence
  Synthetic LCDR2 amino acid sequence
AASSLQS

SEQ ID NO: 30 - protein, 9 aa, Artificial sequence
  Synthetic LCDR3 amino acid sequence
QQSYSTPLT

SEQ ID NO: 31 - protein, 5 aa, Artificial sequence
  Synthetic HCDR1 amino acid sequence
SYAMH

SEQ ID NO: 32 - protein, 14 aa, Artificial sequence
  Synthetic HCDR3 amino acid sequence
PTYVFTYTGSSFDY

SEQ ID NO: 33 - protein, 11 aa, Artificial sequence
  Synthetic LCDR1 amino acid sequence
RASRGLTDSVA

SEQ ID NO: 34 - protein, 7 aa, Artificial sequence
  Synthetic LCDR2 amino acid sequence
AASTLQS

SEQ ID NO: 35 - protein, 8 aa, Artificial sequence
  Synthetic LCDR3 amino acid sequence
QNYKSAPW

SEQ ID NO: 36 - protein, 118 aa, Artificial sequence
  Synthetic huFR107 VH amino acid sequence
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 37 - protein, 111 aa, Artificial sequence
  Synthetic huFR107 VL amino acid sequence
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 38 - protein, 5 aa, Artificial sequence
  Gly Ser linker
GGGGS

SEQ ID NO: 39 - protein, 330 aa, Artificial sequence [sequence omitted]

SEQ ID NO: 40 - protein, 107 aa, Artificial sequence [sequence omitted]

SEQ ID NO: 41 - protein, 6 aa, Artificial sequence
  Synthetic hexa-histidine
HHHHHH

SEQ ID NO: 42 - protein, 4 aa, Artificial sequence
  Synthetic linker sequence
GFLG

SEQ ID NO: 43 - protein, 4 aa, Artificial sequence
  Synthetic linker sequence
GGFG

SEQ ID NO: 44 - protein, 5 aa, Artificial sequence
  Synthetic recognition motif
  misc_feature (3)..(3): Xaa can be any naturally occurring amino acid
LPXTG

SEQ ID NO: 45 - protein, 4 aa, Artificial sequence
  cathepsin-B-cleavable peptides
ALAL

(19) United States
(12) Patent Application Publication
(10) Pub. No.: US 2024/0209080 A1
(43) Pub. Date: Jun. 27, 2024

(54) FOLR1 BINDING AGENTS, CONJUGATES THEREOF AND METHODS OF USING THE SAME

(71) Applicant: PROFOUNDBIO US CO., Woodinville, WA (US)

(72) Inventor: Baiteng ZHAO, Bellevue, WA (US)

(21) Appl. No.: 18/554,745

(22) PCT Filed: Apr. 8, 2022
(86) PCT No.: PCT/US2022/023969

§ 371 Date: Oct. 10, 2023

Related U.S. Application Data
(60) Provisional application No. 63/173,406, filed on Apr. 10, 2021.

(51) Int. Cl. C07K 16/28; A61K 47/68; A61K 51/10; A61P 35/00

(52) U.S. Cl. CPC C07K 16/28; A61K 47/6803; A61K 47/6849; A61K 47/6889; A61K 51/1096; A61P 35/00; C07K 2317/31

(57) ABSTRACT

The present invention provides FOLR1 antibodies, antigen binding portions thereof, other binding agents and FOLR1 conjugates thereof for use in the treatment of cancer.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 760270_404 WO_SEQUENCE_LISTING.txt. The text file is 35.8 KB, was created on Apr. 5, 2022, and is being submitted electronically via EFS-Web.

### BACKGROUND

Folate Receptor 1 (FOLR1), also known as Folate Receptor-alpha, or Folate Binding Protein, is an N-glycosylated protein expressed on plasma membrane of cells. FOLR1 has a high affinity for folic acid and for several reduced folic acid derivatives. FOLR1 mediates delivery of the physiological folate, 5-methyltetrahydrofolate, to the interior of cells. FOLR1 is overexpressed in vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid (Weitman S D, et al., Cancer Res 52: 3396-3401 (1992); Antony A C, Annu Rev Nutr 16: 501-521 (1996); Kalli K R, et al. Gynecol Oncol 108: 619-626 (2008)). This expression pattern of FOLR1 makes it a desirable target for FOLR1-directed cancer therapy.

Although FOLR1 is present on a variety of types of cancer, clinical trials with FOLR1 antibodies and FOLR1 antibody drug conjugates have met with limited success. The present invention solves this and other needs.

### SUMMARY OF THE INVENTION

Provided herein are FOLR1 antibodies, antigen binding portions thereof and other binding agents as well as conjugates of such antibodies, antigen binding portions and other binding agents. Also provided are methods of using the FOLR1 antibodies, antigen binding portions and other binding agents and conjugates thereof for the treatment of cancer and other diseases. The invention disclosed herein is based in part on FOLR1 antibodies, antigen-binding portions thereof and other binding agents as well as conjugates thereof that specifically bind to FOLR1 and that exhibit improved properties. FOLR1 is an important and advantageous therapeutic target for the treatment of certain cancers. The FOLR1 antibodies, antigen binding portions thereof, other binding agents and conjugates thereof provide compositions and methods based on the use of such antibodies, antigen binding portions and related binding agents, and conjugates thereof, in the treatment of FOLR1+ cancers and other diseases.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having amino acids sequences selected from the sets of amino acid sequences set forth in the group consisting of: SEQ ID NO:25, SEQ ID NO:26, SEQ ID NO:27, SEQ ID NO:28, SEQ ID NO:29 and SEQ ID NO:30, respectively; and SEQ ID NO:31, SEQ ID NO:26, SEQ ID NO:32, SEQ ID NO:33, SEQ ID NO:34 and SEQ ID NO:35, respectively. In some embodiments, the VH and VL CDRs have the amino acids sequences set forth in SEQ ID NO:25, SEQ ID NO:26, SEQ ID NO:27, SEQ ID NO:28, SEQ ID NO:29 and SEQ ID NO:30, respectively. In some embodiments, the framework regions are human framework regions.

In some embodiments, the VH and VL regions have amino acid sequences that are selected from the pairs of amino acid sequences set forth in the group consisting of: SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14; respectively; SEQ ID NO:15 and SEQ ID NO: 16; respectively; SEQ ID NO: 17 and SEQ ID NO: 18; respectively; SEQ ID NO: 19 and SEQ ID NO:20; respectively; SEQ ID NO:21 and SEQ ID NO:22; respectively; and SEQ ID NO:23 and SEQ ID NO:24; respectively; wherein the heavy and light chain framework regions are optionally modified with from 1 to 8 amino acid substitutions, deletions or insertions in the framework regions.

In some embodiments, the VH and VL regions have amino acid sequences that are selected from the pairs of amino acid sequences set forth in the group consisting of: SEQ ID NO:1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14; respectively; SEQ ID NO: 15 and SEQ ID NO: 16; respectively; SEQ ID NO: 17 and SEQ ID NO: 18; respectively; SEQ ID NO: 19 and SEQ ID NO:20; respectively; SEQ ID NO:21 and SEQ ID NO:22; respectively; and SEQ ID NO:23 and SEQ ID NO:24; respectively.

In some embodiments, the VH and VL regions have amino acid sequences that are selected from the pairs of amino acid sequences set forth in the group consisting of: SEQ ID NO:3 and SEQ ID NO:4, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 15 and SEQ ID NO: 16; respectively; SEQ ID NO: 17 and SEQ ID NO: 18; respectively; SEQ ID NO: 19 and SEQ ID NO:20; respectively; and SEQ ID NO:21 and SEQ ID NO:22; respectively.

In some embodiments, the VH and VL regions have amino acid sequences that are selected from the pairs of amino acid sequences set forth in the group consisting of: SEQ ID NO:3 and SEQ ID NO:4, respectively; SEQ ID NO:7 and SEQ ID NO:8, respectively; and SEQ ID NO:21 and SEQ ID NO:22; respectively. In some embodiments, the VH and VL regions have amino acid sequences that are set forth in SEQ ID NO:3 and SEQ ID NO:4, respectively. In some embodiments, the VH and VL regions have amino acid sequences that are set forth in SEQ ID NO:7 and SEQ ID NO:8, respectively. In some embodiments, the VH and VL regions have amino acid sequences that are set forth in SEQ ID NO:21 and SEQ ID NO:22, respectively.

In some embodiments, the binding agent is an antibody or an antigen-binding portion thereof. In some embodiments, the binding agent is a monoclonal antibody, a Fab, a Fab′, an F(ab′), an Fv, a scFv, a single domain antibody, a diabody, a bi-specific antibody, or a multi-specific antibody. In some embodiments, the heavy chain variable region further comprises a heavy chain constant region. In some embodiments, the heavy chain constant region is of the IgG isotype. In some embodiments, the heavy chain constant region is an IgG1 constant region. In some embodiments, the IgG1 constant region has the amino acid sequence set forth in SEQ ID NO:39. In some embodiments, the heavy chain constant region is an IgG4 constant region. In some embodiments, the heavy chain constant region further comprises at least amino acid modification that decreases binding affinity to human FcgammaRIII. In some embodiments, the light chain variable region further comprises a light chain constant region. In some embodiments, the light chain constant region is of the kappa isotype. In some embodiments, the light chain constant region has the amino acid sequence set forth in SEQ ID NO:40.

In some embodiments, the binding agent is mono-specific. In some embodiments, the binding agent is bivalent. In some embodiments, the binding agent is bispecific.

In some embodiments, provided is a pharmaceutical composition comprising any of the binding agents described herein and a pharmaceutically acceptable carrier. In some embodiments, provided is a nucleic acid encoding any of the binding agents described herein. In some embodiments, provided is a vector comprising any of the nucleic acids encoding any of the binding agents described herein. In some embodiments, provided is a cell line comprising any of the vectors encoding any of the binding agents as described herein or any of the nucleic acids encoding any of the binding agents as described herein.

In some embodiments, provided is a conjugate comprising any of the binding agents as described herein, at least one linker attached to the binding agent; and at least one drug attached to each linker. In some embodiments, each drug is selected from a cytotoxic agent, an immunomodulatory agent, a nucleic acid, a growth inhibitory agent, a PROTAC, a toxin and a radioactive isotope. In some embodiments, each linker is attached to the binding agent via an interchain disulfide residue, a lysine residue, an engineered cysteine residue, a glycan, a modified glycan, an N-terminal residue of the binding agent or a polyhistidine peptide attached to the binding agent. In some embodiments, the average drug loading of the conjugate is from about 1 to about 8, about 2, about 4, about 6, about 8, about 10, about 12, about 14, about 16, about 3 to about 5, about 6 to about 8 or about 8 to about 16.

In some embodiments of a conjugate, the drug is a cytotoxic agent. In some embodiments, the cytotoxic agent is selected from the group consisting of an auristatin, a maytansinoid, a camptothecin, a duocarmycin or a calicheamicin. In some embodiments, the cytotoxic agent is an auristatin. In some embodiments, the cytotoxic agent is MMAE or MMAF. In some embodiments, the cytotoxic agent is a camptothecin. In some embodiments, the cytotoxic agent is exatecan. In some embodiments, the cytotoxic agent is SN-38. In some embodiments, the cytotoxic agent is a calicheamicin. In some embodiments, the cytotoxic agent is a maytansinoid. In some embodiments, the maytansinoid is maytansine, maytansinol or a maytansine analog in DM1, DM3 and DM4, or ansamatocin-2.

In some embodiments, the linker comprises mc-VC-PAB, CL2, CL2A or (Succinimid-3-yl-N)—(CH2)n-C(═O)-Gly-Gly-Phe-Gly-NH—CH2-O—CH2-(C═O)—, wherein n=1 to 5. In some embodiments, the linker comprises mc-VC-PAB. In some embodiments, the linker comprises CL2A. In some embodiments, the linker comprises CL2. In some embodiments, the linker comprises (Succinimid-3-yl-N)—(CH2)n-C(═O)-Gly-Gly-Phe-Gly-NH—CH2-O—CH2-(C═O)—. The conjugate of claim 43, wherein the linker is attached to at least one molecule of exatecan. In some embodiments,

In some embodiments, the drug is an immune modulatory agent. In some embodiments, the immune modulatory agent is selected from the group consisting of a TRL7 agonist, a TLR8 agonist, a STING agonist, or a RIG-I agonist. In some embodiments, the immune modulatory agent is an TLR7 agonist. In some embodiments, the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the immune modulatory agent is a TLR8 agonist. In some embodiments, the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, the immune modulatory agent is a STING agonist. In some embodiments, the immune modulatory agent is a RIG-I agonist. In some embodiments, the RIG-I agonist is selected from KIN1148, SB-9200, KIN700, KIN600, KIN500, KIN100, KIN101, KIN400 and KIN2000. In some embodiments, wherein the linker is selected from the group consisting of mc-VC-PAB, CL2, CL2A and (Succinimid-3-yl-N)—(CH2)n-C(═O)-Gly-Gly-Phe-Gly-NH—CH2-O—CH2-(C═O)—, wherein n=1 to 5.

In some embodiments, provided is a pharmaceutical composition comprising any of the conjugate described herein and a pharmaceutically acceptable carrier.

In some embodiments, provided is a method of treating a FOLR1+ cancer, comprising administering to a subject in need thereof a therapeutically effective amount any of the binding agents described herein, any of the conjugates described herein or any of the pharmaceutical compositions of binding agents or conjugates described herein. In some embodiments, the FOLR1+ cancer is a solid tumor. In some embodiments, the FOLR1+ cancer is selected from lung cancer, non-small cell lung cancer, ovarian cancer, breast cancer, uterine cancer, cervical cancer, endometrial cancer, pancreatic cancer, and renal cell cancer. In some embodiments,

In some embodiments, the method further comprises administering an immunotherapy to the subject. In some embodiments, the immunotherapy comprises a checkpoint inhibitor. In some embodiments, the checkpoint inhibitor is selected from an antibody that specifically binds to human PD-1, human PD-L1, or human CTLA4. In some embodiments, the checkpoint inhibitor is pembrolizumab, nivolumab, cemiplimab or ipilimumab. In some embodiments, the method further comprises administering chemotherapy to the subject.

In some embodiments, the method comprises administering any of the conjugates described herein or any of the pharmaceutical compositions described herein to the subject. In some embodiments, the binding agent, conjugate or pharmaceutical composition is administered intravenously. In some embodiments, the binding agent, conjugate or pharmaceutical composition is administered in a dose of about 0.1 mg/kg to about 12 mg/kg.

In some embodiments of the method, a treatment outcome of the subject is improved. In some embodiments, the improved treatment outcome is an objective response selected from stable disease, a partial response or a complete response. In some embodiments, the improved treatment outcome is reduced tumor burden. In some embodiments, the improved treatment outcome is progression-free survival or disease-free survival.

In some embodiments, provided is the use of any of the binding agents described herein or any of the pharmaceutical compositions of binding agents described herein for the treatment of FOLR1+ cancer in a subject. In some embodiments, provided is the use of any of the conjugates described herein or any of the pharmaceutical compositions described herein for the treatment of FOLR1+ cancer in a subject.

These and other aspects of the present invention may be more fully understood by reference to the following detailed description, non-limiting examples of specific embodiments and the appended drawings.

### FIGURES

FIG. 1. Comparison of anti-FOLR1 antibody binding to Hela cells.

FIG. 2. Comparison of anti-FOLR1 antibody binding ability to RPTEC/TERT1 cells.

FIG. 3. Dose-dependent binding of anti-FOLR1 antibodies to Hela cells.

FIG. 4. Dose-dependent binding of anti-FOLR1 antibodies to RPTEC/TERT1 cells.

FIG. 5. Internalization of anti-FOLR1 antibodies into Hela cells.

FIG. 6. Internalization of anti-FOLR1 antibodies into RPTEC/TERT1 cells.

FIG. 7. Comparison of anti-FOLR-1 conjugate binding to the target FOLR1 protein.

FIG. 8. Comparison of anti-FOLR-1 conjugates binding to the target FLOR 1 protein.

FIG. 9. Comparison of anti-FOLR-1 conjugates binding to Hela cells.

FIG. 10. Comparison of an-huFOLR-1 conjugates binding to IGROV-1, OVCAR3 and OV90 cells.

FIG. 11. Comparison of anti-FOLR-1 conjugate internalization on Hela cells.

FIG. 12. Comparison of anti-FOLR-1 conjugate internalization on OVCAR-3 cells.

FIG. 13. Comparison of anti-FOLR-1 conjugates internalization on OV90 cells.

FIG. 14. Comparison of anti-FOLR-1 conjugates internalization on IGROV-1 cells.

FIG. 15. Comparison of anti-huFOLR-1 conjugate cytotoxicity on Hela cells.

FIG. 16. Comparison of anti-huFOLR-1 conjugate cytotoxicity on OV90 cells.

FIG. 17. Comparison of anti-huFOLR-1 conjugate cytotoxicity on OVCAR-3 cells.

FIG. 18. Comparison of anti-huFOLR-1 conjugate cytotoxicity on IGROV-1 cells.

FIG. 19. Pharmacokinetics of anti-FOLR-1 conjugates.

FIG. 20. Effects of the anti-FOLR-1 conjugates on body weight.

FIG. 21. F131 Binding assay on JEG-3 by FACS.

FIG. 22. F131 Binding assay on PC-3 by FACS.

FIG. 23. F131 Internalization in tumor cell lines.

FIG. 24. In vivo efficacy of F131 conjugates in CDX on OVCAR-3.

FIG. 25. In vivo efficacy of F131 conjugates in CDX on HCC827.

FIG. 26. In vivo efficacy of F131 conjugates in CDX on H441.

FIG. 27. In vivo efficacy of F131 conjugates in CDX on OVCAR-3.

FIG. 28. In vivo efficacy of F131 conjugates in CDX on KB.

FIG. 29. In vivo efficacy of F131 conjugates in CDX on HCC827.

FIG. 30. In vivo efficacy of F131 conjugates in CDX on H441.

FIG. 31. In vivo efficacy of F131 conjugates in CDX on OV90.

FIG. 32. In vivo efficacy of F131 conjugates in CDX on OVCAR-3.

FIG. 33. In vivo efficacy of F131 conjugates in CDX on KB.

FIG. 34. PK study in Rat model of F131 and conjugates.

FIG. 35. PK study in Rat model of F131 and conjugates.

FIG. 36. F131-deruxtecan tolerability in the pilot cynomolgus toxicity study.

FIG. 37. F131-deruxtecan tolerability in the pilot cynomolgus toxicity study.

FIG. 38. F131-deruxtecan PK in the pilot cynomolgus toxicity study.

### DEFINITIONS

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

FOLR1, or folate receptor alpha, is a cell surface protein that binds to folate and reduced folic acid derivatives and mediates delivery of 5-methyltetrahydrofolate and folate analogs into the interior of cells. It is also referred to as FR-alpha, Adult folate-binding protein, FBP, Folate receptor 1, Folate receptor-adult, KB cells FBP and Ovarian tumor-associated antigen MOv18. Human FOLR1 polypeptides include, but are not limited to, those having the amino acid sequence set forth in UniProt identifier P15328-1; this sequence is incorporated by reference herein.

As used herein, an “epitope” refers to the amino acids conventionally bound by an immunoglobulin VH/VL pair, such as the antibodies, antigen binding portions thereof and other binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target, such as human FOLR1, with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. The person of ordinary skill in the art can determine appropriate conditions under which the antibodies, antigen binding portions and other binding agents described herein selectively bind to FOLR1 using any suitable methods, such as titration of a binding agent in a suitable cell binding assay. A binding agent specifically bound to FOLR1 is not displaced by a non-similar competitor. In certain embodiments, a FOLR1 antibody or antigen-binding portion thereof or other binding agent is said to specifically bind to FOLR1 when it preferentially recognizes its target antigen, FOLR1, in a complex mixture of proteins and/or macromolecules.

In some embodiments, a FOLR1 antibody or antigen-binding portion thereof or other binding agent as described herein specifically binds to a FOLR1 polypeptide with a dissociation constant (KD or K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, a FOLR1 antibody or antigen-binding portion thereof or other binding agent as described herein specifically binds to a FOLR1 polypeptide with a dissociation constant (KD) of from about 10⁻⁵ M to 10⁻⁶ M. In some embodiments, a FOLR1 antibody or antigen-binding portion thereof or other binding agent as described herein specifically binds to a FOLR1 polypeptide with a dissociation constant (KD) of from about 10⁻⁶ M to 10⁻⁷ M. In some embodiments, a FOLR1 antibody or antigen-binding portion thereof or other binding agent as described herein specifically binds to a FOLR1 polypeptide with a dissociation constant (KD) of from about 10⁻⁷ M to 10⁻⁸ M. In some embodiments, a FOLR1 antibody or antigen-binding portion thereof or other binding agent as described herein specifically binds to a FOLR1 polypeptide with a dissociation constant (KD) of from about 10⁻⁸ M to 10⁻⁹ M. In some embodiments, a FOLR1 antibody or antigen-binding portion thereof or other binding agent as described herein specifically binds to a FOLR1 polypeptide with a dissociation constant (KD) of from about 10⁻⁹ M to 10⁻¹⁰ M. In some embodiments, a FOLR1 antibody or antigen-binding portion thereof or other binding agent as described herein specifically binds to a FOLR1 polypeptide with a dissociation constant (KD) of from about 10⁻¹⁰ M to 10⁻¹¹ M. In some embodiments, a FOLR1 antibody or antigen-binding portion thereof or other binding agent as described herein specifically binds to a FOLR1 polypeptide with a dissociation constant (KD) of from about 10⁻¹¹ M to 10⁻¹² M. In some embodiments, a FOLR1 antibody or antigen-binding portion thereof or other binding agent as described herein specifically binds to a FOLR1 polypeptide with a dissociation constant (KD) of less than 10⁻¹² M.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Other terms are defined herein within the description of the various aspects of the invention.

### DETAILED DESCRIPTION

Provided herein are FOLR1 binding antibodies (also referred to as FOLR1 antibodies) and antigen binding portions thereof and other binding agents that specifically bind to human FOLR1. Also provided herein are conjugates of the FOLR1 antibodies and antigen binding portions and other binding agents bound to drugs, such as cytotoxic agents or immune modulatory agents (also referred to as FOLR1 conjugates). In some embodiments, the FOLR1 antibodies, antigen binding portions, other binding agents and conjugates specifically bind to and reduce the number of FOLR1+ cells in a subject. In some embodiments, the FOLR1 antibodies, antigen binding portions, other binding agents and/or conjugates specifically bind to and reduce the number of FOLR1+ cancer cells in a subject. In some embodiments, the FOLR1 antibodies, antigen binding portions, other binding agents and/or conjugates specifically bind to and reduce the number of FOLR1+ cells associated with a disease or condition in a subject.

In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14; respectively; SEQ ID NO: 15 and SEQ ID NO: 16; respectively; SEQ ID NO: 17 and SEQ ID NO: 18; respectively; SEQ ID NO: 19 and SEQ ID NO:20; respectively; SEQ ID NO:21 and SEQ ID NO:22; respectively; and SEQ ID NO:23 and SEQ ID NO:24; respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO: 1 and SEQ ID NO:2, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO:3 and SEQ ID NO:4, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO:5 and SEQ ID NO:6, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO:7 and SEQ ID NO:8, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO:9 and SEQ ID NO: 10, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO: 11 and SEQ ID NO: 12; respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO: 13 and SEQ ID NO: 14, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO: 15 and SEQ ID NO: 16, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO: 17 and SEQ ID NO:18, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO: 19 and SEQ ID NO:20, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO:21 and SEQ ID NO:22, respectively. In some embodiments, the FOLR1 antibodies or antigen binding portions thereof comprise a heavy chain variable (VH) region and a light chain variable (VL) region, the VH and VL regions having the amino acid sequences set forth in SEQ ID NO:23 and SEQ ID NO:24, respectively.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO:18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO:14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. The phrase “wherein the CDRs of the heavy or light chain variable regions are not modified” refers to the VH and VL CDRs that do not have amino acid substitutions, deletions or insertions.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 1 and SEQ ID NO:2, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:1 and SEQ ID NO:2, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO:9 and SEQ ID NO: 10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:9 and SEQ ID NO: 10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 11 and SEQ ID NO: 12; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO: 11 and SEQ ID NO: 12; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 13 and SEQ ID NO: 14, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO: 13 and SEQ ID NO: 14, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 15 and SEQ ID NO: 16, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:15 and SEQ ID NO: 16, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 17 and SEQ ID NO: 18, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:17 and SEQ ID NO: 18, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 19 and SEQ ID NO:20, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO: 19 and SEQ ID NO:20, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO:21 and SEQ ID NO:22, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:21 and SEQ ID NO:22, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the FOLR1 antibodies or antigen binding portions thereof comprise a heavy chain variable (VH) region and a light chain variable (VL) region, the VH and VL regions having amino acid sequences set forth in the pairs of amino acid sequences selected from SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:23 and SEQ ID NO:24, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the binding agent specifically binds to FOLR1. In some embodiments, the binding agent comprises a heavy chain variable (VH) region and a light chain variable (VL) region, the VH and VL regions having the amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and

VL regions having the amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in the pairs of amino acid sequences selected from SEQ ID NO:1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, a binding agent includes a FOLR1 antibody or antigen binding portion(s) thereof and can include other peptides or polypeptides covalently attached to the FOLR1 antibody or antigen binding portion thereof. In any of these embodiments, the binding agent specifically binds to FOLR1.

In some embodiments, provided herein is a binding agent comprising a heavy chain variable (VH) region and a light chain variable (VL) region, the VH and VL regions having the amino acid sequences set forth in SEQ ID NO:1 and SEQ ID NO:2, respectively; wherein the binding agent specifically binds to FOLR1. In some sequences set forth SEQ ID NO: 1 and SEQ ID NO:2, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO: 1 and SEQ ID NO:2, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:3 and SEQ ID NO:4, respectively; wherein the binding agent specifically binds to FOLR1. In some embodiments, the binding agent comprises a heavy chain variable (VH) region and a light chain variable (VL) region, the VH and VL regions having the amino acid sequences set forth SEQ ID NO:3 and SEQ ID NO:4, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:5 and SEQ ID NO:6, respectively; wherein the binding agent specifically binds to FOLR1. In some embodiments, the binding agent comprises a heavy chain variable (VH) region and a light chain variable (VL) region, the VH and VL regions having the amino acid sequences set forth in SEQ ID NO:5 and SEQ ID NO:6, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:7 and SEQ ID NO:8, respectively; wherein the binding agent specifically binds to FOLR1. In some embodiments, the binding agent comprises a heavy chain variable (VH) region and a light chain variable (VL) region, the VH and VL regions having the amino acid sequences set forth in SEQ ID NO:7 and SEQ ID NO:8, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions the having amino acid sequences set forth in SEQ ID NO:9 and SEQ ID NO: 10, respectively; wherein the binding agent specifically binds to FOLR1. In some embodiments, the binding agent comprises a heavy chain variable (VH) region and a light chain variable (VL) region, the VH and VL regions having the amino acid sequences set forth in SEQ ID NO:9 and SEQ ID NO: 10, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO:9 and SEQ ID NO: 10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:9 and SEQ ID NO:10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 11 and SEQ ID NO: 12; respectively; wherein the binding agent specifically binds to FOLR1. In some sequences set forth in SEQ ID NO: 11 and SEQ ID NO: 12; respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO: 11 and SEQ ID NO: 12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 11 and SEQ ID NO: 12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 13 and SEQ ID NO: 14, respectively; wherein the binding agent specifically binds to FOLR1. In some embodiments, the binding agent comprises a heavy chain variable (VH) region and a light chain variable (VL) region, the VH and VL regions having the amino acid sequences set forth SEQ ID NO: 13 and SEQ ID NO: 14, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO: 13 and SEQ ID NO: 14, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 13 and SEQ ID NO: 14, respectively; wherein the heavy and light chain variable framework regions are optionally modified with from 1 to 8, 1 to 6, 1 to 4 or 1 to 2 the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 15 and SEQ ID NO: 16, respectively; wherein the binding agent specifically binds to FOLR1. In some embodiments, the binding agent comprises a heavy chain variable (VH) region and a light chain variable (VL) region, the VH and VL regions having the amino acid sequences set forth SEQ ID NO: 15 and SEQ ID NO: 16, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO: 15 and SEQ ID NO: 16, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 15 and SEQ ID NO: 16, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 17 and SEQ ID NO: 18, respectively; wherein the binding agent specifically binds to FOLR1. In some embodiments, the binding agent comprises a heavy chain variable (VH) region and a light chain variable (VL) region, the VH and VL regions having the amino acid sequences set forth SEQ ID NO: 17 and SEQ ID NO: 18, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 19 and SEQ ID NO:20, respectively; wherein the binding agent specifically binds to FOLR1. In some embodiments, the binding agent comprises a heavy chain variable (VH) region and a light chain variable (VL) region, the VH and VL regions having the amino acid sequences set forth SEQ ID NO: 19 and SEQ ID NO:20, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO: 19 and SEQ ID NO:20, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 19 and SEQ ID NO:20, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:21 and SEQ ID NO:22, respectively; wherein the binding agent specifically binds to FOLR1. In some sequences set forth SEQ ID NO:21 and SEQ ID NO:22, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO:21 and SEQ ID NO:22, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:21 and SEQ ID NO:22, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:23 and SEQ ID NO:24, respectively; wherein the binding agent specifically binds to FOLR1. In some embodiments, the binding agent comprises a heavy chain variable (VH) region and a light chain variable (VL) region, the VH and VL regions having the amino acid sequences set forth SEQ ID NO:23 and SEQ ID NO:24, respectively; wherein the binding agent specifically binds to FOLR1 with a higher binding affinity (lower Kd) than antibody FR107. In some embodiments, provided herein is a binding agent comprising a heavy chain variable (VH) region and a light chain variable (VL) region, the VH and VL regions having the amino acid sequences set forth in SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:23 and SEQ ID NO:24, respectively; wherein the heavy and light chain variable framework regions are optionally modified with from 1 to 8, 1 to 6, 1 to 4 or 1 to 2 the CDRs of the heavy or light chain variable regions are not modified.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in the sets of amino acid sequences selected from (i) SEQ ID NO:25, SEQ ID NO:26, SEQ ID NO:27, SEQ ID NO:28, SEQ ID NO:29 and SEQ ID NO:30, respectively; and (ii) SEQ ID NO:31, SEQ ID NO:26, SEQ ID NO:32, SEQ ID NO:33, SEQ ID NO:34 and SEQ ID NO:35, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in SEQ ID NO:25, SEQ ID NO:26, SEQ ID NO:27, SEQ ID NO:28, SEQ ID NO:29 and SEQ ID NO:30, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in SEQ ID NO:31, SEQ ID NO:26, SEQ ID NO:32, SEQ ID NO:33, SEQ ID NO:34 and SEQ ID NO:35, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in the sets of amino acid sequences selected from (i) SEQ ID NO:25, SEQ ID NO:26, SEQ ID NO:27, SEQ ID NO:28, SEQ ID NO:29 and SEQ ID NO:30, respectively; and (ii) SEQ ID NO:31, SEQ ID NO:26, SEQ ID NO:32, SEQ ID NO:33, SEQ ID NO:34 and SEQ ID NO:35, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in SEQ ID NO:25, SEQ ID NO:26, SEQ ID NO:27, SEQ ID NO:28, SEQ ID NO:29 and SEQ ID NO:30, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in SEQ ID NO:31, SEQ ID NO:26, SEQ ID NO:32, SEQ ID NO:33, SEQ ID NO:34 and SEQ ID NO:35, respectively. In some embodiments, each VH and VL region comprises a humanized framework region. In some embodiments, each VH and VL region comprises a human framework region.

In some embodiments, the compositions and methods described herein relate to reduction of FOLR1+ cells in a subject (e.g., reducing the number of FOLR1+ cells in a cancer or tumor) by a FOLR1 antibody, antigen binding portion thereof, other binding agent or conjugate thereof in vivo. In some embodiments, the compositions and methods described herein relate to the treatment of FOLR1+ cancer in a subject by administering a FOLR1 antibody, antigen binding portion thereof, other binding agent or conjugate thereof. In some embodiments, the compositions and methods described herein relate to the reduction in the number of FOLR1+ cells in a subject by administering a FOLR1 antibody, antigen binding portion thereof, other binding agent or conjugate thereof.

As used herein, the term “antibody” refers to immunoglobulin molecules and immunologically active portions of immunoglobulin molecules, i.e., molecules that contain an antigen binding site(s) that specifically binds to an antigen, e.g., human FOLR1. The term generally refers to antibodies comprised of two immunoglobulin heavy chain variable regions and two immunoglobulin light chain variable regions, including full length antibodies (having heavy and light chain constant regions).

Each heavy chain is composed of a variable region (abbreviated as VH) and a constant region. The heavy chain constant region may include three domains CH1, CH2 and CH3 and optionally a fourth domain, CH4. Each light chain is composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of a FOLR1 antibody refers to the portions of a FOLR1 antibody as described herein having the VH and VL sequences of the FOLR1 antibody or the CDRs of a FOLR1 antibody and that specifically binds to FOLR1. Examples of antigen binding portions include a Fab, a Fab′, a F(ab′)₂, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)₂ and Fv refer to the following: (i) a Fab fragment, i.e. a monovalent fragment composed of the VL, VH, CL and CH1 domains; (ii) a F(ab′)₂ fragment, i.e. a bivalent fragment comprising two Fab fragments linked to one another in the hinge region via a disulfide bridge; and (iii) a Fv fragment composed of the VL and VH domains, in each case of a FOLR1 antibody. Although the two domains of the Fv fragment, namely VL and VH, are encoded by separate coding regions, they may further be linked to one another using a synthetic linker, e.g. a poly-G4S amino acid sequence ((G4S)n′ disclosed as SEQ ID NO: 38, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antibody portion consisting of a single monomeric variable antibody domain. Single domains antibodies can be derived from the variable domain of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281: 161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the FOLR1 antibodies or antigen binding portions thereof are part of a bispecific or multispecific binding agent. Bispecific and multi-specific antibodies include the following: an scFv1-scFv2, an scFv12-Fc-scFv22, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2): 182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7): 1803-1812, 2014; and Bates and Power, Antibodies, 2019, 8, 28.

### Modification of VH and VL Regions

As to the VH and VL amino acid sequences, one of skill will recognize that individual substitutions, deletions or additions (insertions) to a nucleic acid encoding the VH or VL, or amino acids in polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to FOLR1.

In some embodiments, a conservatively modified variant of a FOLR1 antibody or antigen binding portion thereof can have an alteration(s) in the framework regions (FR); i.e., other than in the CDRs), e.g. a conservatively modified variant of a FOLR1 antibody has the amino acid sequences of the VH and VL CDRs (set forth in sets of amino acid sequences (i) SEQ ID NO:25, SEQ ID NO:26, SEQ ID NO:27, SEQ ID NO:28, SEQ ID NO:29 and SEQ ID NO:30, respectively; and (ii) SEQ ID NO:31, SEQ ID NO:26, SEQ ID NO:32, SEQ ID NO:33, SEQ ID NO:34 and SEQ ID NO:35, respectively) and has at least one conservative amino acid substitution in a framework region. In some embodiments, the VH and VL amino acid sequences collectively have no more than 8 or 6 or 4 or 2 or 1 conservative amino acid substitutions in the FR, as compared to the amino acid sequences of the unmodified VH and VL regions. In some embodiments, the VH and VL amino acid sequences have 8 to 1, 6 to 1, 4 to 1 or 2 to 1 conservative amino acid substitutions in the FR, as compared to the amino acid sequences of the unmodified VH and VL regions. In further aspects of any of these embodiments, a conservatively modified variant of the FOLR1 antibody, antigen binding portion thereof or other binding agent exhibits specific binding to FOLR1.

For conservative amino acid substitutions, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to FOLR1.

In some embodiments, a FOLR1 antibody or antigen binding portion thereof or other binding agent can be further optimized to, for example, decrease potential immunogenicity or optimize other functional property, while maintaining functional activity, for therapy in humans. In some embodiments, the FOLR1 antibodies or antigen binding portions thereof or other binding agents comprise a heavy chain variable (VH) region and a light chain variable (VL) region, the VH and VL regions having amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO:13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the FOLR1 antibodies or antigen binding portions thereof or other binding agents comprise a heavy chain variable (VH) region and a light chain variable (VL) region, the VH and VL regions having amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO:19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 1 and SEQ ID NO:2, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 1 and SEQ ID NO:2, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:3 and SEQ ID NO:4, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:5 and SEQ ID NO:6, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:7 and SEQ ID NO:8, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:9 and SEQ ID NO: 10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:9 and SEQ ID NO: 10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 11 and SEQ ID NO: 12; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having amino acid sequences set forth in SEQ ID NO:11 and SEQ ID NO: 12; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 13 and SEQ ID NO: 14, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 13 and SEQ ID NO: 14, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 15 and SEQ ID NO: 16, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 15 and SEQ ID NO: 16, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 19 and SEQ ID NO:20, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO: 19 and SEQ ID NO:20, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:21 and SEQ ID NO:22, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:21 and SEQ ID NO:22, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a FOLR1 antibody or antigen binding portion thereof or other binding agent comprising a heavy chain variable (VH) region and a light chain variable (VL) region, the VH and VL regions having amino acid sequences set forth in SEQ ID NO:23 and SEQ ID NO:24, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In any of these embodiments, the functional activity of the FOLR1 binding antibody or antigen binding portion thereof or other binding agent includes specifically binding to FOLR1. Additional functional activities include depletion of FOLR1+ cells (e.g., cancer cells). Additionally, a FOLR1 antibody or antigen binding portion thereof or other binding agent having functional activity means the polypeptide exhibits activity similar to, or better than, the activity of a reference antibody or antigen-binding portion thereof as described herein (e.g., a reference FOLR1 binding antibody or antigen binding portion thereof comprising (i) a heavy chain variable region having the amino acid sequence set forth in SEQ ID NO:36 and (ii) a light chain variable region having the amino acid sequence set forth in SEQ ID NO:37 or a variant thereof, as described herein), as measured in a particular assay, such as, for example, a biological assay, with or without dose dependency. In the case where dose dependency does exist, it need not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to Ile; Phe to Met, to Leu or to Tyr; Ser to Thr; Thr to Ser; Trp to Tyr; Tyr to Trp; and/or Phe to Val, to Ile or to Leu.

In some embodiments, a conservatively modified variant of a FOLR1 antibody or antigen binding portion thereof preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs are not modified. The degree of homology (percent identity) between the reference and modified sequence can be determined, for example, by comparing the two sequences using freely available computer programs commonly employed for this purpose on the world wide web (e.g. BLASTp or BLASTn with default settings).

In some embodiments, the VH and VL amino acid sequences collectively have no more than 8 or 6 or 4 or 2 or 1 conservative amino acid substitutions in the framework regions, as compared to the amino acid sequences of the unmodified VH and VL regions. In some embodiments, the VH and VL amino acid sequences collectively have 8 to 1, or 6 to 1, or 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions in the framework regions, as compared to the amino acid sequences of the unmodified VH and VL regions. In some embodiments, the VH and VL amino acid sequences have 8 to 1, 6 to 1, 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions, as compared to the amino acid sequences of the unmodified VH and VL regions.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

### Constant Regions

In some embodiments, a FOLR1 antibody or antigen-binding portion thereof or other binding agent has fully human constant regions. In some embodiments, a FOLR1 antibody or antigen-binding portion thereof or other binding agent has fully humanized constant regions. In some embodiments, a FOLR1 antibody or antigen-binding portion thereof or other binding agent has non-human constant regions. An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgGI, IgG2, IgG3, IgG4, or IgA1, and IgA2. The heavy-chain constant regions (Fc) that correspond to the different classes of immunoglobulins can be a, 0, E, y, and u, respectively. The light chains can be one of either kappa (or K) and lambda (or A).

In some embodiments, a constant region can have an IgGI isotype. In some embodiments, a constant region can have an IgG2 isotype. In some embodiments, a constant region can have an IgG3 isotype. In some embodiments, a constant region can have an IgG4 isotype. In some embodiments, an Fc domain can have a hybrid isotype comprising constant regions from two or more isotypes. In some embodiments, an immunoglobulin constant region can be an IgG1 or IgG4 constant region. In some embodiments, a FOLR1 antibody heavy chain is of the IgG1 isotype and has the amino acid sequence set forth in SEQ ID NO:39. In some embodiments, a FOLR1 antibody light chain is of the kappa isotype and has the amino acid sequence set forth in SEQ ID NO:40.

Furthermore, a FOLR1 antibody or an antigen-binding portion thereof or other binding agent may be part of a larger binding agent formed by covalent or noncovalent association of the antibody or antigen binding portion with one or more other proteins or peptides. Relevant to such binding agents are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (hexahistidinyl tag′ disclosed as SEQ ID NO: 41) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of a FOLR1 antibody or antigen binding portion thereof or other binding agent has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (e.g., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by of a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96TM non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202: 163 (1996); Cragg, M. S. et al., Blood 101: 1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A FOLR1 antibody or antigen binding portion thereof or other binding agent with reduced effector function activity includes those with substitution of one or more of Fc region residues, such as for example, 238, 265, 269, 270, 297, 327 and 329, according to the EU numbering of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU number of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A FOLR1 antibody or antigen binding portion thereof or other binding agent with diminished binding to FcRs can be prepared containing such amino acid modifications.

In some embodiments, a FOLR1 antibody or antigen binding portion thereof or other binding agent comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU number of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG) in an Fc region derived from a human IgG1 Fc region, according to the EU numbering of Kabat. (See, e.g., WO 2012/130831). In some embodiments, the substitutions are L234A, L235A and D265A (LALA-DA) in an Fc region derived from a human IgG1 Fc region, according to the EU number of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies, Antigen Binding Portions and Other Binding Agents

In various embodiments, FOLR1 antibodies, antigen binding portions thereof and other binding agents can be produced in human, murine or other animal-derived cells lines. Recombinant DNA expression can be used to produce FOLR1 antibodies, antigen binding portions thereof and other binding agents. This allows the production of FOLR1 antibodies as well as a spectrum of FOLR1 antigen binding portions and other binding agents (including fusion proteins) in a host species of choice. The production of FOLR1 antibodies, antigen binding portions thereof and other binding agents in bacteria, yeast, transgenic animals and chicken eggs are also alternatives for cell-based production systems. The main advantages of transgenic animals are potential high yields from renewable sources.

In some embodiments, a nucleic acid encodes a FOLR1 VH polypeptide having the amino acid sequence set forth in SEQ ID NOs: 1, 3, 5, 7, 9, 11, 13, 15, 17, 19, 21, or 23. In some embodiments, a nucleic acid encodes a FOLR1 VL polypeptide having the amino acid sequence set forth in SEQ ID NOs: 2, 4, 6, 8, 10, 12, 14, 16, 18, 20 or 22. In some embodiments, a nucleic acid encodes a FOLR1 VH polypeptide having the amino acid sequence set forth in SEQ ID NO:1. In some embodiments, a nucleic acid encodes a FOLR1 VH polypeptide having the amino acid sequence set forth in SEQ ID NO:3. In some embodiments, a nucleic acid encodes a FOLR1 VH polypeptide having the amino acid sequence set forth in SEQ ID NO:5. In some embodiments, a nucleic acid encodes a FOLR1 VH polypeptide having the amino acid sequence set forth in SEQ ID NO:7. In some embodiments, a nucleic acid encodes a FOLR1 VH polypeptide having the amino acid sequence set forth in SEQ ID NO:9. In some embodiments, a nucleic acid encodes a FOLR1 VH polypeptide having the amino acid sequence set forth in SEQ ID NO:11. In some embodiments, a nucleic acid encodes a FOLR1 VH polypeptide having the amino acid sequence set forth in SEQ ID NO:13. In some embodiments, a nucleic acid encodes a FOLR1 VH polypeptide having the amino acid sequence set forth in SEQ ID NO: 15. In some embodiments, a nucleic acid encodes a FOLR1 VH polypeptide having the amino acid sequence set forth in SEQ ID NO: 17. In some embodiments, a nucleic acid encodes a FOLR1 VH polypeptide having the amino acid sequence set forth in SEQ ID NO: 19. In some embodiments, a nucleic acid encodes a FOLR1 VH polypeptide having the amino acid sequence set forth in SEQ ID NO:21. In some embodiments, a nucleic acid encodes a FOLR1 VH polypeptide having the amino acid sequence set forth in SEQ ID NO:23.

In some embodiments, a nucleic acid encodes a FOLR1 VL polypeptide having the amino acid sequence set forth in SEQ ID NO:2. In some embodiments, a nucleic acid encodes a FOLR1 VL polypeptide having the amino acid sequence set forth in SEQ ID NO:4. In some embodiments, a nucleic acid encodes a FOLR1 VL polypeptide having the amino acid sequence set forth in SEQ ID NO:6. In some embodiments, a nucleic acid encodes a FOLR1 VL polypeptide having the amino acid sequence set forth in SEQ ID NO:8. In some embodiments, a nucleic acid encodes a FOLR1 VL polypeptide having the amino acid sequence set forth in SEQ ID NO: 10. In some embodiments, a nucleic acid encodes a FOLR1 VL polypeptide having the amino acid sequence set forth in SEQ ID NO: 12. In some embodiments, a nucleic acid encodes a FOLR1 VL polypeptide having the amino acid sequence set forth in SEQ ID NO: 14. In some embodiments, a nucleic acid encodes a FOLR1 VL polypeptide having the amino acid sequence set forth in SEQ ID NO:16. In some embodiments, a nucleic acid encodes a FOLR1 VL polypeptide having the amino acid sequence set forth in SEQ ID NO:18. In some embodiments, a nucleic acid encodes a FOLR1 VL polypeptide having the amino acid sequence set forth in SEQ ID NO:20. In some embodiments, a nucleic acid encodes a FOLR1 VL polypeptide having the amino acid sequence set forth in SEQ ID NO:22. In some embodiments, a nucleic acid encodes a FOLR1 VL polypeptide having the amino acid sequence set forth in SEQ ID NO:24.

In some embodiments, a nucleic acid encodes VH and VL polypeptides having the amino acid sequences set forth in SEQ ID NOs:1 and 2. In some embodiments, a nucleic acid encodes VH and VL polypeptides having the amino acid sequences set forth in SEQ ID NOs:3 and 4. In some embodiments, a nucleic acid encodes VH and VL polypeptides having the amino acid sequences set forth in SEQ ID NOs:5 and 6. In some embodiments, a nucleic acid encodes VH and VL polypeptides having the amino acid sequences set forth in SEQ ID NOs:7 and 8. In some embodiments, a nucleic acid encodes VH and VL polypeptides having the amino acid sequences set forth in SEQ ID NOs:9 and 10. In some embodiments, a nucleic acid encodes VH and VL polypeptides having the amino acid sequences set forth in SEQ ID NOs: 11 and 12. In some embodiments, a nucleic acid encodes VH and VL polypeptides having the amino acid sequences set forth in SEQ ID NOs: 13 and 14. In some embodiments, a nucleic acid encodes VH and VL polypeptides having the amino acid sequences set forth in SEQ ID NOs: 15 and 16. In some embodiments, a nucleic acid encodes VH and VL polypeptides having the amino acid sequences set forth in SEQ ID NOs: 17 and 18. In some embodiments, a nucleic acid encodes VH and VL polypeptides having the amino acid sequences set forth in SEQ ID NOs: 19 and 20. In some embodiments, a nucleic acid encodes VH and VL polypeptides having the amino acid sequences set forth in SEQ ID NOs:21 and 22 In some embodiments, a nucleic acid encodes VH and VL polypeptides having the amino acid sequences set forth in SEQ ID NOs:23 and 24.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding the amino acid sequence of a FOLR1 antibody, antigen binding portion thereof as well as other binding agents can be prepared by a variety of methods known in the art. These methods include, but are not limited to, preparation of synthetic nucleotide sequences encoding of a FOLR1 antibody, antigen binding portion or other binding agent(s). In addition, oligonucleotide-mediated (or site-directed) mutagenesis, PCR-mediated mutagenesis, and cassette mutagenesis can be used to prepare nucleotide sequences encoding a FOLR1 antibody or antigen binding portion as well as other binding agents. A nucleic acid sequence encoding at least a FOLR1 antibody, antigen binding portion thereof, binding agent, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases, or other techniques known in the art. Techniques for such manipulations are disclosed, e.g., by Maniatis et al., Molecular Cloning, Lab. Manual (Cold Spring Harbor Lab. Press, N Y, 1982 and 1989), and Ausubel et al., Current Protocols in Molecular Biology (John Wiley & Sons), 1987-1993, and can be used to construct nucleic acid sequences and vectors that encode a FOLR1 antibody or antigen binding portion thereof or a VH or VL polypeptide thereof or other binding agent.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., a FOLR1 antibody or antigen binding portion thereof or other binding agent) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a FOLR1 antibody or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a FOLR1 antibody or antigen binding portion thereof or other binding agent as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant FOLR1 antibodies or antigen-binding portions thereof or other binding agents. Known glycolytic genes can also provide very efficient transcriptional control signals. For example, the promoter and terminator signals of the phosphoglycerate kinase gene can be utilized.

Production of FOLR1 antibodies or antigen-binding portions thereof or other binding agents in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence (encoding a FOLR1 antibody or antigen binding portion thereof or other binding agent or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding FOLR1 antibodies or antigen-binding portions thereof or other binding agents include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the FOLR1 variable region(s) or antigen binding portions thereof or other binding agents are then transfected singly with nucleotides encoding a FOLR1 antibody or an antibody polypeptide or antigen-binding portion thereof or other binding agent, or are co-transfected with a polynucleotide(s) encoding VH and VL chain coding regions or other binding agents. The transfected recipient cells are cultured under conditions that permit expression of the incorporated coding regions and the expressed antibody chains or intact antibodies or antigen binding portions or other binding agents are recovered from the culture.

In some embodiments, the nucleic acids containing the coding regions encoding a FOLR1 antibody or antigen-binding portion thereof or other binding agent are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing FOLR1 antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the FOLR1 antibodies or antigen binding portions thereof or other binding agents, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding a FOLR1 antibody or antigen-binding portion thereof or other binding agent can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled FOLR1 antibodies and antigen binding portions thereof and other binding agents. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof and other binding agents as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of FOLR1 antibodies and antigen binding portions thereof and other binding agents in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof or other binding agents.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more FOLR1 antibodies or antigen-binding portions thereof or other binding agents can be produced in vivo in an animal that has been engineered or transfected with one or more nucleic acid molecules encoding the polypeptides, according to any suitable method.

In some embodiments, an antibody or antigen-binding portion thereof or other binding agent is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof or other binding agents can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains in conjunction with additional selectable markers to generate cell lines with enhanced properties, such as higher production of assembled FOLR1 antibodies or antigen binding portions thereof or other binding agents or enhanced stability of the transfected cell lines.

Additionally, plants have emerged as a convenient, safe and economical alternative expression system for recombinant antibody production, which are based on large scale culture of microbes or animal cells. FOLR1 binding antibodies or antigen binding portions thereof or other binding agents can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of the FOLR1 antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). A FOLR1 binding antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989);

which are incorporated by reference herein in their entireties) can be adapted to produce single chain antibodies that specifically bind to FOLR1. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion or other binding agent comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). ScFv-Fcs have been described by Sokolowska-Wedzina et al., Mol. Cancer Res. 15(8):1040-1050, 2017.

In some embodiments, an antigen binding portion or other binding agent is a single-domain antibody which is an antigen binding portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259 and Li et al., Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324: 13-25, 2007.) A VHH may have potent antigen-binding capacity and can interact with epitopes that are inacessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5): 1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be binding agents (see, e.g. US 2006/0025576A1).

The binding agents (e.g., antibodies or antigen binding portions) herein also include a “Dual Acting FAb” or “DAF” comprising an antigen binding site that binds to two different antigens (see, e.g., US 2008/0069820 and Bostrom et al., 2009, Science 323:1610-14). “Crossmab” antibodies are also included herein (see e.g. WO 2009/080251, WO 2009/080252, WO2009/080253, WO2009/080254, and WO2013/026833).

In some embodiments, the binding agents comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the binding agent a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a binding agent is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a binding agent that is a bispecific antibody is composed of a single polypeptide chain comprising two single chain FV portions (scFV) fused to each other by a peptide linker.

In some embodiments, a binding agent is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2): 182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7): 1803-1812, 2014; and Bates and Power, Antibodies, 2019, 8, 28.

Igg-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7): 838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof and other binding agents can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure FOLR1 binding antibodies or antigen binding portions thereof or other binding agents of at least about 90% to 95% homogeneity are advantageous, as are those with 98% to 99% or more homogeneity, particularly for pharmaceutical uses. Once purified, partially or to homogeneity as desired, an intact FOLR1 antibody or antigen binding portions thereof or other binding agent can then be used therapeutically or in developing and performing assay procedures, immunofluorescent staining, and the like. See generally, Vols. I & II Immunol. Meth. (Lefkovits & Pernis, eds., Acad. Press, N Y, 1979 and 1981).

### Antibody Drug Conjugates

In some embodiments, a FOLR1 antibody, antigen binding portion or other binding agent as described herein is part of a FOLR1 antibody drug conjugate (also referred to as a FOLR1 conjugate or FOLR1 ADC). In some embodiments, the FOLR1 antibody, antigen binding portion or other binding agent is attached to at least one linker, and at least one drug is attached to each linker. As used herein, in the context of a conjugate, the term “drug” refers to cytotoxic agents (such as chemotherapeutic agents or drugs), immunomodulatory agents, nucleic acid (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, a FOLR1 conjugate includes at least one drug that is cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, or ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide AI-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a drug is an immune modulatory agent. An immune modulatory agent can be, for example, a TLR7 and/or TLR8 agonist, a STING agonist, or RIG-I agonist or other immune modulatory agent.

In some embodiments, a drug is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences).

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics), WO2018198091 (Novartis AG), and US20170131421 (Novartis AG).

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-I agonist. Examples of RIG-I agonists include KIN1148, SB-9200, KIN700, KIN600, KIN500, KIN100. KIN101, KIN400 and KIN2000.

### Toxins

] In some embodiments, a drug is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a drug is a radioactive atom. A variety of radioactive isotopes are available for the production of radioconjugates. Examples include I131, I125, Y90, Re186, Re188, Sm153, Bi213, P32, Pb212 and radioactive isotopes of Lutetium (e.g., Lu177.

### PROTACs

In some embodiments, a drug is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Linkers

The FOLR1 conjugates typically comprise at least one linker, each linker having at least one drug attached to it. Typically, a conjugate includes a linker between a FOLR1 antibody (or antigen binding portion thereof or other binding agent) and the drug. In various embodiments, a linker may be a protease cleavable linker, an acid-cleavable linker, a disulfide linker, a disulfide-containing linker or a disulfide containing linker having a dimethyl group adjacent the sulfide bond (see, e.g., Jain et al., Pharm. Res. 32:3526-3540 (2015); Chari et al., Cancer Res. 52:127-131 (1992); U.S. Pat. No. 5,208,020), a self-stabilizing linker (see, e.g., WO2018/031690; WO2015/095755 and Jain et al., Pharm. Res. 32:3526-3540 (2015)), a non-cleavable linker (see, e.g., WO2007/008603), a photolabile linker, and/or a hydrophilic linker (see, e.g., WO2015/123679).

In some embodiments, a linker is a cleavable linker that is cleavable under intracellular conditions, such that cleavage of the linker releases the drug from the antibody (or antigen binding portion thereof or other binding agent) and/or linker in the intracellular environment. For example, in some embodiments, a linker is cleavable by a cleaving agent that is present in the intracellular environment (e.g., within a lysosome or endosome or caveolae). A linker can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Typically, a peptidyl linker is at least one amino acid long or at least two amino acids long. Intracellular cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO: 42). Other such linkers are described, e.g., in U.S. Pat. No. 6,214,345. In specific embodiments, a peptidyl linker cleavable by an intracellular protease is a Val-Cit linker or a Phe-Lys linker (see, e.g., U.S. Pat. No. 6,214,345, which describes the synthesis of doxorubicin with the val-cit linker) or Gly-Gly-Phe-Gly linker (SEQ ID NO: 43) (see, e.g., US2015/0297748). One advantage of using intracellular proteolytic release of the drug is that the drug is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between a drug (e.g., a cytotoxic agent) and the antibody is broken, resulting in the free drug, or other metabolite of the conjugate dissociated from the antibody inside the cell. The cleaved moieties of the conjugate are thus intracellular metabolites.

In some embodiments, a cleavable linker is pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, a hydrolyzable linker is a thioether linker (such as, e.g., a thioether attached to a drug via an acylhydrazone bond (see, e.g., U.S. Pat. No. 5,622,929)).

In some embodiments, a linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, the linker is a malonate linker (Johnson et al., 1995, Anticancer Res. 15:1387-93), a maleimidobenzoyl linker (Lau et al., 1995, Bioorg-Med-Chem. 3(10): 1299-1304), or a 3′-N-amide analog (Lau et al., 1995, Bioorg-Med-Chem. 3(10): 1305-12). In some embodiments, the linker unit is not cleavable, such as a maleimidocaproyl linker, and the drug is released by antibody degradation. (See U.S. Publication No. 2005/0238649).

In some embodiments, a linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC) are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or drug (the “control sample”) for a predetermined time period (e.g., 2, 4, 8, 16, or 24 hours) and then comparing the amount of unconjugated antibody or drug present in the ADC sample with that present in control sample, as measured, for example, by high performance liquid chromatography.

In some embodiments, a linker promotes cellular internalization. In some embodiments, a linker promotes cellular internalization when conjugated to the drug such as a cytotoxic agent (i.e., in the milieu of the linker-drug of the ADC as described herein). In yet other embodiments, a linker promotes cellular internalization when conjugated to both the drug and the FOLR1 antibody (i.e., in the milieu of the ADC as described herein).

A variety of linkers that can be used with the present compositions and methods are described in WO 2004010957. In some embodiments, a protease cleavable linker comprises a thiol-reactive spacer and a dipeptide. In some embodiments, the protease cleavable linker consists of a thiol-reactive maleimidocaproyl spacer, a valine-citrulline dipeptide, and a p-amino-benzyloxycarbonyl spacer.

In some embodiments, an acid cleavable linker is a hydrazine linker or a quaternary ammonium linker (see WO2017/096311 and WO2016/040684.)

In some embodiments, a linker is a self-stabilizing linker comprising a maleimide group as described in U.S. Pat. No. 9,504,756.

In some embodiments, a linker is a hydrophilic linker, such as, for example, the hydrophilic peptides in WO2015/123679 and the sugar alcohol polymer-based linkers disclosed in WO2013/012961 and WO2019/213046.

In other embodiments, conjugates of a FOLR1 antibody (or antigen binding portion or other binding agent) and a drug may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) to an antibody, antigen binding portion thereof or other binding agent have been described in, for example WO94/11026.

The conjugates of a FOLR1 antibodies (or antigen binding portion or other binding agent) include, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In some embodiments, a linker is attached to a terminus of an amino acid sequence of an antibody, antigen binding portion or other binding agent or can be attached to a side chain modification of an antibody, antigen binding portion or other binding agent, such as the side chain of a lysine, serine, threonine, cysteine, tyrosine, aspartic acid, a non-natural amino acid residue, glutamine, or glutamic acid residue. An attachment between an antibody, antigen binding portion or other binding agent and a linker or drug can be via any of a number of bonds, for example but not limited to, an amide bond, an ester bond, an ether bond, a carbon-nitrogen bond, a carbon-carbon single double or triple bond, a disulfide bond, or a thioether bond. Functional groups that can form such bonds include, for example, amino groups, carboxyl groups, aldehyde groups, azide groups, alkyne and alkene groups, ketones, carbonates, carbonyl functionalities bonded to leaving groups such as cyano and succinimidyl and hydroxyl groups.

In some embodiments, a linker is attached to an antibody, antigen binding portion or other binding agent at an interchain disulfide. In some embodiments, a linker is connected to an antibody, antigen binding portion or other binding agent at a hinge cysteine residue. In some embodiments, a linker is attached to an antibody, antigen binding portion or other binding agent at an engineered cysteine residue. In some embodiments, a linker is connected to an antibody, antigen binding portion or other binding agent at a lysine residue. In some embodiments, a linker is connected to an antibody, antigen binding portion or other binding agent at an engineered glutamine residue. In some embodiments, a linker is connected to an antibody, antigen binding portion or other binding agent at an unnatural amino acid engineered into the heavy chain.

In some embodiments, a linker is attached to an antibody, antigen binding portion or other binding agent via a sulfhydryl group. In some embodiments, a linker is attached to an antibody, antigen binding portion or other binding agent via a primary amine. In some embodiments, a linker is attached via a link created between an unnatural amino acid on an antibody, antigen binding portion or other binding agent by reacting with oxime bond that was formed by modifying a ketone group with an alkoxyamine on a drug.

In some embodiments, a linker is attached to an antibody, antigen binding portion or other binding agent via Sortase A linker. A Sortase A linker can be created by a Sortase A enzyme fusing an LPXTG recognition motif (SEQ ID NO: 44) to an N-terminal GGG motif to regenerate a native amide bond.

### Exemplary Linker Drug Combinations

In some embodiments, a drug such as a tubulin disrupting agent, for example, an auristatin, is attached to a linker by a C-terminal carboxyl group that forms an amide bond with a linker (e.g., a Linker Unit (LU)) as described in U.S. Pat. No. 9,463,252, incorporated herein by reference). In some embodiments, a linker comprises at least one amino acid.

In some embodiments, a linker also comprises a stretcher unit and/or an amino acid unit. Exemplary stretcher units and amino acid units are described in U.S. Pat. Nos. 9,345,785 and 9,078,931, each of which is herein incorporated by reference.

In some embodiments, an antibody drug conjugate comprises an anti-FOLR1 antibody covalently linked to MMAE through an mc-val-cit-PAB linker.

In some embodiments, the FOLR1 conjugates have the following formula:

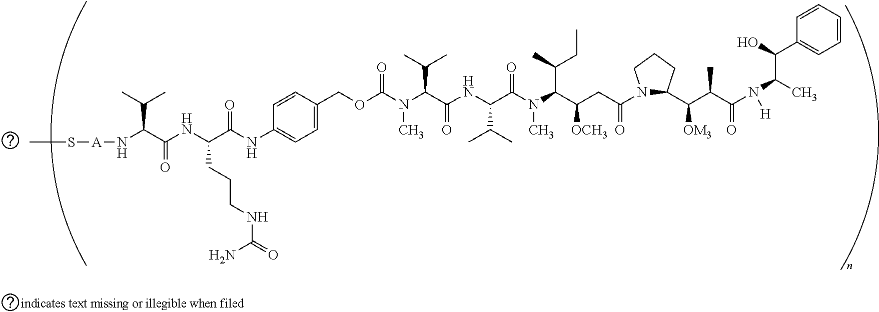

or a pharmaceutically acceptable salt thereof; wherein: mAb is a FOLR1 antibody, antigen binding portion thereof or other binding agent, S is a sulfur atom of the antibody, antigen binding portion or other binding agent, A is a Stretcher unit, and p is from about 3 to about 5, or from about 3 to about 8.

The drug loading is represented by p, the average number of drug molecules (e.g., cytotoxic agents) per antibody (or antigen binding portion or other binding agent) in a conjugate. For example, if p is about 4, the average drug loading taking into account all of the antibody (or antigen binding portion or other binding agent) present in the composition is about 4. In some embodiments, p ranges from about 3 to about 5, from about 3.6 to about 4.4, or from about 3.8 to about 4.2. In some embodiments, p can be about 3, about 4, or about 5. In some embodiments, p ranges from about 6 to about 8, more preferably from about 7.5 to about 8.4. In some embodiments, p can be about 6, about 7, or about 8.

The average number of drugs per antibody (or antigen binding portion or other binding agent) in a preparation may be characterized by conventional means such as mass spectroscopy, ELISA assay, and HPLC. The quantitative distribution of antibody-drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

In some embodiments, a stretcher unit is capable of linking an antibody (or antigen binding portion or other binding agent) to an amino acid or peptide (e.g., a valine-citrulline peptide) via a sulfhydryl group of the antibody (or antigen binding portion or other binding agent). Sulfhydryl groups can be generated, for example, by reduction of the interchain disulfide bonds of a FOLR1 antibody (or antigen binding portion or other binding agent). For example, a stretcher unit can be linked to the antibody (or antigen binding portion or other binding agent) via the sulfur atoms generated from reduction of the interchain disulfide bonds of an antibody (or antigen binding portion or other binding agent). In some embodiments, stretcher units are linked to the antibody (or antigen binding portion or other binding agent) solely via the sulfur atoms generated from reduction of the interchain disulfide bonds of the antibody. In some embodiments, sulfhydryl groups can be generated by reaction of an amino group of a lysine moiety of a FOLR1 antibody (or antigen binding portion or other binding agent) with 2-iminothiolane (Traut's reagent) or other sulfhydryl generating reagents. In some embodiments, a FOLR1 antibody (or antigen binding portion or other binding agent) is a recombinant antibody and is engineered to carry one or more lysines. In some embodiments, a recombinant FOLR1 antibody (or antigen binding portion or other binding agent) is engineered to carry additional sulfhydryl groups, e.g., additional cysteines, such as engineered cysteines.

The synthesis and structure of MMAE is described in U.S. Pat. No. 6,884,869 incorporated by reference herein in its entirety and for all purposes. The synthesis and structure of exemplary stretcher units and methods for making antibody drug conjugates are described in, for example, U.S. Publication Nos. 2006/0074008 and 2009/0010945, each of which is incorporated herein by reference in its entirety.

Representative stretcher units are described within the square brackets of Formulas IIIa and IIIb of U.S. Pat. No. 9,211,319, and incorporated herein by reference.

In some embodiments, a FOLR1 conjugate comprises monomethyl auristatin E (MMAE) and a protease-cleavable linker. It is contemplated that the protease cleavable linker comprises a thiol-reactive spacer and a dipeptide. In various embodiments, the protease cleavable linker includes a thiol-reactive maleimidocaproyl spacer, a valine-citrulline (val-cit) dipeptide, and a p-amino-benzyloxycarbonyl or PAB spacer.

The abbreviation “PAB” refers to the self-immolative spacer:

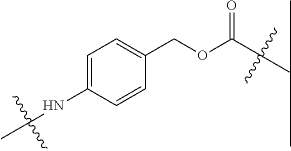 [SMILES: *Nc1ccc(COC(*)=O)cc1]

The abbreviation “MC” refers to the stretcher maleimidocaproyl:

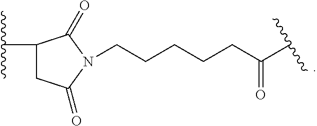 [SMILES: *C(=O)CCCCCN1C(=O)CC(*)C1=O]

In other exemplary embodiments, a conjugate has the following general formula:

Ab-[L3]-[L2]-[L1]m-AAn-drug,

where Ab is a FOLR1 antibody (or antigen binding portion or other binding agent); the drug can be, for example, a cytotoxic agent such as a tubulin-disrupting agent or topoisomerase inhibitor; L3 is a component of a linker comprising an antibody-coupling moiety (such as a stretcher unit) and one or more of acetylene (or azide) groups; L2 comprises an optional PEG (polyethylene glycol) azide (or acetylene) at one end, complementary to the acetylene (or azide) moiety in L3, and a reactive group such as carboxylic acid or hydroxyl group at the other end; L1 comprises a collapsible unit (e.g., a self-immolative group(s)), or a peptidase-cleavable moiety optionally attached to a collapsible unit, or an acid-cleavable moiety; AA is an amino acid; m is an integer with values of 0 or 1, and n is an integer with values of 0, 1, 2, 3, or 4. Such linkers can be assembled via click chemistry. (See, e.g., U.S. Pat. Nos. 7,591,944 and 7,999,083.)

In some embodiments, the drug is a camptothecin or a camptothecin (CPT) analog, such as irinotecan (also referred to as CPT-11), belotecan, topotecan, 10-hydroxy-CPT, exatecan, DXd and/or SN-38. Representative structures are shown below.

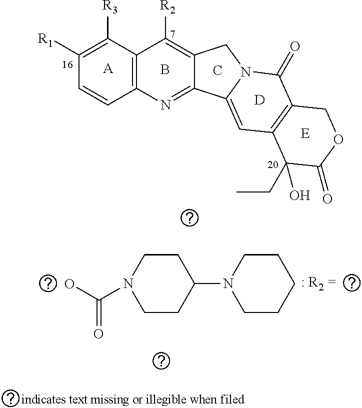

Referring to the conjugate formula Ab-[L3]-[L2]-[L1]m-AAn-drug, in some embodiments, m is 0. Referring to the conjugate formula Ab-[L3]-[L2]-[L1]m-AAn-drug, in some embodiments, L2 is absent. In such embodiments, an ester moiety is first formed between the carboxylic acid of an amino acid (AA) such as glycine, alanine, or sarcosine, or of a peptide such as glycylglycine, and a hydroxyl group of a drug, such as cytotoxic agent. In this example, the N-terminus of the amino acid or polypeptide may be protected as a Boc or a Fmoc or a monomethoxytrityl (MMT) derivative, which is deprotected after formation of an ester bond with the hydroxyl group of the cytotoxic agent. Selective removal of an amine-protecting group, in the presence of a BOC protecting group at a hydroxyl position of the cytotoxic agent containing an additional hydroxyl group(s) can be achieved using monomethoxytrityl (MMT) as the protecting group for the amino group of amino acid or polypeptide involved in ester formation, since ‘MMT’ is removable by mild acid treatment such as dichloroacetic acid that does not cleave a BOC group. After the amino group of the amino acid or polypeptide, forming an ester bond with hydroxyl of the drug, is demasked, the amino group is reacted with the activated form of a COOH group on PEG moiety of L2 (if present) under standard amide-forming conditions. In a preferred embodiment, L3 comprises a thiol-reactive group which links to a thiol group(s) of an antibody (or an antigen binding portion or other binding agent). The thiol-reactive group is optionally a maleimide or vinylsulfone, or bromoacetamide, or iodoacetamide, which links to a thiol group of the antibody. In some embodiments, the reagent bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In another embodiments, m is 0, and AA comprises a peptide moiety, preferably a di, tri or tetrapeptide, that is cleavable by intracellular peptidase such as Cathepsin-B. Examples of cathepsin-B-cleavable peptides are: Phe-Lys, Val-Cit (Dubowchick, 2002), Ala-Leu, Leu-Ala-Leu, Ala-Leu-Ala-Leu (SEQ ID NO: 45) (Trouet et al., 1982), and Gly-Gly-Phe-Gly (SEQ ID NO: 43) (see, e.g., WO2014/057687).

In some embodiments, L1 is composed of intracellularly-cleavable peptide, such as cathepsin-B-cleavable peptide, connected to the collapsible unit, such as p-aminobenzyl alcohol (or p-amino-benzyloxycarbonyl) at the peptide's C-terminus, the benzyl alcohol portion of which is in turn directly attached to a hydroxyl group of the drug, such as a cytotoxic agent, in chloroformate form. In this embodiment, n is 0. Alternatively, when ‘n’ is non-zero, the benzyl alcohol portion of the p-amidobenzyl alcohol (or p-amino-benzyloxycarbonyl) moiety is attached to the N-terminus of the amino acid or peptide linking at the hydroxyl group of the cytotoxic agent through the activated form of p-amidobenzyl alcohol, namely PABOCOPNP where PNP is p-nitrophenyl. In some embodiments, the linker comprises a thiol-reactive group which links to thiol groups of the antibody (or antigen binding portion or other binding agent). The thiol-reactive group is optionally a maleimide or vinylsulfone, or bromoacetamide, or iodoacetamide, which links to thiol groups of the antibody (or antigen binding portion or other binding agent). In a preferred embodiment, the component bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In some embodiments, where the drug is a cytotoxic agent is a camptothecin or analog or derivative thereof having a 20-hydroxyl, L1 is composed of intracellularly-cleavable peptide, such as cathepsin-B-cleavable peptide, connected to the collapsible linker p-aminobenzyl alcohol (or p-amino-benzyloxycarbonyl) at the peptide's C-terminus, the benzyl alcohol portion of which is in turn directly attached to CPT-20-O-chloroformate. In this embodiment, n is 0. Alternatively, when ‘n’ is non-zero, the benzyl alcohol portion of the p-amidobenzyl alcohol moiety is attached to the N-terminus of the amino acid or polypeptide linking at CPT's 20 position through the activated form of p-amidobenzyl alcohol, namely PABOCOPNP where PNP is p-nitrophenyl. In a preferred embodiment, the linker comprises a thiol-reactive group which links to thiol groups of an antibody (or antigen binding portion or other binding agent). The thiol-reactive group is optionally a maleimide or vinylsulfone, or bromoacetamide, or iodoacetamide, which links to thiol groups of an antibody (or antigen binding portion or other binding agent). In a preferred embodiment, the component bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In some embodiments, the L2 component of the conjugate is present and contains a polyethylene glycol (PEG) spacer that can be of up to about MW 5000 in size, and in a preferred embodiment, PEG is a defined PEG with (1-12 or 1-30) repeating monomeric units. In some embodiments, PEG is a defined PEG with 1-12 repeating monomeric units. The introduction of PEG may involve using heterobifunctionalized PEG derivatives which are available commercially. The heterobifunctional PEG typically contains an azide or acetylene group. An example of a heterobifunctional defined PEG containing 8 repeating monomeric units, with ‘NHS’ being succinimidyl, is given below in the following formula:

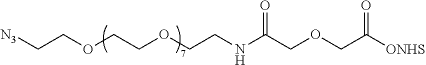

In some embodiments, L3 has a plurality of acetylene (or azide) groups, ranging from 2-40, but preferably 2-20, and more preferably 2-5, and a single antibody binding moiety.

A representative conjugate, in which the drug is a cytotoxic agent such as SN-38 (a CPT analog), prepared with a maleimide-containing SN-38-linker derivative, with the bonding to an antibody (designated MAb) represented as a succinimide, is given below. Here, m=0, and the 20-O-AA ester bonding to SN-38 is glycinate; azide-acetylene coupling joining of L2 and L3 results in the triazole moiety as shown.

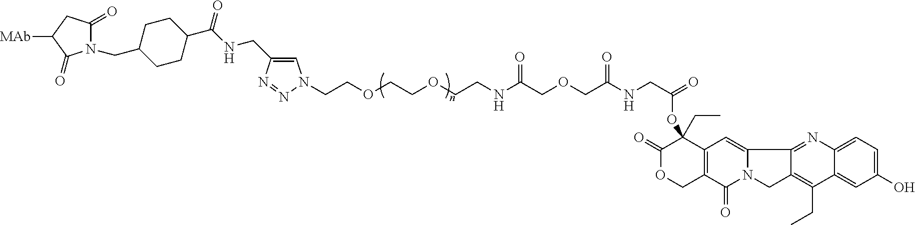

In another representative conjugate, prepared with a maleimide-containing SN-38-linker derivative, with the bonding to an antibody (MAb) represented as a succinimide, is shown below. Here, n=0 in the general formula 2; ‘L1’ contains a cathepsin-B-cleavable dipeptide, Phe-Lys, attached to the collapsible p-aminobenzyl alcohol moiety, and the latter is attached to SN-38 as a carbonate bonding at the 20 position; azide-acetylene coupling joining the ‘L2’ and ‘L3’ parts results in the triazole moiety as shown.

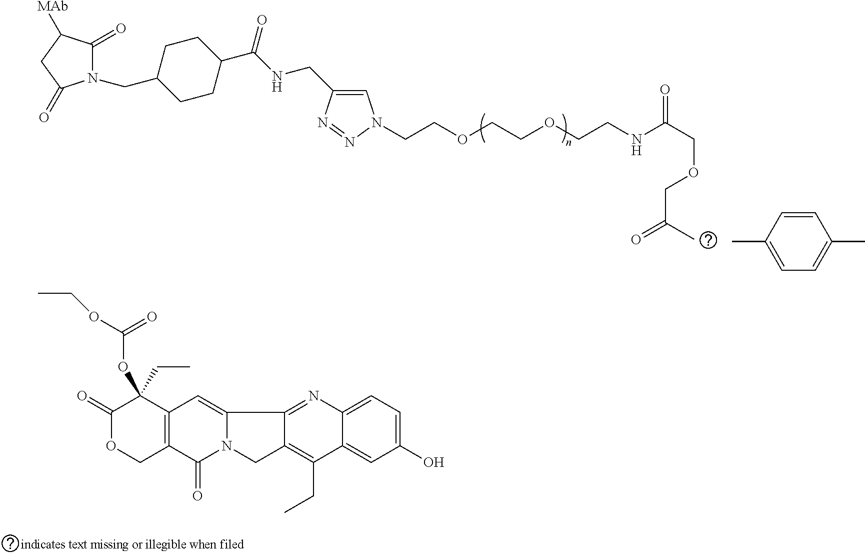

Another representative SN-38 conjugate, mAb-CL2-SN-38, is prepared with a maleimide-containing SN-38-linker derivative, with the bonding to an antibody represented as a succinimide, is given below. Here, the 20-O-AA ester bonding to SN-38 is glycinate that is attached to L1 portion via a p-aminobenzyl alcohol moiety and a cathepsin-B-cleavable dipeptide; the latter is in turn attached to ‘L2’ via an amide bond, while ‘L2’ and ‘L3’ parts are coupled via azide-acetylene ‘click chemistry’.

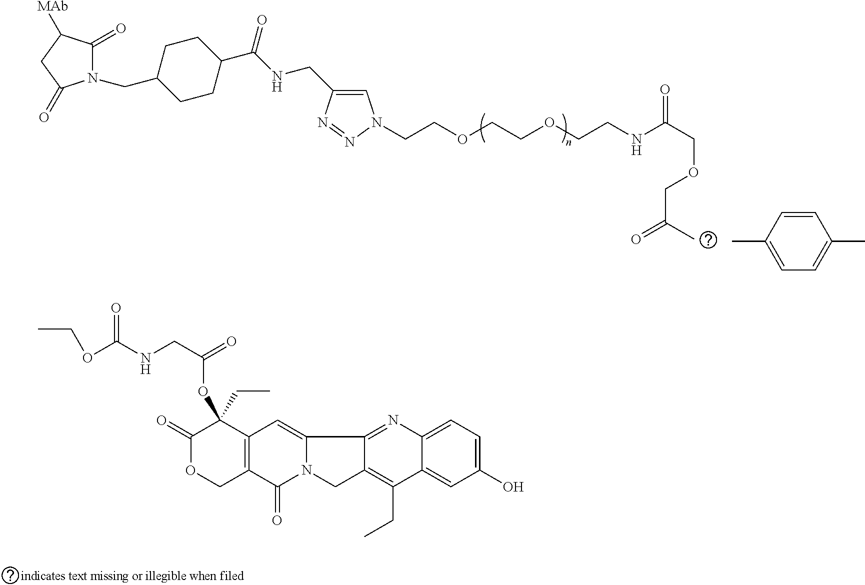

In another representative example, ‘L1’ contains a single amino acid attached to the collapsible p-aminobenzyl alcohol moiety, where the p-aminobenzyl alcohol is substituted or unsubstituted (R), where m=1 and n=0 in the general conjugate formula, Ab-[L3]-[L2]-[L1]m-AAn-drug, and the drug is exemplified with SN-38. The structure is represented below (referred to as MAb-CLX-SN-38). Single amino acid of AA can be selected from any one of the following L-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The substituent R on 4-aminobenzyl alcohol moiety is hydrogen or an alkyl group selected from C1-C10 alkyl groups.

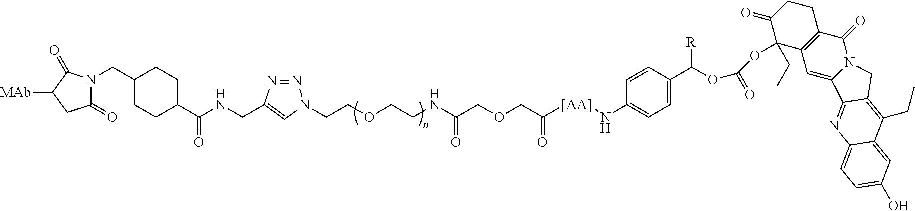

An embodiment of mAb-CLX-SN-38 (above), wherein the single amino acid AA is L-lysine and R═H, and the drug is a cytotoxic agent exemplified by SN-38 (referred to as mAb-CL2A-SN-38) is shown below:

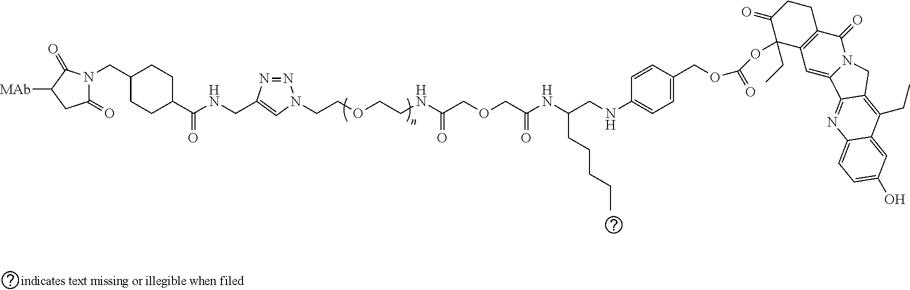

In other embodiments, a drug is a cytotoxic agent that is attached to a linker comprising a stretcher unit (Z) attached to an Amino Acid unit (AA) attached to a Spacer unit (Y), where the stretcher unit is attached to the antibody (or antigen binding portion thereof or other binding agent, designated Ab or MAb) and the Spacer unit is attached to an amino group of a cytotoxic agent. Such a linker has the following formula:

Ab-Z-AA-Y-cytotoxic agent,

where Z is selected from -(Succinimid-3-yl-N)—(CH₂)ₙ²—C(═O)—, —CH₂—C(═O)—NH—(CH₂)n³—C(═O)—, —C(═O)-cyc·Hex(1,4)-CH₂—(N-ly-3-diminiccuS)-, or —C(═O)—(CH₂)n⁴-C(═O)—, wherein n² represents an integer of 2 to 8, n³ represents an integer of 1 to 8, and n⁴ represents an integer of 1 to 8; cyc. Hex(1,4) represents a 1,4-cyclohexylene group; and (N-ly-3-diminiccuS)- has a structure represented by the following formula:

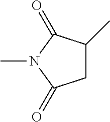

In some embodiments, AA is a peptide of from 2 to 7 amino acids. In some embodiments, the spacer unit Y is —NH—(CH₂)_{b}—(C═O)— or —NH—CH₂—O—CH₂—(C═O)—, where b is an integer from 1 to 5.

In some embodiments, the cytotoxic agent is exatecan. In some embodiments, the amino acid unit (AA) is -Gly-Gly-Phe-Gly-. In some embodiments, the spacer unit Y is —NH—CH₂—O—CH₂—(C═O)—.

In some embodiments, the linker-cytotoxic agent has the following structure:

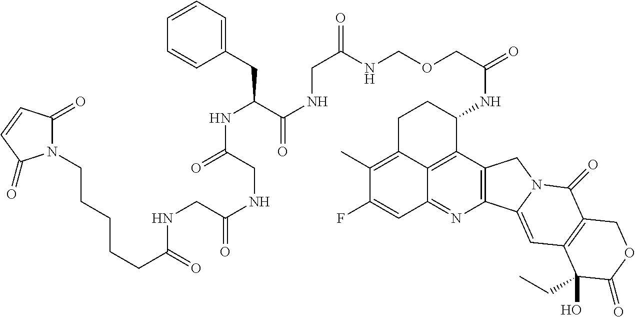 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

where the released cytotoxic agent is DXd (see U.S. Pat. No. 9,808,537).

### Attachment of Drug-Linkers to Antibodies, Antibody Binding Portions and Other Binding Agents

Techniques for attaching drugs to antibodies (or antigen binding portions thereof or other binding agents) via linkers are well-known in the art. See, e.g., Alley et al., Current Opinion in Chemical Biology 2010 14:1-9; Senter, Cancer J., 2008, 14(3): 154-169. In some embodiments, a linker is first attached to a drug (e.g., a cytotoxic agent(s)) and then the drug-linker is attached to the antibody or antigen binding portion thereof or other binding agent. In some embodiments, a linker is first attached to an antibody or antigen binding portion thereof or other binding agent, and then a drug is attached to the linker. In the following discussion, the term drug-linker is used to exemplify attachment of linkers or drug-linkers to antibodies or antigen binding portions thereof or other binding agents; the skilled artisan will appreciate that the selected attachment method can be selected according to linker and the cytotoxic agent or other drug. In some embodiments, a drug is attached to an antibody or antigen binding portion thereof or other binding agent via a linker in a manner that reduces the activity of the drug until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of an antibody (or antigen binding portion thereof or other binding agent) with a bivalent linker reagent to form an antibody-linker intermediate via a covalent bond, followed by reaction with a drug (e.g., a cytotoxic agent); and (2) reaction of a nucleophilic group of a drug (e.g., a cytotoxic agent) with a bivalent linker reagent, to form drug-linker, via a covalent bond, followed by reaction with a nucleophilic group of an antibody or antigen binding portion thereof or other binding agent. Exemplary methods for preparing conjugates via the latter route are described in U.S. Pat. No. 7,498,298, which is expressly incorporated herein by reference.

Nucleophilic groups on antibodies, antigen binding portions and other binding agents include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies (and antigen binding portions and other binding agents) have reducible interchain disulfides, i.e. cysteine bridges. Antibodies (and antigen binding portions and other binding agents) may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies (and antigen binding portions and other binding agents) through modification of lysine residues, e.g., by reacting lysine residues with 2-iminothiolane (Traut's reagent), resulting in conversion of an amine into a thiol. Reactive thiol groups may also be introduced into an antibody (and antigen binding portions and other binding agents) by introducing one, two, three, four, or more cysteine residues (e.g., by preparing antibodies, antigen binding portions and other binding agents comprising one or more non-native cysteine amino acid residues).

Conjugates may also be produced by reaction between an electrophilic group on an antibody (or antigen binding portion thereof or other binding agent), such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In an embodiment, an antibody (or antigen binding portion thereof or other binding agent) is modified to introduce electrophilic moieties that are capable of reacting with nucleophilic substituents on the linker reagent or drug. In another embodiment, the sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent) that can react with appropriate groups on the drug (see, e.g., Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a cytotoxic agent or linker.

Exemplary nucleophilic groups on a drug, such as a cytotoxic agent, include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linkers that may be used to prepare a conjugate are described herein or are known to persons of ordinary skill in the art. Methods of using such cross-linkers to link two moieties, including an antibody (or antigen binding portion or other binding agent) and a chemical moiety, are known in the art. In some embodiments, a fusion protein comprising an antibody or antigen binding portion and a drug may be made, e.g., by recombinant techniques or peptide synthesis. A recombinant DNA molecule may comprise regions encoding the antibody (or antigen binding portion thereof or other binding agent) and active portions (e.g., cytotoxic portions) of the conjugate either adjacent to one another or separated by a region encoding a linker which does not destroy the desired properties of the conjugate.

In some embodiments, a drug-linker is attached to an interchain cysteine residue(s) of an antibody (or antigen binding portion thereof or other binding agent). See, e.g., WO2004/010957 and WO2005/081711. In such embodiments, the linker typically comprises a maleimide group for attachment to the cysteine residues of an interchain disulfide. In some embodiments, the linker or drug-linker is attached to a cysteine residue(s) of an antibody or antigen binding portion thereof as described in U.S. Pat. Nos. 7,585,491 or 8,080,250. The drug loading of the resulting conjugate typically ranges from 1 to 8.

In some embodiments, the linker or drug-linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents.

In some embodiments, a drug-linker(s) is attached to an engineered cysteine residue at an Fc residue other than an interchain disulfide. In some embodiments, a drug-linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a linker or drug-linker(s) is attached to one or more introduced cysteine residues of an antibody (or antigen binding portion thereof or other binding agent) as described in WO2006/034488, WO2011/156328 and/or WO2016040856.

In some embodiments, an exemplary substitution for site specific conjugation using bacterial transglutaminase is N297S or N297Q of the Fc region. In some embodiments, a linker or drug-linker(s) is attached to the glycan or modified glycan of an antibody or antigen binding portion or a glycoengineered antibody (or other binding agent). See, e.g., WO2017/147542, WO2020123425, WO2014/072482; WO2014//065661, WO2015/057066 and WO2016/022027.

### Pharmaceutical Formulations

Other aspects of the FOLR1 antibodies and antigen binding portions thereof or other binding agents and conjugates of any of these relate to compositions comprising active ingredients (i.e., including a FOLR1 antibody or antigen-binding portion thereof or other binding agent or conjugate thereof as described herein or a nucleic acid encoding an antibody or antigen-binding portion thereof or other binding agent as described herein). In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A FOLR1 antibody or antigen binding portion thereof or other binding agent or conjugate thereof can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a FOLR1 antibody or antigen binding portion thereof or other binding agent or conjugate thereof). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., a FOLR1 antibody and/or antigen binding portions thereof other binding agent or conjugate thereof) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a FOLR1 antibody or antigen-binding portion thereof or other binding agent or conjugate thereof as described herein or a nucleic acid encoding a FOLR1 antibody or antigen-binding portion thereof or other binding agent as described herein can be a lyophilisate.

In some embodiments, a syringe comprising a therapeutically effective amount of a FOLR1 antibody or antigen binding portion thereof or other binding agent or conjugate thereof, or a pharmaceutical composition described herein is provided.

### Treatment of Cancer

In some embodiments, the FOLR1 antibodies or antigen binding portions thereof, binding agents and conjugates as described herein can be used in a method(s) comprising administering a FOLR1 antibody or antigen-binding portion thereof or other binding agent or conjugate thereof as described herein to a subject in need thereof, such as to a subject having cancer.

In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in the pairs of amino acid sequences selected from SEQ ID NO: 1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; SEQ ID NO: 11 and SEQ ID NO: 12; respectively; SEQ ID NO:13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO: 1 and SEQ ID NO:2, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO:3 and SEQ ID NO:4, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO:5 and SEQ ID NO:6, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO:7 and SEQ ID NO:8, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO:9 and SEQ ID NO: 10, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in and SEQ ID NO: 11 and SEQ ID NO: 12; respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in and SEQ ID NO: 13 and SEQ ID NO: 14; respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO: 15 and SEQ ID NO: 16, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO: 17 and SEQ ID NO: 18, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO: 19 and SEQ ID NO:20, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO:21 and SEQ ID NO:22, respectively. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in SEQ ID NO:23 and SEQ ID NO:24, respectively.

In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO:10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, provided are methods comprising administering a FOLR1 antibody or antigen-binding portion thereof or other binding agent or conjugate thereof comprising a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:3 and SEQ ID NO:4, respectively; SEQ ID NO:5 and SEQ ID NO:6, respectively; SEQ ID NO:7 and SEQ ID NO:8, respectively; SEQ ID NO:9 and SEQ ID NO: 10, respectively; SEQ ID NO: 11 and SEQ ID NO: 12; respectively; SEQ ID NO: 13 and SEQ ID NO: 14, respectively; SEQ ID NO: 15 and SEQ ID NO: 16, respectively; SEQ ID NO: 17 and SEQ ID NO: 18, respectively; SEQ ID NO: 19 and SEQ ID NO:20, respectively; SEQ ID NO:21 and SEQ ID NO:22, respectively; and SEQ ID NO:23 and SEQ ID NO:24, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided are methods comprising administering a FOLR1 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in the sets of amino acid sequences selected from (i) SEQ ID NO:25, SEQ ID NO:26, SEQ ID NO:27, SEQ ID NO:28, SEQ ID NO:29 and SEQ ID NO:30, respectively; and (ii) SEQ ID NO:31, SEQ ID NO:26, SEQ ID NO:32, SEQ ID NO:33, SEQ ID NO:34 and SEQ ID NO:35, respectively. In some embodiments, each VH and VL region comprises a humanized framework region. In some embodiments, each VH and VL region comprises a human framework region.

In some embodiments, provided are methods comprising administering a FOLR1 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in (i) SEQ ID NO:25, SEQ ID NO:26, SEQ ID NO:27, SEQ ID NO:28, SEQ ID NO:29 and SEQ ID NO:30, respectively. In some embodiments, each VH and VL region comprises a humanized framework region. In some embodiments, each VH and VL region comprises a human framework region.

In some embodiments, provided are methods comprising administering a FOLR1 antibody or antigen-binding portion thereof or other binding agent or conjugate thereof comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having the amino acids sequences set forth in SEQ ID NO:31, SEQ ID NO:26, SEQ ID NO:32, SEQ ID NO:33, SEQ ID NO:34 and SEQ ID NO:35, respectively. In some embodiments, each VH and VL region comprises a humanized framework region. In some embodiments, each VH and VL region comprises a human framework region.

In some embodiments, the subject is in need of treatment for a cancer and/or a malignancy. In some embodiments, the subject is in need of treatment for a FOLR1+ cancer or a FOLR1+ malignancy, such as for example, lung cancer, non-small cell lung cancer, ovarian cancer, breast cancer, uterine cancer, cervical cancer, endometrial cancer, pancreatic cancer, and renal cell cancer. In some embodiments, the method is for treating a subject having a FOLR1+ cancer or malignancy. In some embodiments, the method is for treating lung cancer in a subject. In some embodiments, the method is for treating non-small cell lung cancer in a subject. In some embodiments, the method is for treating breast cancer in a subject. In some embodiments, the method is for treating ovarian cancer in a subject. In some embodiments, the method is for treating cervical cancer in a subject. In some embodiments, the method is for treating endometrial cancer in a subject. In some embodiments, the method is for treating renal cell cancer in a subject. In some embodiments, the method is for treating uterine cancer in a subject. In some embodiments, the method is for treating pancreatic cancer in a subject.

The methods described herein include administering a therapeutically effective amount of a FOLR1 binding antibody or antigen binding portion thereof or other binding agent or conjugate thereof to a subject having a FOLR1+ cancer or malignancy. As used herein, the phrases “therapeutically effective amount”, “effective amount” or “effective dose” refers to an amount of the FOLR1 antibody or antigen binding portion thereof or other binding agent or conjugate as described herein that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the cancer is a solid tumor. In some embodiments, the cancer is a solid tumor, including but not limited to, lung cancer, non-small cell lung cancer, ovarian cancer, breast cancer, uterine cancer, cervical cancer, endometrial cancer, pancreatic cancer, and renal cell cancer. In some embodiments, the cancer or malignancy is FOLR1-positive (FOLR1+). As used herein, the terms “FOLR1-positive” or “FOLR1+” are used to describe a cancer cell, a cluster of cancer cells, a tumor mass, or a metastatic cell that express FOLR1 on the cell surface (membrane-bound FOLR1). Some non-limiting examples of FOLR1-positive cancers include, for example, lung cancer, non-small cell lung cancer, ovarian cancer, breast cancer, uterine cancer, cervical cancer, endometrial cancer, pancreatic cancer, and renal cell cancer.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a FOLR1+ cancer and in need of treatment, but need not have already undergone treatment for the FOLR1+ cancer. In some embodiments, a subject can also be one who has not been previously diagnosed as having a FOLR1+ cancer in need of treatment. In some embodiments, a subject can be one who exhibits one or more risk factors for a condition or one or more complications related to a FOLR1+ cancer or a subject who does not exhibit risk factors. A “subject in need” of treatment for a FOLR1+ cancer particular can be a subject having that condition or diagnosed as having that condition. In other embodiments, a subject “at risk of developing” a condition refers to a subject diagnosed as being at risk for developing the condition, or at risk for developing the cancer again (e.g., a FOLR1+ cancer).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in FOLR1+ cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a FOLR1 binding antibody or antigen-binding portion thereof or other binding agent or conjugate as described herein or a nucleic acid encoding the FOLR1 antibody or antigen-binding portion thereof or other binding agent as described herein into a subject by a method or route which results in binding to the FOLR1 binding antibody or antigen binding portion thereof or other binding agent or conjugate to FOLR1+ cancer cells or malignant cells. Similarly, a pharmaceutical composition comprising a FOLR1 binding antibody or antigen-binding portion thereof or other binding agent or conjugate as described herein or a nucleic acid encoding the FOLR1 antibody or antigen-binding portion thereof or other binding agent as described herein disclosed herein can be administered by any appropriate route which results in an effective treatment in the subject.

The dosage ranges for a FOLR1 binding antibody or antigen binding portion thereof or binding agent or conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a FOLR1 binding antibody or antigen binding portion thereof or other FOLR1 binding agent or FOLR1 conjugate thereof can be administered in a unit dose. The term “unit dose” when used in reference to a pharmaceutical composition refers to physically discrete units suitable as unitary dosage for the subject, each unit containing a predetermined quantity of active material (e.g., a FOLR1 binding antibody or antigen binding portion thereof or other binding agent or conjugate thereof), calculated to produce the desired therapeutic effect in association with the required physiologically acceptable diluent, i.e., carrier, or vehicle.

In some embodiments, a FOLR1 binding antibody or an antigen binding portion thereof or other binding agent or conjugate thereof, or a pharmaceutical composition of any of these, is administered with an immunotherapy. As used herein, “immunotherapy” refers to therapeutic strategies designed to induce or augment the subject's own immune system to fight the cancer or malignancy. Examples of an immunotherapy include, but are not limited to, antibodies such as check point inhibitors.

In some embodiments, the immunotherapy involves administration of a checkpoint inhibitor. In some embodiments, an immune checkpoint inhibitor includes an agent that inhibits CTLA-4, PD-1, PD-L1, and the like. Suitable anti-CTLA-4 inhibitors include, for example, ipilimumab, tremelimumab, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B 1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin. Oncology, 22(145): Abstract No. 2505 (2004) (antibody CP-675206); Mokyr et al., Cancer Res, 58:5301-5304 (1998), U.S. Pat. Nos. 5,977,318, 6,682,736, 7,109,003, and 7,132,281.

Suitable anti-PD-1 inhibitors, include, for example, nivolumab, pembrolizumab, pidilizumab, MEDI0680, and combinations thereof. In other specific embodiments, anti-PD-L1 therapy agents include atezolizumab, BMS-936559, MEDI4736, MSB0010718C, and combinations thereof.

Suitable anti-PD-1 inhibitors include, for example, those described in Topalian, et al., Immune Checkpoint Blockade: A Common Denominator Approach to Cancer Therapy, Cancer Cell 27: 450-61 (Apr. 13, 2015), incorporated herein by reference in its entirety.

In some embodiments, the checkpoint inhibitor is Ipilimumab (Yervoy), Nivolumab (Opdivo), Pembrolizumab (Keytruda), Atezolizumab (Tecentriq), Avelumab (Bavencio), or Durvalumab (Imfinzi).

In some embodiments, provided is a method of improving treatment outcome in a subject receiving immunotherapy. The method generally includes administering an effective amount of an immunotherapy to the subject having cancer; and administering a therapeutically effective amount of a FOLR1 antibody, antigen binding portion, other binding agent or conjugate thereof or a pharmaceutical composition thereof to the subject, wherein the FOLR1 antibody, antigen binding portion, other binding agent or conjugate thereof specifically binds to FOLR1+ cancer cells; wherein the treatment outcome of the subject is improved, as compared to administration of the immunotherapy alone. In some embodiments, the FOLR1 antibody, antigen binding portion, other binding agent or conjugate thereof comprises any of the embodiments of FOLR1 antibodies, antigen binding portions, other binding agents or conjugates thereof as described herein. In some embodiments, the binding agent is an antibody or an antigen-binding portion thereof. In some embodiments, the binding agent is a monoclonal antibody, a Fab, a Fab′, an F(ab′), an Fv, a scFv, a single domain antibody, a diabody, a bi-specific antibody, or a multi-specific antibody. In some embodiments, the binding agent is a conjugate of a FOLR1 monoclonal antibody, a Fab, a Fab′, an F(ab′), an Fv, a scFv, a single domain antibody, a diabody, a bi-specific antibody, or a multi-specific antibody.

In some embodiments, an improved treatment outcome is an objective response selected from stable disease, a partial response or a complete response as determined by standard medical criteria for the cancer being treated. In some embodiments, an improved treatment outcome is reduced tumor burden. In some embodiments, the improved treatment outcome is progression-free survival or disease-free survival.

The present invention is further illustrated by the following embodiments which should not be construed as limiting.
1. A binding agent comprising:
   a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having amino acids sequences selected from the sets of amino acid sequences set forth in the group consisting of:
   SEQ ID NO:25, SEQ ID NO:26, SEQ ID NO:27, SEQ ID NO:28, SEQ ID NO:29 and SEQ ID NO:30, respectively; and
   SEQ ID NO:31, SEQ ID NO:26, SEQ ID NO:32, SEQ ID NO:33, SEQ ID NO:34 and SEQ ID NO:35, respectively.
2. The binding agent of embodiment 1, wherein the VH and VL regions have amino acid sequences that are selected from the pairs of amino acid sequences set forth in the group consisting of:
   SEQ ID NO:1 and SEQ ID NO:2, respectively;
   SEQ ID NO:3 and SEQ ID NO:4, respectively;
   SEQ ID NO:5 and SEQ ID NO:6, respectively;
   SEQ ID NO:7 and SEQ ID NO:8, respectively;
   SEQ ID NO:9 and SEQ ID NO: 10, respectively;
   SEQ ID NO:11 and SEQ ID NO: 12; respectively;
   SEQ ID NO: 13 and SEQ ID NO: 14; respectively;
   SEQ ID NO: 15 and SEQ ID NO: 16; respectively;
   SEQ ID NO: 17 and SEQ ID NO: 18; respectively;
   SEQ ID NO: 19 and SEQ ID NO:20; respectively;
   SEQ ID NO:21 and SEQ ID NO:22; respectively; and
   SEQ ID NO:23 and SEQ ID NO:24; respectively;
   wherein the heavy and light chain framework regions are optionally modified with from 1 to 8 amino acid substitutions, deletions or insertions in the framework regions.
3. The binding agent of embodiment 1 or 2, wherein the VH and VL regions have amino acid sequences that are selected from the pairs of amino acid sequences set forth in the group consisting of:
   SEQ ID NO: 1 and SEQ ID NO:2, respectively;
   SEQ ID NO:3 and SEQ ID NO:4, respectively;
   SEQ ID NO:5 and SEQ ID NO:6, respectively;
   SEQ ID NO:7 and SEQ ID NO:8, respectively;
   SEQ ID NO:9 and SEQ ID NO: 10, respectively;
   SEQ ID NO: 11 and SEQ ID NO: 12; respectively;
   SEQ ID NO: 13 and SEQ ID NO: 14; respectively;
   SEQ ID NO: 15 and SEQ ID NO: 16; respectively;
   SEQ ID NO: 17 and SEQ ID NO:18; respectively;
   SEQ ID NO: 19 and SEQ ID NO:20; respectively;
   SEQ ID NO:21 and SEQ ID NO:22; respectively; and
   SEQ ID NO:23 and SEQ ID NO:24; respectively.
4. The binding agent of any of the preceding embodiments, wherein the VH and VL regions have amino acid sequences that are selected from the pairs of amino acid sequences set forth in the group consisting of:
   SEQ ID NO:3 and SEQ ID NO:4, respectively;
   SEQ ID NO:7 and SEQ ID NO:8, respectively;
   SEQ ID NO:9 and SEQ ID NO: 10, respectively;
   SEQ ID NO: 11 and SEQ ID NO: 12; respectively;
   SEQ ID NO: 15 and SEQ ID NO: 16; respectively;
   SEQ ID NO: 17 and SEQ ID NO: 18; respectively;
   SEQ ID NO: 19 and SEQ ID NO:20; respectively; and
   SEQ ID NO:21 and SEQ ID NO:22; respectively.
5. The binding agent of any of the preceding embodiments, wherein the VH and VL regions have amino acid sequences that are selected from the pairs of amino acid sequences set forth in the group consisting of:
   SEQ ID NO:3 and SEQ ID NO:4, respectively;
   SEQ ID NO:7 and SEQ ID NO:8, respectively; and
   SEQ ID NO:21 and SEQ ID NO:22; respectively.
6. The binding agent of embodiment 1, wherein the framework regions are human framework regions.
7. The binding agent of any of embodiments 1 to 6, wherein the binding agent is an antibody or an antigen-binding portion thereof.
8. The binding agent of any of the preceding embodiments, wherein the binding agent is a monoclonal antibody, a Fab, a Fab′, an F(ab′), an Fv, a scFv, a single domain antibody, a diabody, a bi-specific antibody, or a multi-specific antibody.
9. The binding agent of any of the preceding embodiments, wherein the heavy chain variable region further comprises a heavy chain constant region.
10. The binding agent of embodiment 7, wherein the heavy chain constant region is of the IgG isotype.
11. The binding agent of embodiment 10, wherein the heavy chain constant region is an IgG1 constant region.
12. The binding agent of embodiment 10, wherein the heavy chain constant region is an IgG4 constant region.
13. The binding agent of embodiment 11, wherein the IgG1 constant region has the amino acid sequence set forth in SEQ ID NO:39.
14. The binding agent of any of the preceding embodiments, wherein the light chain variable region further comprises a light chain constant region.
15. The binding agent of embodiment 14, wherein the light chain constant region is of the kappa isotype.
16. The binding agent of embodiment 15, wherein the light chain constant region has the amino acid sequence set forth in SEQ ID NO:40.
17. The binding agent of any of embodiments 9 to 16, wherein the heavy chain constant region further comprises at least amino acid modification that decreases binding affinity to human FcgammaRIII.
18. The binding agent of any of the preceding embodiments, wherein the binding agent is mono-specific.
19. The binding agent of any of embodiments 1 to 18, wherein the binding agent is bivalent.
20. The binding agent of any of embodiments 1 to 17, wherein the binding agent is bispecific.
21. A pharmaceutical composition comprising the binding agent of any of embodiments 1 to 20 and a pharmaceutically acceptable carrier.
22. A nucleic acid encoding the binding agent of any of embodiments 1 to 20.
23. A vector comprising the nucleic acid of embodiment 22.
24. A cell line comprising the vector of embodiment 22 or the nucleic acid of embodiment 21.
25. A conjugate comprising:
   the binding agent of any of embodiments 1 to 20,
   at least one linker attached to the binding agent; and
   at least one drug attached to each linker.
26. The conjugate of embodiment 25, wherein each drug is selected from a cytotoxic agent, an immunomodulatory agent, a nucleic acid, a growth inhibitory agent, a PROTAC, a toxin and a radioactive isotope.
27. The conjugate of any of embodiments 25 to 26, wherein each linker is attached to the binding agent via an interchain disulfide residue, a lysine residue, an engineered cysteine residue, a glycan, a modified glycan, an N-terminal residue of the binding agent or a polyhistidine peptide attached to the binding agent.
28. The conjugate of any of embodiments 25 to 27, wherein the average drug loading of the conjugate is from about 1 to about 8, about 2, about 4, about 6, about 8, about 10, about 12, about 14, about 16, about 3 to about 5, about 6 to about 8 or about 8 to about 16.
29. The conjugate of any of embodiments 25 to 28, wherein the drug is a cytotoxic agent. 30. The conjugate of embodiment 29, wherein the cytotoxic agent is selected from the group consisting of an auristatin, a maytansinoid, a camptothecin, a duocarmycin or a calicheamicin.
31. The conjugate of embodiment 30, wherein the cytotoxic agent is an auristatin.
32. The conjugate of embodiment 31, wherein the cytotoxic agent is MMAE or MMAF.
33. The conjugate of embodiment 30, wherein the cytotoxic agent is a camptothecin.
34. The conjugate of embodiment 33, wherein the cytotoxic agent is exatecan.
35. The conjugate of embodiment 33, wherein the cytotoxic agent is SN-38.
36. The conjugate of embodiment 30, wherein the cytotoxic agent is a calicheamicin.
37. The conjugate of embodiment 30, wherein the cytotoxic agent is a maytansinoid. 38. The conjugate of embodiment 37, wherein the maytansinoid is maytansine, maytansinol or a maytansine analog in DM1, DM3 and DM4, or ansamatocin-2.
39. The conjugate of any of embodiments 25 to 38, wherein the linker comprises mc-VC-PAB, CL2, CL2A or (Succinimid-3-yl-N)—(CH2)n-C(═O)-Gly-Gly-Phe-Gly-NH—CH2-O—CH2-(C═O)—, wherein n=1 to 5.
40. The conjugate of embodiment 39, wherein the linker comprises mc-VC-PAB.
41. The conjugate of embodiment 39, wherein the linker comprises CL2A.
42. The conjugate of embodiment 39, wherein the linker comprises CL2.
43. The conjugate of embodiment 39, wherein the linker comprises (Succinimid-3-yl-N)—(CH2)n-C(═O)-Gly-Gly-Phe-Gly-NH—CH2-O—CH2-(C═O)—.
44. The conjugate of embodiment 43, wherein the linker is attached to at least one molecule of exatecan.
45. The conjugate of any of embodiments 25 to 28, wherein the drug is an immune modulatory agent.
46. The conjugate of embodiment 45, wherein the immune modulatory agent is selected from the group consisting of a TRL7 agonist, a TLR8 agonist, a STING agonist, or a RIG-I agonist.
47. The conjugate of embodiment 46, wherein the immune modulatory agent is an TLR7 agonist.
48. The conjugate of embodiment 47, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3.
49. The conjugate of embodiment 46, wherein the immune modulatory agent is a TLR8 agonist.
50. The conjugate of embodiment 49, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.
51. The conjugate of embodiment 46, wherein the immune modulatory agent is a STING agonist.
52. The conjugate of embodiment 46, wherein the immune modulatory agent is a RIG-I agonist.
53. The conjugate of embodiment 52, wherein the RIG-I agonist is selected from KIN1148, SB-9200, KIN700, KIN600, KIN500, KIN100, KIN101, KIN400 and KIN2000.
54. The conjugate of any of embodiments 45 to 53, wherein the linker is selected from the group consisting of mc-VC-PAB, CL2, CL2A and (Succinimid-3-yl-N)—(CH2)n-C(═O)-Gly-Gly-Phe-Gly-NH—CH2-O—CH2-(C═O)—, wherein n=1 to 5.
55. A pharmaceutical composition comprising the conjugate of any of embodiments 25 to 54 and a pharmaceutically acceptable carrier.
56. A method of treating a FOLR1+ cancer, comprising administering to a subject in need thereof a therapeutically effective amount of the binding agent of any of embodiments 1 to 20, the conjugate of any of embodiments 25 to 54 or the pharmaceutical composition of embodiments 21 or 55.
57. The method of embodiment 56, wherein the FOLR1+ cancer is a solid tumor.
58. The method of embodiment 57, wherein the FOLR1+ cancer is selected from lung cancer, non-small cell lung cancer, ovarian cancer, breast cancer, uterine cancer, cervical cancer, endometrial cancer, pancreatic cancer, and renal cell cancer. 59. The method of any of embodiments 56 to 58, further comprising administering an immunotherapy to the subject.
60. The method of embodiment 59, wherein the immunotherapy comprises a checkpoint inhibitor.
61. The method of embodiment 60, wherein the checkpoint inhibitor is selected from an antibody that specifically binds to human PD-1, human PD-L1, or human CTLA4.
62. The method of embodiment 61, wherein the checkpoint inhibitor is pembrolizumab, nivolumab, cemiplimab or ipilimumab.
63. The method of any of embodiments 56 to 62, further comprising administering chemotherapy to the subject.
64. The method of any of embodiments 56 to 63, comprising administering the conjugate of embodiments 25 to 54 or the pharmaceutical composition of clam 55.
65. The method of any of embodiments 56 to 64, wherein the binding agent, conjugate or pharmaceutical composition is administered intravenously.
66. The method of embodiments 6, wherein the binding agent, conjugate or pharmaceutical composition is administered in a dose of about 0.1 mg/kg to about 12 mg/kg.
67. The method of any of embodiments 56 to 66, wherein a treatment outcome of the subject is improved.
68. The method of embodiment 67, wherein the improved treatment outcome is an objective response selected from stable disease, a partial response or a complete response.
69. The method of embodiment 67, wherein the improved treatment outcome is reduced tumor burden.
70. The method of embodiment 67, wherein the improved treatment outcome is progression-free survival or disease-free survival.
71. Use of the binding agent of any of embodiments 1 to 20 or the pharmaceutical composition of embodiment 21 for the treatment of FOLR1+ cancer in a subject.
72. Use of the conjugate of any of embodiments 25 to 54 or the pharmaceutical composition of embodiment 55 for the treatment of FOLR1+ cancer in a subject.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1: Generation of Human Antibodies Against Human FOLR1

Antibodies targeting human FOLR-1 were screened using a fully human antibody library. This library is a semisynthetic human antibody library in which the Fab was displayed on the surface of phage.

A standard protocol was followed for the library panning. Specifically, PolySorp or MaxiSorp Nunc-Immuno Tubes (Nunc-MG Scientific) were coated with 0.5 ml of human FOLR1 (ACRO-FO1-H52H1) antigen at 6 μg/ml (refer to the panning summary, Table 1), and placed in a refrigerator overnight. The tube was washed once with PBS, blocked with 1% BSA/PBS, and placed at RT (room temperature) for 1 hour. The tube was incubated with the library phage sample at indicated amount (CFU, refer to the panning summary, Table 1) at RT for 1 hour. The tube was washed 10 times with PBST buffer. To elute the bound phage, 0.5 ml of 100 mM TEA (triethylamine) was added, incubated at RT for 2 mins, and the eluate was transferred to a new tube and neutralized immediately by adding 0.25 ml of 1.0 M Tris-HCL, pH 8.0, with mixing. The eluant (0.75 ml) was added into 10 ml of exponentially growing E. coli TG1 (OD600˜0.5), mixed well and incubated without shaking at 37° C. (water bath) for 30 min. 10-fold dilutions of the culture were made in 2×TY media and 10 μl of each dilution was plated on TYE/amp/glu plates and incubated at 30° C. overnight. The next day, the colony number for each dilution was counted, and the CFU (colony form unit) for the panning output was calculated. The remaining culture was centrifuged at 2,800 g for 15 min, resuspended in 0.5 ml of 2×TY media, plated on two 150 mm TYE/amp/glu plates, and incubated at 30° C. overnight. The next day, 3-5 ml of 2×TY/amp/glu media was added to each plate and the bacteria were scraped from the plate with a cell spreader. Glycerol stocks were made by mixing 1.5 ml of bacteria and 0.5 ml of 80% glycerol and the stock placed at −80° C.

To prepare phage particles for the next round of selection, the glycerol stocks were inoculated into 40 ml of 2×TY/amp/glu media, starting at OD600˜0.01-0.05. The cultures were grown at 37° C. with shaking (300 rpm) until the OD600 reached 0.4-0.6. The cultures were infected by adding helper phage CM13 to the culture at a helper phage:bacteria ratio of 5-10:1. The cultures were incubated at 37° C. for 30 minutes while standing in a water bath with occasional mixing followed by shaking at 37° C. for 30 minutes. The bacterial cultures was centrifuged at 3000 rpm for 20 minutes and the supernatants were removed. The pellets were resuspended in 100 ml of 2×TY/amp/kan and then grown with shaking at 30° C. for overnight. The cultures were harvested by centrifuging at 6,000 g for 30 min. The phage particles were precipitated by adding 1/5 volume of PEG solution into the supernatant followed by 1 h incubation on ice followed by centrifuging at 4,000 g for 20 min at 4° C. The supernatants were discarded thoroughly. The phage pellets were resuspended in 1-2 ml of cold PBS. The residual bacteria were removed by micro-centrifugation at top speed for 5 min at 4° C. The phage prepared in this manner can be used immediately for selection, or stored at −80° C. in aliquots with 10% glycerol. The titer of the phage preparations were determined by infecting 100 ul of exponentially growing E. coli TG1 with a 10-fold dilution of the phage solution (in 2×TY, down to 10⁻¹¹). The selection was repeated starting with step 1 for a total round of 3˜4 rounds.

A total of 4 rounds of panning were performed. The concentration of the washing buffer PBS-Tween20 in the 2nd, 3rd and 4th rounds was gradually increased to 0.2%, 0.3%, and 0.4%, respectively.

After 4 rounds of screening, the target positive enrichment rate reached 1.5×10⁴, with a significant difference from the blank control, as shown in Table 1. Clones from two 96-well plates were picked for Phage ELISA validation; those clones with high binding with FOLR-1 were selected to sequenced.

In total, sixty-nine clones were sequenced and 12 unique VH sequences were obtained. These 12 VH sequences were analyzed and had 2 unique sets of HCDR3, as showed in Tables 2 and 4. For the clones with the 12 unique VH sequences, the VL sequences were then determined. Two unique VL sequences were obtained with two groups of unique LCDR3, as show in Tables 3 and 4.

Further analysis of the clone sequences using the Kabat system of CDR region showed that clones F1/8/9/26/48/50/100/112/123/131/138 have the same HCDRs and LCDRs, but with different heavy chain frame work (HFR) and light chain frame work (LFR) sequences, as shown in Table 5. Clone F40 has a different HCDRs and LCDRs, and with different HFR and LFR, as shown in Table 5.

TABLE 1
Process monitoring of the 4 rounds panning

| | | | | Enriching |
|---|---|---|---|---|
| Round | Conditions | Input | Output | factor |
| 1^{st} | Target protein: 200 nM (~6 ug/ml) Human FOLR1 | 1.0 × 10¹³ | 2.5 × 10⁵ | 4.0 × 10⁷ |
| | Blocking: 2% M-PBS | | | |
| | Washing: 0.1% Tween20 PBST, 9 times | | | |
| | Elution: TEA | | | |
| | Pre counter select: 2% M-PBS | | | |
| 2^{nd-} | Target protein: 200 nM (~6 ug/ml) Human FOLR1 | 5.3 × 10¹² | 9.6 × 10⁴ | 5.5 × 10⁷ |
| Positive | Blocking: 2% M-PBS | | | |
| Screening | Washing: 0.2% Tween20 PBST, 9 times | | | |
| | Elution: TEA | | | |
| | Pre counter select: 2% M-PBS | | | |
| 2^{nd}- | Target protein: no coating | 6.7 × 10¹¹ | 3.0 × 10⁴ | 2.2 × 10⁷ |
| Negative | Blocking: 2% M-PBS | | | |
| Screening | Washing: 0.2% Tween20 PBST, 9 times | | | |
| | Elution: TEA | | | |
| | Pre counter select: 2% M-PBS | | | |
| 3^{rd-} | Target protein: 200 nM (~6 ug/ml) Human FOLR1 | 5.3 × 10¹² | 3.0 × 10⁷ | 1.8 × 10⁵ |
| Positive | Blocking: 2% M-PBS | | | |
| Screening | Washing: 0.3% Tween20 PBST, 9 times | | | |
| | Elution: TEA | | | |
| | Pre counter select: 2% M-PBS | | | |
| 3^{rd}- | Target protein: no coating | 6.7 × 10¹¹ | 2.1 × 10⁵ | 3.2 × 10⁶ |
| Negative | Blocking: 2% M-PBS | | | |
| Screening | Washing: 0.3% Tween20 PBST, 9 times | | | |
| | Elution: TEA | | | |
| | Pre counter select: 2% M-PBS | | | |
| 4^{th-} | Target protein: 200 nM (~6 ug/ml) Human FOLR1 | 5.1 × 10¹² | 3.3 × 10⁸ | 1.5 × 10⁴ |
| Positive | Blocking: 2% M-PBS | | | |
| Screening | Washing: 0.4% Tween20 PBST, 9 times | | | |
| | Elution: TEA | | | |
| | Pre counter select: 2% M-PBS | | | |
| 4^{th}- | Target protein: no coating | 6.4 × 10¹¹ | 1.4 × 10⁵ | 4.6 × 10⁶ |
| Negative | Blocking: 2% M-PBS | | | |
| Screening | Washing: 0.4% Tween20 PBST, 9 times | | | |
| | Elution: TEA | | | |
| | Pre counter select: 2% M-PBS | | | |

TABLE 2
VH grouping and ranking

| | VH | HCDR3 |
|---|---|---|
| Clones | Grouping | Grouping |
| F8, 24 | VH-1 | HCDR3-A |
| F9 | VH-2 | |
| F26 | VH-3 | |
| F48 | VH-4 | |
| F50 | VH-5 | |
| F100 | VH-6 | |
| F112 | VH-7 | |
| F123 | VH-8 | |
| F131, 139 | VH-9 | |
| F138 | VH-10 | |
| F1, 14, 16, 30, 31, 32, 37, 45, 58, | VH-11 | |
| 74, 75, 76, 77, 78, 81, 82, 86, 87, | | |
| 88, 89, 92, 93, 95, 96, 97, 98, 99, | | |
| 103, 105, 106, 108, 111, 113, 114, | | |
| 115, 124, 130, 140, 146, 147, 148, | | |
| 153, 154, 162, 163, 164, 169, 170 | | |
| F40 | VH-12 | HCDR3-B |

TABLE 3
VL grouping and ranking

| | VL | LCDR3 |
|---|---|---|
| Clones | Grouping | Grouping |
| F1, 8, 9, 26, 48, 50, 100, 112, 123, 131, 138 | VL-1 | LCDR3-A |
| F40 | VL-2 | LCDR3-B |

TABLE 4
Variable region sequence of anti-FOLR-1 antibodies

| Clone Seq. | VH | VL |
|---|---|---|
| F1 | EVQLLESGGGVVQPGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRDNSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 2) |
| | QGTQVTVSS (SEQ 1) | |
| F8 | EVQLLESGGGVVQHGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRDNSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 4) |
| | QGTQVTVSS (SEQ 3) | |
| F9 | EVQLLESGGGVVQLGGPDSPVQPLDSPFSSYGMHWVRQ | EIVMTQSPSSVSASVGDRVAITCRASQGISSWLAWYQQKPGK |
| | APGKGLEWVAVISYDGSNKYYADSVKGRFTISRDNSKN | APKLLIYAASSLQSGVPSRFSGSGSGTDFTLTISSLQPEDFA |
| | TLYLQMNSLRAEDTAVYYCARPRAYYGAYGSSFDYWGQ | TYYCQQSYSTPLTFGGGTKVDIK (SEQ 6) |
| | GTQVTVSS (SEQ 5) | |
| F26 | EVQLLESGGGVVQRGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRDNSK | APKLLIYAASSLQSGVPSRFSGSGSGTDFTLTISSLQPEDFA |
| | NTLYLPMNSLRAEDTAVYYCARPRAYYGAYGSSFDYWG | TYYCQQSYSTPLTFGGGTKVDIK (SEQ 8) |
| | QGTQVTVSS (SEQ 7) | |
| F40 | EVQLLESGGGVVQPGRSLRLSCAASGFTFSSYAMHWVR | DIQVTQSPSSLSASLGDTVSIRCRASRGLTDSVAWYQQKPGQ |
| | QAPGKGLEWVAVISYDGSNKYYADSVKGRFIISRDNSK | APKLLIYAASTLQSGVPSRFGGSGSGSYFTLTITSLQPEDVA |
| | NTVYLQMNSLRAEDTAVYYCARPTYVFTYTGSSFDYWG | TYYCQNYKSAPWTFGQGTKVEIK (SEQ 10) |
| | QGTQVTVSS (SEQ 9) | |
| F48 | EVQLLESGGGVVQPGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRDNSK | APKLLIYAASSLQSGVPSRFSGSGSGTDFTLTISSLQPEDFA |
| | NTLYLHMNSLRAEDTAVYYCARPRAYYGAYGSSFDYWG | TYYCQQSYSTPLTFGGGTKVDIK (SEQ 12) |
| | QGTQVTVSS (SEQ 11) | |
| F50 | EVQLLESGGGVVQRGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRDNSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 14) |
| | QGTQVTVSS (SEQ 13) | |
| F100 | EVQLLESGGGVVQPGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRPNSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 16) |
| | QGTQVTVSS (SEQ 15) | |
| F112 | EVQLLESGGGVVQPGRSLRLSCAASGFTFSSYGMHWVR | EIVMTQSPSSVSASVGDRVAITCRASQGISSWLAWYWWKPGK |
| | QAPGKGLEWVAVISYDGSNKYYADSVKGRFTISRHNSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 18) |
| | QGTQVTVSS (SEQ 17) | |
| F123 | EVQLLESGGGVVQPERSLRLSCAASGFTFSSYGMHWVR | EIVMTQSPSSVSASVGDRVAITCRASQGISSWLAWYQQKPGK |
| | QAPGKGLEWVAVISYGDSNKYYADSVKGRFTISRANSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 20) |
| | QGTQVTVSS (SEQ 19) | |
| F131 | EVQLLESGGGVVQPGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISRANSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 22) |
| | QGTQVTVSS (SEQ 21) | |
| F138 | EVQLLESGGGVVQPGRSLRLSCAASGFTFSSYGMHWVR | EIVMTQSPSSVSASVGDRVAITCRASQGISSWLAWYQQKPGK |
| | QAPGKGLEWVAVISYDGSNKYYADSVKGRFTISTHNSK | APKLLIYAASSLQSGVPSRFSGSGSGTDFTLTISSLQPEDFA |
| | NTLYLQMNSLRAEDTAVYYCARPRAYYGAYGSSFDYWG | TYYCQQSYSTPLTFGGGTKVDIK (SEQ 24) |
| | QGTQVTVSS (SEQ 23) | |

TABLE 5
CDR sequence of anti-FOLR-1 antibodies of the present invention

| Clone Seq. | HCDR1 | HCDR2 | HCDR3 | LCD R1 | LCD R2 | LCD R3 |
|---|---|---|---|---|---|---|
| F1 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F8 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F9 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F26 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F40 | SYAMH | VISYDGSNKYYADSVKG | PTYVFTYTGSSFDY | RASRGLTDSVA | AASTLQS | QNYKSAPW |
| | (SEQ 31) | (SEQ 26) | (SEQ 32) | (SEQ 34) | (SEQ 35) | (SEQ 36) |
| F48 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F50 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F100 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F112 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F123 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F131 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |
| F138 | SYGMH | VISYDGSNKYYADSVKG | PRAYYGAYGSSFDY | RASQGISSWLA | AASSLQS | QQSYSTPLT |
| | (SEQ 25) | (SEQ 26) | (SEQ 27) | (SEQ 28) | (SEQ 29) | (SEQ 30) |

### Example 2: Validation of Antibodies Produced by HEK293 Cells

After obtaining the sequences of the antibody clones (as described above), further analyses were done using the full IgG molecule of the. First, expression of full-length antibody molecules with an IgG1 Fc was performed in 48-well or 96-well microplates, and the supernatants were collected for the detection of expression levels and antigen or cell binding ability.

### 2.1 Antibody Expression in 48 or 96 Wells Plate.

The cDNA sequences encoding the heavy and light chains of antibodies F1, F8, F26, F40, F48, F50, F100, F112, F123, F131, and F138 were constructed to the vector PTT5. HEK293 cells were collected, adjusted to a cell density with 1×10⁶/ml, and plated into 48/96-well cell culture plates at 200 or 400 μL per well in a 37° C. incubator with 5% CO₂ for later use. For transfection in 96-well plates, 0.5 μg of plasmid was diluted in 20 μL OPTI medium, mixed well, and 2.5 μL of transfection reagent T1 (plasmid: T1=1:5) was diluted in 20 μL OPTI medium, mixed well, incubated at room temperature for 5 min. The transfection reagent T1 diluent was added to the DNA, mixed well, and incubated at room temperature for 30 min. The transfection complex was formed during the incubation. The transfection complex was added to the cells, mixed well, and incubated at 37° C. in a 5% CO₂ incubator for 48 hours. When transfecting in 48-well plates, the amount of plasmid and transfection reagent was doubled. On the second day after transfection, the supernatant was collected to detect the antibody bio-activity with ELISA or FACS.

### 2.2 IgG Expression Level.

Antibody expression levels in 96-well were tested by standard ELISA. Briefly, anti-Human IgG Fc antibody (Sigma, 18885-2ML) was diluted to 5 μg/ml with a carbonic acid coating solution at pH 9.6 and 100 μL was coated in each well of 96-well microtiter plate at 4° C. overnight. The liquid in the wells was discarded and the wells were washed three times with PBST, blocked with 4% skimmed milk powder-PBS (Sigma, D5652-1L), 300 μL/well, and incubated in 37° C. for 1 hour. The liquid in the wells was discarded, then the wells were washed with three times with PBS. Samples were added to the 96-well microtiter plate using 100 μL/well. PBS was added in the control group. The plates were incubated at 37° C. for 1 hour, then the liquid was discarded and the wells were washed with three times with PBST. HRP-goat anti-human IgG (Sigma, |18885-2ML) was added (1:5000 dilution) using 100 μL/well and the plates incubated at 37° C. for 1 hour. Then the liquid in the plates was discarded, and the plates were washed five times with PBST. A TMB solution was added using 100 μL/well. 2M H₂SO₄ was then added to each well using 50 μL per well to terminate the reaction after 10˜15 mins. The A450 values were read using a microplate reader. The results are shown in Table 6. All of the antibodies had a normal expression except clone F50.

### 2.3 Antibody Binding to Human and Cynomolgus FOLR1 Protein.

The ability of the antibodies to bind to human FOLR1 protein or to cross binding to cynomolgus FOLR1 protein was tested by standard ELISA. Briefly, the human FOLR1 (ACRO-FO1-H52H1) or cynomolgus FOLR1 protein (ACRO, F01-C52H8) with a His tag was diluted to 5 μg/ml with a carbonic acid coating solution at pH 9.6 and 100 μL antigen was coated in each well of 96-well microtiter plate at 4° C. overnight. The liquid in the wells was discarded and the wells were washed three times with PBST. The wells were then blocked with 4% skim milk powder-PBS (Sigma, D5652-1L), using 300 μL/well and the plates incubated in 37° C. for 1 hour. The liquid in the wells was discarded, and the well were washed three times with PBS. Samples were added using 100 ul/well; PBS was added in the control group. The plates were incubated at 37° C. for 1 hour. The liquid in the wells was discarded and the wells were washed three times with PBST. HRP-goat anti-human IgG (Sigma, 118885-2ML) was added (with 1:5000 dilution, 100 μL/well) and the plates were incubated in 37° C. for 1 hour. Then the liquid in the wells was discarded, and the wells were washed five times with PBST. A TMB solution was added using 100 μL/well. 2M H₂SO₄ was added to each well using 50 μL to terminate the reaction after 10˜15 mins. The A450 values were read using a microplate reader.

The expression levels of the IgGs in microtiter plates and the binding to human FOLR1 protein are shown in Table 6. All of the antibodies had a normal binding to human FOLR1 protein except clone F50.

The results of anti-FOLR1 antibody cross binding to cynomolgus FOLR1 protein is shown in Table 7. All of the antibodies had good cross binding to cynomolgus FOLR1 protein except clone F50.

### 2.4 Antibody Binding to Tumor Cell Lines Expressing High FOLR1.

The binding activity of the antibodies to Hela cells (ATCC® CCL-2, provided by COBIOER) and to RPTEC/TERT1 cells (ATCC® CRL-4031, provided by COBIOER) was tested by flow cytometry using the transfection supernatants. Briefly, target cells were digested with 0.02% EDTA-2Na, centrifuged at 1500 rpm for 3 mins, and resuspended with PBS. After counting, the cells were added to a 1.5 ml centrifuge tube with 1×10⁶ cells per tube, centrifuged at 1500 rpm for 5 minutes, and the supernatant was discarded. Then all the operations were carried out on an ice bath. 100 μL of transfection supernatant was added to each 1.5 ml centrifuge tube. Blank cells, blank cells plus secondary antibody, medium and HEK293 supernatant were set up as the controls. The reaction was performed on an ice bath for 1 hour. Then the cells were pelleted and washed twice with PBS. The secondary antibody, goat anti-Human IgG (PE, abcam, ab98596), was diluted (1:200) and added using 100 μL per tube. The reaction was performed on an ice bath for 1 hour in the dark. The cells were pelleted again and washed twice with PBS, resuspended in 300 μL PBS, and FL2 fluorescence readings were measured by cytometry. The results were analyzed by FlowJoTM10 software.

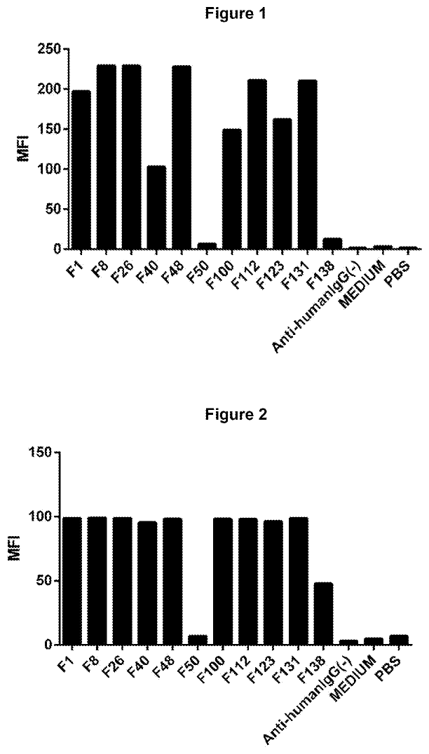

The results for anti-FOLR1 antibody binding to Hela cells are shown in FIG. 1. The results demonstrate that clone F50 was negative for Hela cell binding. The clones F40 and F138 were weakly positive for Hela cell binding. The remaining eight clones were positive for Hela cell binding.

The results of anti-FOLR1 antibody binding to RPTEC/TERT1 cells are shown in FIG. 2. The results demonstrate that clone F50 was negative for RPTEC/TERT1 cell binding. Clone F138 was weakly positive for RPTEC/TERT1 cell binding. The remaining 9 clones were positive for RPTEC/TERT1 cell binding.

TABLE 6
Comparison of antibody levels and
binding to human FOLR1 protein

| | OD450 value | | |
|---|---|---|---|
| | Target binding, | IgG level, coating | Target |
| | coating with Human | Anti-Human | binding/ |
| Samples | FOLR1 Protein | IgG Fc | IgG level |
| F40 | 2.3860 | 2.3350 | 1.022 |
| F123 | 2.3160 | 2.1930 | 1.056 |
| F1 | 2.4330 | 2.2540 | 1.079 |
| F8 | 2.4540 | 2.1900 | 1.121 |
| F131 | 2.3470 | 2.3090 | 1.016 |
| F50 | 0.1300 | 0.4930 | 0.264 |
| F112 | 2.3530 | 2.1010 | 1.120 |
| F48 | 2.5450 | 2.2740 | 1.119 |
| F26 | 2.4310 | 2.0100 | 1.209 |
| F100 | 2.2980 | 1.5550 | 1.478 |
| F138 | 2.4080 | 2.0060 | 1.200 |
| Medium | 0.0920 | 0.1050 | 0.876 |
| PBS | 0.0920 | 0.1430 | 0.643 |

TABLE 7
Comparison of antibody cross binding ability
to cynomolgus FOLR1 protein

| | OD450 | | | | | |
|---|---|---|---|---|---|---|
| | | | | Target: Cynomolgus/ | | |
| | Target: Human FOLR1 | | | Rhesus macaque FOLR1, | | |
| | Protein His Tag | | | Protein, His Tag | | |
| | Diluted | Diluted | Diluted | Diluted | Diluted | Diluted |
| Samples | 1× | 5× | 25× | 1× | 5× | 25× |
| F40 | 0.811 | 0.382 | 0.163 | 1.354 | 1.111 | 0.924 |
| F123 | 1.715 | 1.468 | 1.328 | 1.363 | 1.301 | 1.074 |
| F1 | 1.660 | 1.445 | 1.177 | 1.384 | 1.256 | 0.994 |
| F8 | 1.672 | 1.645 | 1.315 | 0.896 | 1.285 | 1.059 |
| F131 | 1.609 | 1.522 | 1.384 | 1.203 | 1.270 | 1.088 |
| F50 | 0.094 | 0.129 | 0.098 | 0.093 | 0.107 | 0.066 |
| F112 | 1.556 | 1.421 | 1.174 | 1.257 | 1.212 | 0.933 |
| F48 | 1.557 | 1.590 | 1.290 | 1.148 | 1.040 | 0.908 |
| F26 | 1.638 | 1.515 | 1.475 | 1.237 | 1.207 | 1.018 |
| F100 | 1.609 | 1.528 | 1.180 | 1.163 | 1.034 | 0.875 |
| F138 | 1.636 | 1.692 | 0.827 | 1.140 | 1.142 | 1.173 |
| Medium | 0.085 | 0.092 | 0.081 | 0.170 | 0.151 | 0.096 |

| | | |
|---|---|---|
| PBS | 0.082 | 0.081 |

### Example 3: Characterization of Anti-Human FOLR1 Antibodies Produced by HEK293 Cell Expression in Shaker Flask

The binding of the anti-FOLR1 antibodies was quantitatively studied by obtaining a sufficient amount of protein by expressing the antibodies in suspension cells. The plasmids were transfected into the suspension cells for expression. The supernatants were collected for antibody purification. Highly purity antibodies were used to quantitatively detect the binding and internalization of the antibody on tumor cells that had high FOLR1 protein levels.

### 3.1 Antibody Expression and Purification.

The plasmids encoding antibodies F8, F26, F40, F48, F100, F112, F123, and F131 were transfected into HEK293 cells. Briefly, HEK293 cells were collected, adjusted to a cell density with 1×10⁶/ml and cultured with 30 mL medium in 125 ml shaker flasks in a 37° C. shaker with 5% CO₂ for later use. For transfection, 30 μg of plasmid was diluted in 1500 ul KPM medium, mixed well, and 150 μL of transfection reagent T1 (plasmid:T1=1:5) was diluted in 1500 μL KPM medium, mixed well and incubated at room temperature for 5 min. The transfection reagent T1 diluent was added to the DNA, mixed well, and incubated at room temperature for 30 mins to form the transfection complex. The transfection complex was added to the cells, mixed well, and incubated at 37° C. in a 5% CO₂ shaker at 120 rpm for 48 hours. TN1 solution was added to a final concentration of 0.5% after 24 h. On the sixth day after transfection, the supernatant was collected and purified.

Antibody purification was carried out by a standard process using protein A or protein G. Briefly, each supernatant was filtered through a 0.22 μm filter membrane and loaded onto column equilibrated with binding buffer (PB, pH7.2). The column was washed with binding buffer until a stable baseline was obtained with no absorbance at 280 nm. Antibody was eluted with 0.1M citric acid buffer containing 0.15M NaCl, pH3.4, using a flow rate of 1 ml/min. Fractions of approximately 1.5˜3.5 ml were collected and neutralized by the addition of 10% volume of 1M Tris-HCl, pH9.0. Then the antibody samples were dialyzed overnight twice against 1×PBS and sterilized by filtering through a 0.2 μm filter membrane. The purity was tested using 12% SDS-PAGE.

The expression levels and purification result are shown in Table 8. Antibodies F8, F26 and F131 had higher expression levels while antibody F100 had the lowest expression level. All the antibodies had a high purity (data not shown).

TABLE 8
Comparison of antibody expression levels

| | | Con. | Vol. | Qua. |
|---|---|---|---|---|
| Host cell | Clone | (ug/ml) | (ml) | (mg) |
| HEK293 | F8 | 387.6 | 4.00 | 1.55 |
| HEK293 | F26 | 396.6 | 3.50 | 1.39 |
| HEK293 | F40 | 182.1 | 4.01 | 0.73 |
| HEK293 | F48 | 131.1 | 2.97 | 0.39 |
| HEK293 | F100 | 60.6 | 1.98 | 0.12 |
| HEK293 | F112 | 129.6 | 4.01 | 0.52 |
| HEK293 | F123 | 171.6 | 2.97 | 0.51 |
| HEK293 | F131 | 359.1 | 3.51 | 1.26 |

### 3.2 Antibody Binding to Tumor Cell Lines Having High FOLR1 Levels.

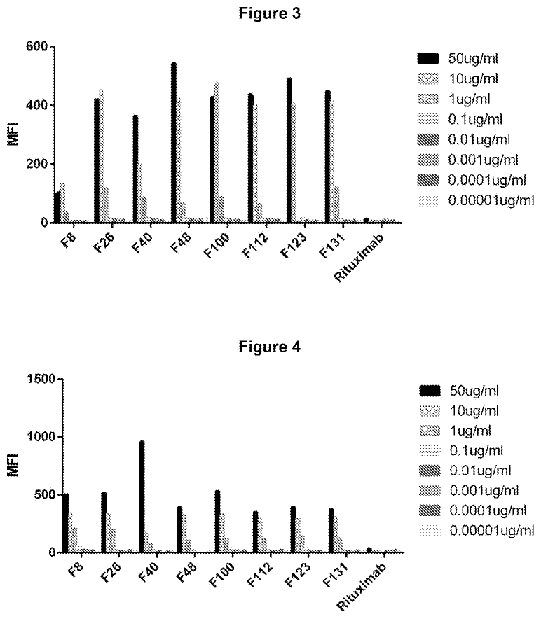

Anti-FOLR1 antibody binding to Hela and RPTEC/TERT1 cells was tested by FACS. The study was carried out as described above. The results are shown in FIGS. 3 and 4. All antibodies bind to Hela and to RPTEC/TERT1 cells in a dose-dependent.

### 3.3 Internalization Rate Characterization.

Anti-FOLR1 antibodies F8, F26, F40, F48, F100, F112, F123, and F131 were tested for the ability to internalize into the FOLR-1-expressing tumor cell lines Hela and RPTEC/TERT1 using a pHAb assay where the antibodies were labelled with pHAb fluorescent dye. Antibody labeling was performed according to the directions in the kit. Specifically, 50 μL of magnetic beads were added to a 1.5 ml EP tube. The EP tube was placed on a magnetic stand for 10 s and the protective solution over the magnetic beads was removed. Each tube of magnetic beads was washed with 250 μL PB and 100 μg antibody was added to each tube of magnetic beads (buffer system: citric acid/sodium Tris-HCl (pH 6.0)). The volume was made up to 1 ml with PB, and the reaction solution was mixed and rotated for 1 h at room temperature. Then the magnetic beads were washed with 250 μL PB, equilibrated with 250 μL NaHCO₃. 100 μL NaHCO₃ and 1.2 μL of prepared pHAb dye (prepared before use) was added to each tube and the reaction was placed for 1 h in the dark. Each tube was washed twice with 250 μL PB. 50 mM glycine was added to each tube using 100 μL at room temperature for 5 min and then labeled antibody was eluted. Then 2M Tris buffer was added to the elution for neutralization. The final labeled antibody was stored in the dark for later use.

Hela or RPTEC/TERT1 cells were seeded at 15,000 cells per well with 100 μL and cultured in a 5% CO₂ incubator at 37° C. for 20˜24 h. pHAb-labeled test antibodies were added to the wells at a concentration of 10 μg/ml. The plates were then read on a Thermo VARIOSKAN FLASH with an excitation wavelength of 520 nm and an absorption wavelength of 570 nm at 0 h, 1 h, 4 h, 6 h, and 23 h, respectively.

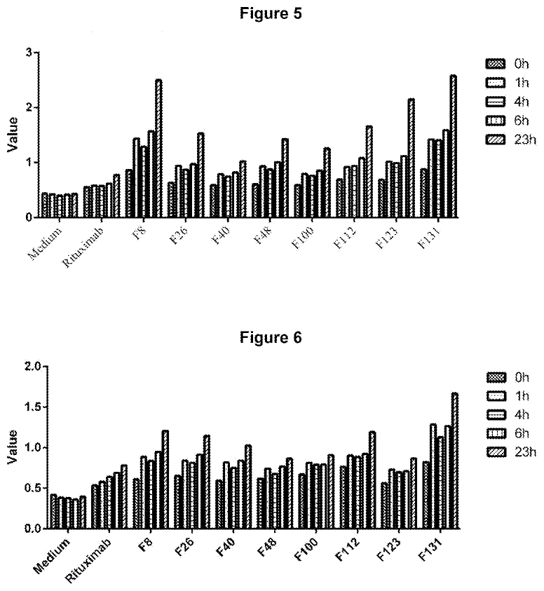

The results are shown in FIG. 5 and FIG. 6. All the anti-FOLR1 antibodies tested showed a time dependent increase in pHAb fluorescence in FOLR1-expressing Hela and RPTEC/TERT1 cells. This result indicates that each antibody internalized into Hela and RPTEC/TERT1 cells, with antibodies F8 and F131 having the most strongly internalization rate.

### Example 4: Characterization of Anti-FOLR-1 Immunoconjugates

Further characterization of the anti-FOLR-1 antibodies as immunoconjugates was performed.

### 4.1 Expression of Reference Antibody and Antibodies F8, F26 and F131

The ImmunoGen Inc. anti-FOLR-1 antibody mirvetuximab (huFR107) was used as a control. The amino acid sequences of the VH and VL regions of huFR107 were obtained from U.S. Pat. No. 8,557,966 (SEQ ID NOs:36 and 37, respectively) and were codon-optimized. The optimized cDNAs encoding huFR107 and encoding antibodies F8, F26 and F131 were constructed in the vector pcDNA3.4. Then the plasmids were transiently transected into ExpiCHO-S cells using a standard ExpiFectamine CHO Transfection procedure (Gibco, A29129) in Erlenmeyer flasks. The suspended transient transfections were incubated for 10 days and then the cleared supernatants were purified by a Protein A column and followed by SDS-PAGE as described above.

### 4.2 Preparation of Anti-FOLR-1 Immunoconjugates

The pH of antibody solution was adjusted within the range of pH 7.0˜7.5 by adding 0.5M sodium phosphate dibasic. The indicated amount of 0.5M EDTA was added to achieve a final EDTA concentration of 5 mM in the antibody solution. The indicated amount of 10 mM TCEP (Tris(2-chloroethyl) phosphate solution was added to achieve the desired TCEP/mAb molar ratio. The reduction reaction was placed at RT for 90 mins. Then DMSO was added to achieve a 10% v/v. The drug-linker mc-VC-PAB-MMAE was dissolved in DMSO to achieve a final concentration of 10 mM and the indicated amount was added in the reaction solution in a molar excess of 30-50% compared to the moles of cysteine thiols available. The conjugation reaction was placed at RT for 30 mins. NAC (N-Acetyl-L-cysteine) stock solution was added to achieve an NAC/Mc-VC-PAB-MMAE molar ratio of 5. The quenched reaction was placed at RT for 15 mins. The purification was carried out by PD10 column.

The purity of the anti-FOLR-1 immunoconjugates was assessed with size exclusion chromatography (SEC) on a TSK gel G3000SWXL, 7.8×300 mm column (Tosoh Bioscience) using the Waters HPLC E2695&2489 system. The operation was carried out at 25° C., using a mobile phase of 50 mM Na₂PO₄ (pH6.7) and 10% IPA, run with a flow rate of 0.8 mL/min over 20 min. Referring to Table 9, all four ADCs had high purity.

The hydrophobicity of the anti-FOLR-1 immunoconjugates were assessed with Hydrophobic interaction chromatography (HIC) on a Hydrophobic interaction TosoHaas TSK gel Butyl-NPR column (4.6 mm ID x 3.5 cm, with a particle size of 2.5 μm) using the Waters HPLC E2695&2489 system. Briefly, the HPLC system was operated at 25° ° C. with mobile phase A:50 mM Na₂PO₄/1.5 M (NH₄)₂SO₄ PH7.0 and mobile phase B: 50 mM Na₂PO₄/25% IPA, pH7.0. The mobile phases were filtered through a 0.22-μm membrane filter (Millipore), run with a flow rate of 0.5 mL, 30 min. The parameters of the linear gradient are shown in Table 10. The DARs (Drug antibody ratios) of the anti-FOLR-1 immunoconjugates were determined according to the HIC data and were within the range of 3˜4 (data not shown).

TABLE 9
Purity of anti-FOLR1 immunoconjugates

| | F8- | F26- | F131- | FR107- |
|---|---|---|---|---|
| | ADC | ADC | ADC | ADC |
| Purity (%) | 98% | 100% | 100% | 100% |

TABLE 10
Process for the
linear gradient

| Time/min | B/100% |
|---|---|
| 0.0 | 0 |
| 12.0 | 100 |
| 12.1 | 0 |
| 18.0 | 0 |

### Example 5: Binding Characterization of the Immunoconjugates

A comparison of anti-FOLR1 conjugate binding to FOLR1-his or a FOLR1 high expression tumor cell line was performed by standard ELISA or FACS.

### 5.1 ELISA Tests.

Recombinant His-tagged FOLR1 was coated on a 96-well micro plate (Thermo, cat:468667) in PBS at 2 μg/ml, 100 μL per well overnight. The coating solution was removed and the plate was washed twice by filling the wells with 350 μL/well TBST. The plate was blocked by adding 200 μL blocking buffer (2% BSA/TBST) per well. The plate was placed at 37° C. for 2 h. The plate was washed twice with 350 μL/well TBST. The samples were added at a starting concentration of 10 μg/ml and titrated down at 1:3 serial dilutions. The plate was placed at room temperature for 1 h. The solution was removed and washed twice with 350 μL/well TBST. Goat Anti-Human IgG Fc HRP (abcam, ab98624) was diluted in 1:20000 with the blocking buffer, and added to the plate at 100 μL per well. The plate was incubated at room temperature for 1 h. The plate was washed four times with 350 μL/well TBST. 100 μL of TMB (solution A: solution B, 1:1) solution was added in each well and the reaction was placed in dark for 3-10 mins. 50 μL of stopping solution (2M H₂SO₄) was added and the optical density at 450 nm and 630 nm was read. The data were analyzed with GraphPadPrism5 software.

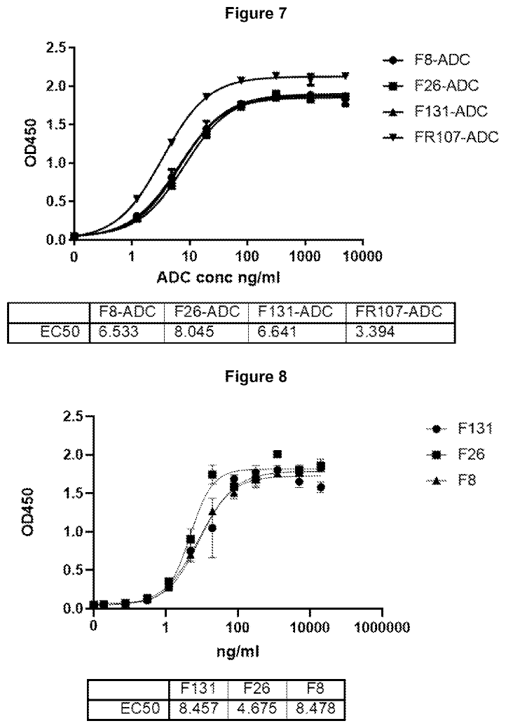

The ELISA results are shown in FIG. 7 and FIG. 8. The data showed that the activity of the conjugates in binding to target FOLR1 protein was not affected after conjugation, and there was no significant difference between the three ADCs in binding to the recombinant protein FOLR-1.

### 5.2 FACS Tests.

FOLR1-expressing Hela, OVCAR3 (ATCC® HTB-161™, provided by COBIOER), OV90 (ATCC® CRL-11732™, provided by COBIOER) and IGROV-1 (provided by COBIOER) cells were incubated with varying concentrations of the anti-FOLR-1 conjugates. Each antibody conjugate was incubated for 0.5 h in 0.1 ml FACS buffer (PBS supplemented with 0.1% BSA). Then, the cells were pelleted, washed, and incubated for 0.5 h with 0.1 ml of PE-conjugated goat anti-human IgG-antibody (Abcam, Ab98596). The cells were pelleted again, washed with PBS and resuspended in 100 μL PBS. Samples were analyzed using CytoFLEX (Beckman).

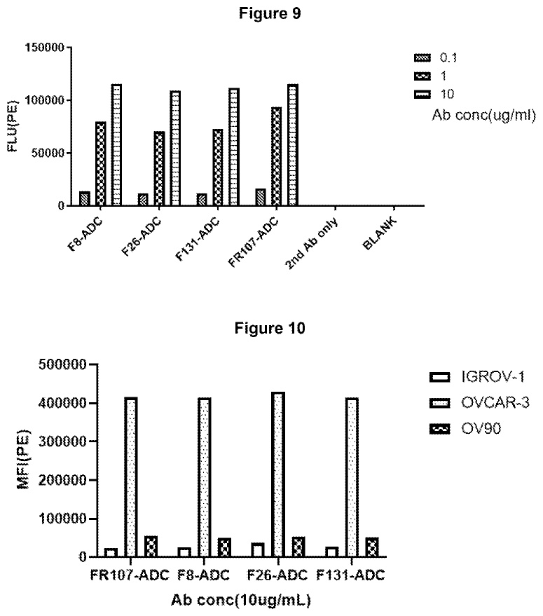

The results are shown in FIG. 9 and FIG. 10. There was no significant difference in binding to the cell lines by the three antibody conjugates as compared to the reference antibody conjugate. All three anti-FOLR1 conjugates had a stronger binding to OVCAR3 than binding to IGROV-1 or binding to OV90 cell lines.

### Example 6: Internalization of Anti-FOLR1 Immunoconjugates

Anti-FOLR1 conjugates (F8-ADC, F26-ADC, F131-ADC and control FR107-ADC) were tested for their ability to internalize into FOLR1-expressing Hela, OVCAR3, IGROV-1 and OV90 tumor cells using an immune-fluorescence staining assay.

Specifically, 3×10⁵ cells were harvested from a tissue culture flask by treatment with 0.25% Trypsin/EDTA, then incubated with each of the immunoconjugates at 10 microgram/ml in FACS buffer (1×PBS containing 0.1% BSA) at 4° C. for 30 min. A human IgG1 isotype control was used as a negative control. The cells were washed to remove the unbound material and incubated at 4° C. or shifted to 37° C. At the set time points (0 h, 4 h, 24 h), cells were stained with PE-conjugated anti human Fc antibody (Abcam, Ab98596) at 4° C. for 30 min and were analyzed by flow cytometry. The internalization ratio was determined by subtracting the 37° C. MFI from the 4° C. MFI, and then compared with 4° C. MFI.

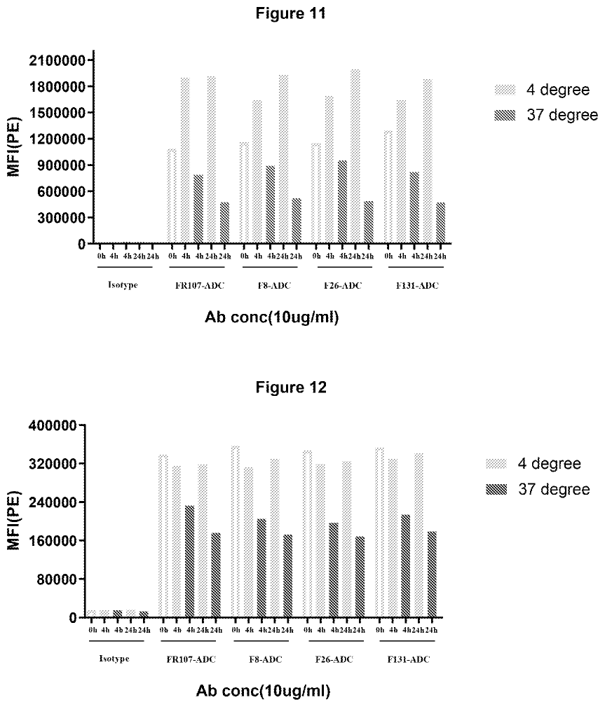

FIG. 11 and FIG. 12 show changes in surface levels of either immunoconjugates or the isotype control on Hela and OVCAR3 cell lines kept at 4° C. for the course of the 4 h or 24 h studies. Surface levels of the immunoconjugates declined significantly when cells shifted to 37° C. over the course of the assay. This observation suggests there were no significant differences in internalization on two tumor cell lines of the three tested anti-FOLR1 immunoconjugates, as well as the reference antibody conjugate.

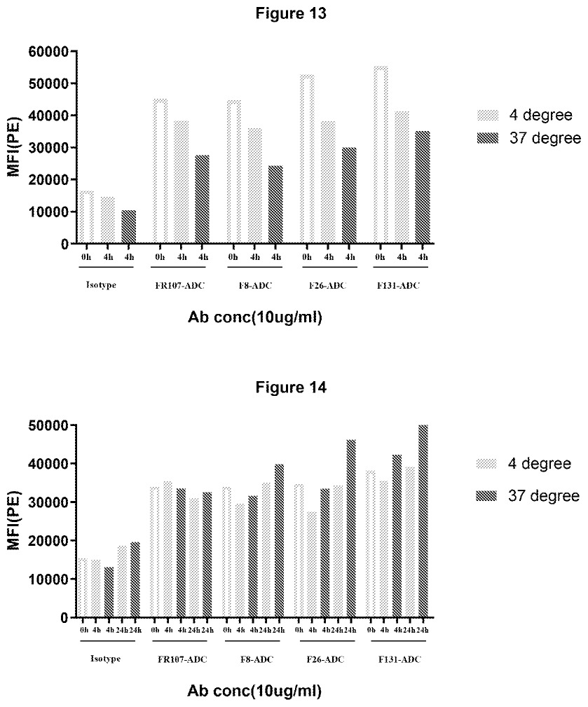

FIG. 13 shows the internalization result of anti-FOLR1 immunoconjugates on the tumor cell line OV90. The result showed that the internalization of the F8-ADC was better than that of the other ADCs on OV90 cell line. From the results displayed in FIG. 14, internalization on IGROV-1 tumor cell line could not be determined.

The internalization results of anti-FOLR1 conjugates on Hela, OVCAR3, and OV90 are summarized in Table 11.

TABLE 11
Internalization ratio (%) of anti−FOLR1 conjugates

| | | Isotype- | F8- | F26- | F131- | FR107- |
|---|---|---|---|---|---|---|
| Cell line | Time(h) | ADC | ADC | ADC | ADC | ADC |
| Hela | 4 | 0 | 45.7 | 43.3 | 50 | 58.4 |
| | 24 | 13.5 | 73.1 | 75.5 | 74.8 | 75.1 |
| OVCAR3 | 4 | 0.1 | 34.5 | 38.3 | 35.2 | 26.3 |
| | 24 | 20.5 | 47.6 | 48.1 | 47.7 | 44.8 |
| OV90 | 4 | 28.6 | 32.7 | 21.6 | 15 | 28 |

### Example 7: In Vitro Cytotoxicity Assay

The ability of the F8, F26, F131 and FR107 conjugates to inhibit cell growth was measured using in vitro cytotoxicity assays. The following method was used.

Cells were harvested and seeded into 96-well solid white flat bottom plates at the indicated amounts (according to the cell growth rate) prior to adding the anti-FOLR1 conjugates. Next day the cells were exposed to the conjugates at a drug range from 30 microgram/ml to 0.37 microgram/ml or from 100 microgram/ml to 0.015 microgram/ml, using 1:3 serial dilutions, with duplicate wells. The plates were incubated at 37° C. for 120 h. Then, 40 microL CTG (Promega, G7572) per well was added to the plates and the plates were read on a MD 13X reader after 5 min incubation. Growth inhibition was measured as a percent of growth relative to untreated cells using Microsoft Excel and Prism software.

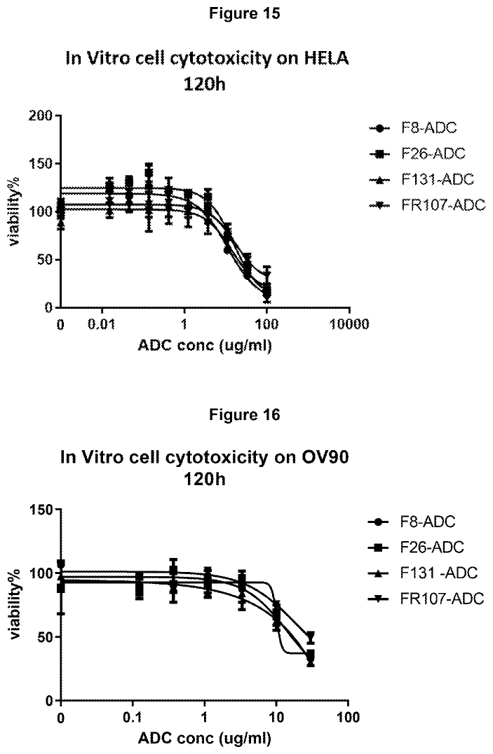

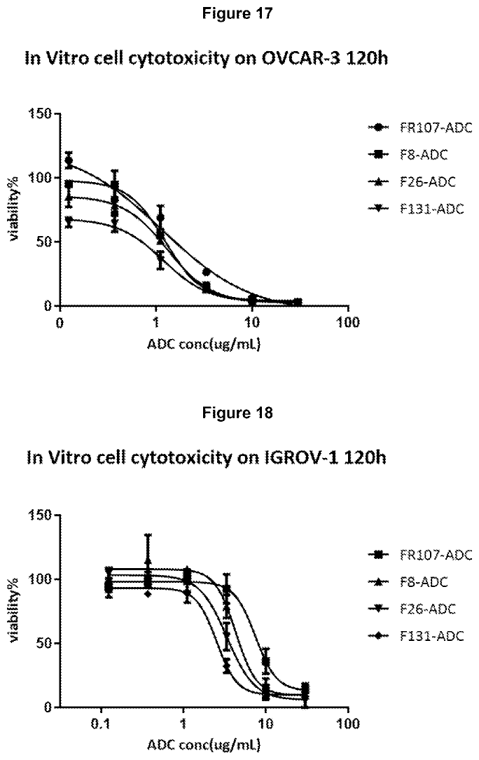

The results are show in FIGS. 15-18 and Table 12. All the 3 anti-FOLR1 conjugates (F8-ADC, F26-ADC and F131-ADC) had a slightly better cytotoxicity on Hela and IGROV-1 cells than the reference antibody conjugate (FR107-ADC), as shown in FIGS. 15 and 18. In addition, F131-ADC had somewhat better cell growth inhibitory activity than the F8-ADC and F26-ADC on IGROV-1 cell line.

TABLE 12
Cytotoxicity of anti-FOLR conjugates in vitro

| | IC50, ug/ml (n = 2) | | | |
|---|---|---|---|---|
| | F8- | F26- | F131- | FR107- |
| Cell line | ADC | ADC | ADC | ADC |
| Hela | 11.17 | 15.31 | 14.25 | 17.11 |
| OVCAR3 | 1.234 | 1.312 | 1.194 | 1.158 |
| IGROV | 4.301 | 3.382 | 2.523 | 7.460 |

### Example 8: Pharmacokinetic (PK) and Safety of Anti-FOLR-1 Immunoconjugates in a Mouse Model

### 8.1 PK

BALB/c normal mice were purchased from JOINN Laboratories (Suzhou) and used 1 week after housing. The mice were housed in groups in sterilized cages and maintained under pathogen-free conditions. In the experimental room, the environmental conditions were as follows: a temperature of 20° C.˜22° C. and humidity of 59%˜78% humidity, with artificial illumination for 12 h. The mouse cages were a polysulfone box, which were used after autoclaving, with the specification of 325 mm×210 mm×180 mm. Up to 5 animals were raised in each box, with the experiment number, experimental start time, project leader, experimental personnel, animal source, group and animal number indicated on the cage card. The experimental animals were ear-marked. The mice were fed an FR-2 diet and were provided tap-water (used after autoclaving). Their body weights were approximately 20-22 g at dosing.

4 groups with 6 mice per group were treated with single dose of F8, F26, F131 and FR107 immunoconjugates at 3 mg/kg intravenously (IV). Blood samples were collected at 10 min, 4 h, 1 d, 4 d, 7 d, 10 d, 14 d, and 21 days after administration of the immunoconjugates, followed by centrifugation (4° C., 10000×g, 3 min) to separate the serum. Total antibody concentration of each conjugates in serum was detected by ELISA and analyzed by Winnonlin 8.2 software.

Goat anti-human IgG Fc (Invitrogen, 31125) was coated on a 96-well micro plate (Thermo, cat:468667) in PBS with 2 micrograms/ml, using 100 microL per well, at 4° C. overnight. The next day the solution was removed and the plate was washed twice with 350 μL/well TBST. The plate was blocked by adding 200 microL/well of blocking buffer (3% BSA/TBST). The plate was placed at 37° C. for 2 h and washed twice with 350 μL/well TBST. A series of concentrations of standards and samples were added to each well, and the plate was placed at room temperature for 2 h. The solution was removed and the wells washed twice with 350 μL/well TBST. Goat Anti-human Kappa light chain (HRP) (abcam, ab202549) was diluted with the blocking buffer and added with 100 μL per well. The plate was incubated at room temperature for 1 h. Then the plate was washed four times with 350 μL/well TBST. 100 microL of TMB (solution A: solution B, 1:1) solution was added in each well and the plate was placed in the dark for 3-10 mins. 50 microL of stopping solution (2M H₂SO₄) was added and the optical density at 450 nm and 630 nm was read. Data was analyzed with GraphPadPrism5 software.

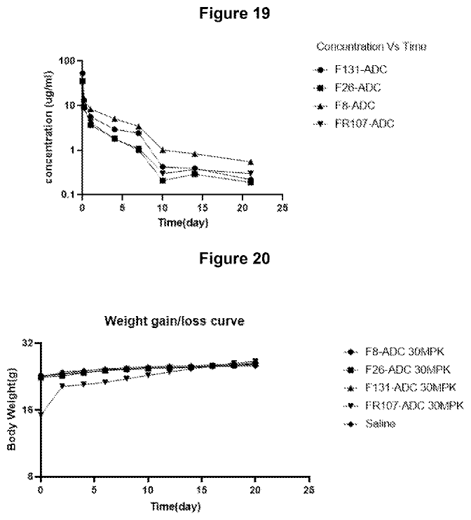

The result is shown in FIG. 19. FR107-ADC had higher serum clearance than F8-ADC and F131-ADC.

### 8.2 Safety Effect on Mice.

The mice used in the safety study is as descried before. 5 groups with 6 mice each group were treated with single dose of F8, F26, F131 and FR107 immunoconjugates intravenously (IV) at 30 mg/kg. Animals were checked daily for eating, drinking and activity, body weight gain/loss (body weight was measured once every two days), eye/hair matting and any other abnormal effects, death and observed clinical signs were recorded.

The body weight results are as shown in FIG. 20. The data demonstrated no significant gain or loss of weight in the treated mice.

### Example 9: Affinity Data of F131 to FOLR Family Proteins Tested by BLI

Recombinant proteins consisting of FOLR family proteins' extracellular domain linked to His tag were either purchased (from ACRO systems) or synthesized in house. For binding studies via biolayer interferometry (BLI), F131 (at 16.67 nM) was immobilized on anti-human IgG Fc biosensor tips (Fortebio). Binding assays using varying concentration (from 500 nM down to 7.8 nM) of recombinant antigen proteins in solution were performed using Octet RED (Fortebio). Association time was set at 180 s and dissociation time was set at 300 s. Binding affinity was calculated using ForteBio Data Acquisition 6.3 software (ForteBio), and affinity was derived by fitting the kinetic data to a 1:1 Langmuir binding model utilizing global fitting algorithms. F131 displayed high affinity to human FOLR1, while having low response to human FOLR2, and no response to human FOLR3, demonstrating binding specificity of F131 (Table 13). F131 demonstrated high bing affinity to human and cynomolgus monkey FOLR1 with an equilibrium dissociation constant (KD) of 1.5 and 8.1 nM, respectively. F131 displayed no cross-reactivity to rat FOLR1, and low cross-reactivity to mouse FOLR1 (KD=2.9 μM).

TABLE 13
Affinity data of F131 to FOLR family proteins tested by BLI

| | | Loading | | | | | | |
|---|---|---|---|---|---|---|---|---|
| | Loading | Conc. | Conc. | | | | | |
| Ag | Sample ID | (ug/ml) | (nM) | Response | KD (M) | ka (1/Ms) | kdis (1/s) | Full R\hat{ }2 |
| Hu−FOLR1 | F131 mAb | 2.5 | 500. | 0.1924 | 5.296E−09 | 2.261E05 | 1.198E−03 | 0.9978 |
| Hu−FOLR1 | F131 mAb | 2.5 | 250. | 0.1782 | 5.296E−09 | 2.261E05 | 1.198E−03 | 0.9978 |
| Hu−FOLR1 | F131 mAb | 2.5 | 125. | 0.1444 | 5.296E−09 | 2.261E05 | 1.198E−03 | 0.9978 |
| Hu−FOLR1 | F131 mAb | 2.5 | 62.5 | 0.1172 | 5.296E−09 | 2.261E05 | 1.198E−03 | 0.9978 |
| Hu−FOLR1 | F131 mAb | 2.5 | 31.3 | 0.0766 | 5.296E−09 | 2.261E05 | 1.198E−03 | 0.9978 |
| Hu−FOLR1 | F131 mAb | 2.5 | 15.6 | 0.0447 | 5.296E−09 | 2.261E05 | 1.198E−03 | 0.9978 |
| Hu−FOLR1 | F131 mAb | 2.5 | 7.82 | 0.0259 | 5.296E−09 | 2.261E05 | 1.198E−03 | 0.9978 |
| Hu−FOLR2 | F131 mAb | 2.5 | 500. | 0.037 | 1.420E−07 | 5.524E05 | 7.843E−02 | 0.9083 |
| Hu−FOLR2 | F131 mAb | 2.5 | 250. | 0.0207 | 1.420E−07 | 5.524E05 | 7.843E−02 | 0.9083 |
| Hu−FOLR2 | F131 mAb | 2.5 | 125. | 0.0188 | 1.420E−07 | 5.524E05 | 7.843E−02 | 0.9083 |
| Hu−FOLR2 | F131 mAb | 2.5 | 62.5 | 0.0356 | 1.420E−07 | 5.524E05 | 7.843E−02 | 0.9083 |
| Hu−FOLR2 | F131 mAb | 2.5 | 31.3 | *0.0068 | 1.420E−07 | 5.524E05 | 7.843E−02 | 0.9083 |
| Hu−FOLR2 | F131 mAb | 2.5 | 15.6 | *0.002 | 1.420E−07 | 5.524E05 | 7.843E−02 | 0.9083 |
| Hu−FOLR2 | F131 mAb | 2.5 | 7.82 | *0.0011 | 1.420E−07 | 5.524E05 | 7.843E−02 | 0.9083 |
| Hu−FOLR3 | F131 mAb | 2.5 | 500. | *0.0024 | 1.306E−04 | 1.663E03 | 2.173E−01 | 0.1554 |
| Hu−FOLR3 | F131 mAb | 2.5 | 250. | *0.0003 | 1.306E−04 | 1.663E03 | 2.173E−01 | 0.1554 |
| Hu−FOLR3 | F131 mAb | 2.5 | 125. | *0.0051 | 1.306E−04 | 1.663E03 | 2.173E−01 | 0.1554 |
| Hu−FOLR3 | F131 mAb | 2.5 | 62.5 | *−2.028E−03 | 1.306E−04 | 1.663E03 | 2.173E−01 | 0.1554 |
| Hu−FOLR3 | F131 mAb | 2.5 | 31.3 | *0.0028 | 1.306E−04 | 1.663E03 | 2.173E−01 | 0.1554 |
| Hu−FOLR3 | F131 mAb | 2.5 | 15.6 | *− 2.611E−03 | 1.306E−04 | 1.663E03 | 2.173E−01 | 0.1554 |
| Hu−FOLR3 | F131 mAb | 2.5 | 7.82 | *1.025E−03 | 1.306E−04 | 1.663E03 | 2.173E−01 | 0.1554 |

*Response below range of quantification

TABLE 14
Affinity data of F131 to species FOLR α proteins tested by BLI

| | | Loading | | | | | | |
|---|---|---|---|---|---|---|---|---|
| | Loading | Conc. | Conc. | | | | | |
| Ag | Sample ID | (ug/ml) | (nM) | Response | KD (M) | ka (1/Ms) | kdis (1/s) | Full R\hat{ }2 |
| Hu-Ag | F131 mAb | 2.5 | 500. | 0.181 | 1.528E−09 | 2.210E05 | 3.377E−04 | 0.9875 |
| Hu-Ag | F131 mAb | 2.5 | 250. | 0.1617 | 1.528E−09 | 2.210E05 | 3.377E−04 | 0.9875 |
| Hu-Ag | F131 mAb | 2.5 | 125. | 0.1458 | 1.528E−09 | 2.210E05 | 3.377E−04 | 0.9875 |
| Hu-Ag | F131 mAb | 2.5 | 62.5 | 0.1202 | 1.528E−09 | 2.210E05 | 3.377E−04 | 0.9875 |
| Hu-Ag | F131 mAb | 2.5 | 31.3 | 0.0869 | 1.528E−09 | 2.210E05 | 3.377E−04 | 0.9875 |
| Hu-Ag | F131 mAb | 2.5 | 15.6 | 0.0503 | 1.528E−09 | 2.210E05 | 3.377E−04 | 0.9875 |
| Hu-Ag | F131 mAb | 2.5 | 7.82 | 0.04 | 1.528E−09 | 2.210E05 | 3.377E−04 | 0.9875 |
| Cyno-Ag | F131 mAb | 2.5 | 500. | 0.2191 | 8.148E−09 | 1.929E05 | 1.572E−03 | 0.9864 |
| Cyno-Ag | F131 mAb | 2.5 | 250. | 0.209 | 8.148E−09 | 1.929E05 | 1.572E−03 | 0.9864 |
| Cyno-Ag | F131 mAb | 2.5 | 125. | 0.1775 | 8.148E−09 | 1.929E05 | 1.572E−03 | 0.9864 |
| Cyno-Ag | F131 mAb | 2.5 | 62.5 | 0.14 | 8.148E−09 | 1.929E05 | 1.572E−03 | 0.9864 |
| Cyno-Ag | F131 mAb | 2.5 | 31.3 | 0.084 | 8.148E−09 | 1.929E05 | 1.572E−03 | 0.9864 |
| Cyno-Ag | F131 mAb | 2.5 | 15.6 | 0.0485 | 8.148E−09 | 1.929E05 | 1.572E−03 | 0.9864 |
| Cyno−Ag | F131 mAb | 2.5 | 7.82 | 0.0537 | 8.148E−09 | 1.929E05 | 1.572E−03 | 0.9864 |
| Rat-Ag | F131 mAb | 2.5 | 500. | *0.0037 | 1.874E−04 | 5.494E03 | 1.030E00 | |
| Rat-Ag | F131 mAb | 2.5 | 250. | *−9.448E−04 | 1.874E−04 | 5.494E03 | 1.030E00 | |
| Rat-Ag | F131 mAb | 2.5 | 125. | *−4.774E−03 | 1.874E−04 | 5.494E03 | 1.030E00 | |
| Rat-Ag | F131 mAb | 2.5 | 62.5 | *−6.060E−03 | 1.874E−04 | 5.494E03 | 1.030E00 | |
| Rat-Ag | F131 mAb | 2.5 | 31.3 | *−5.315E−03 | 1.874E−04 | 5.494E03 | 1.030E00 | |
| Rat-Ag | F131 mAb | 2.5 | 15.6 | *−3.949E−03 | 1.874E−04 | 5.494E03 | 1.030E00 | |
| Rat-Ag | F131 mAb | 2.5 | 7.82 | *−5.629E−03 | 1.874E−04 | 5.494E03 | 1.030E00 | |
| Mouse-Ag | F131 mAb | 2.5 | 500. | 0.0167 | 2.957E−06 | 1.019E05 | 3.012E−01 | 0.6807 |
| Mouse-Ag | F131 mAb | 2.5 | 250. | *0.008 | 2.957E−06 | 1.019E05 | 3.012E−01 | 0.6807 |
| Mouse-Ag | F131 mAb | 2.5 | 125. | *−5.791E−04 | 2.957E−06 | 1.019E05 | 3.012E−01 | 0.6807 |
| Mouse-Ag | F131 mAb | 2.5 | 62.5 | *−1.782E−03 | 2.957E−06 | 1.019E05 | 3.012E−01 | 0.6807 |
| Mouse-Ag | F131 mAb | 2.5 | 31.3 | *−2.515E−03 | 2.957E−06 | 1.019E05 | 3.012E−01 | 0.6807 |
| Mouse-Ag | F131 mAb | 2.5 | 15.6 | *−5.099E−03 | 2.957E−06 | 1.019E05 | 3.012E−01 | 0.6807 |
| Mouse-Ag | \|F131 mAb | 2.5 | 7.82 | *0.0027 | 2.957E−06 | 1.019E05 | 3.012E−01 | 0.6807 |

*Response below range of quantification

### Example 10: F131 Binding Assay Data

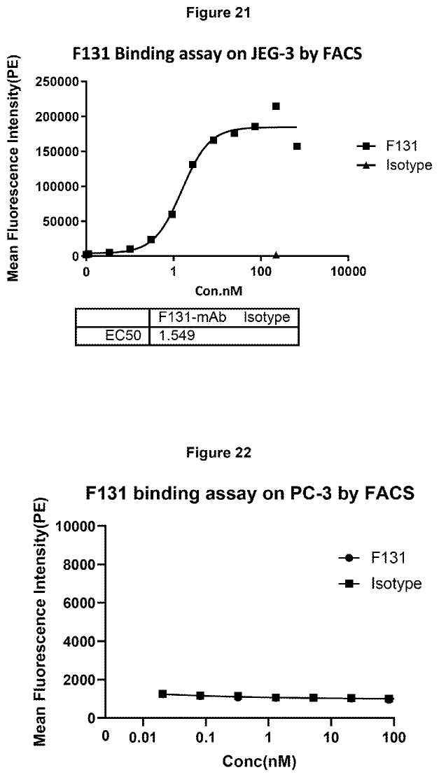

Binding activity of F131 was evaluated by flow cytometry (Beckman, Cytoflex) with cell lines that have either high (JEG-3) or no (PC-3) FOLR1 target expression. 3×10⁵ cells were seeded per well on a 96-well plate and incubated with 100 μl F131 in serial dilutions. After 30 min incubation at 4° C., cells were washed twice with PBS, stained with 100 μl of 1:200 diluted PE-conjugated anti human Fc in FACS buffer (1×PBS containing 1% BSA) and then incubated at 4° ° C. for 30 min. The cells were then washed two times with PBS and analyzed by flow cytometric analysis. F131 exhibited strong binding to human FOLR1-positive cell line, JEG-3 (FIG. 21), and no binding to human FOLR1-negative cell line, PC-3 (FIG. 22).

### Example 11: F131 Internalization in Tumor Cell Lines

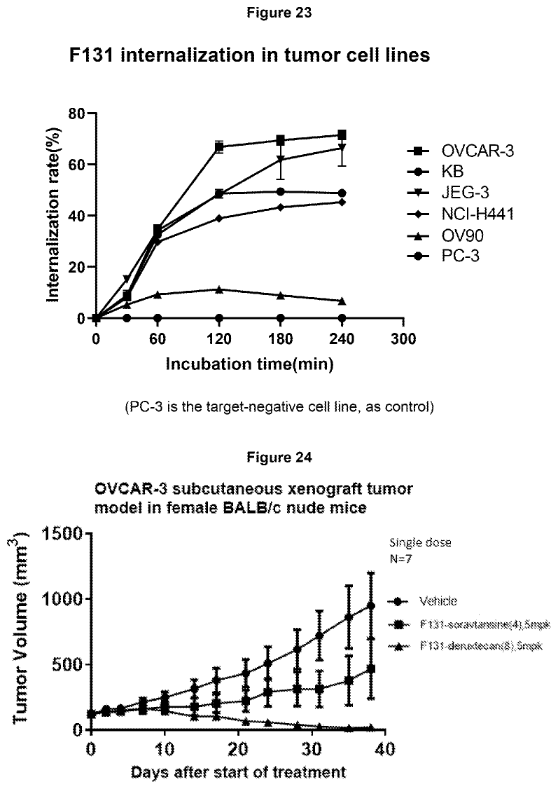

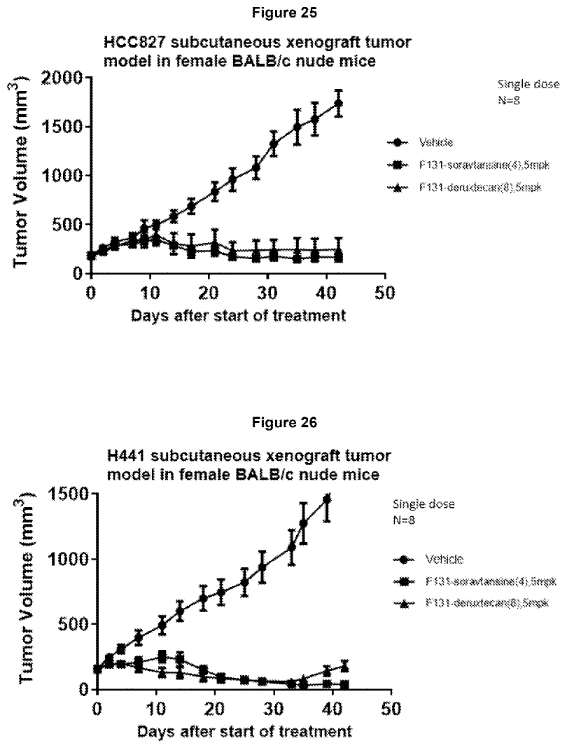

The internalization assay was conducted in time course. 3×10⁵ Cells were incubated for 30 min at 4° C. with 10 μg/ml of F131 in FACS buffer (1×PBS containing 0.1% BSA). Cells were washed at 4° C. to remove unbound material and kept on ice or shifted to 37° C. as needed. At progressive time points (0 h, 0.5 h, 1 h, 2 h, 3 h, 4 h), cells were stained with PE-conjugated anti-human Fc for 30 min at 4° C. and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time 0, then divided by the MFI of cell surface-bound antibody at 4° C. at time 0. F131 displayed rapid internalization on FOLR1-expressing cell lines (OVCAR-3, KB, JEG-3, NCI-H₄₄₁, OV90), while no internalization on FOLR1 non-expressing cells (PC-3) (FIG. 23).

### Example 12: In Vivo Efficacy of the F131 Conjugates

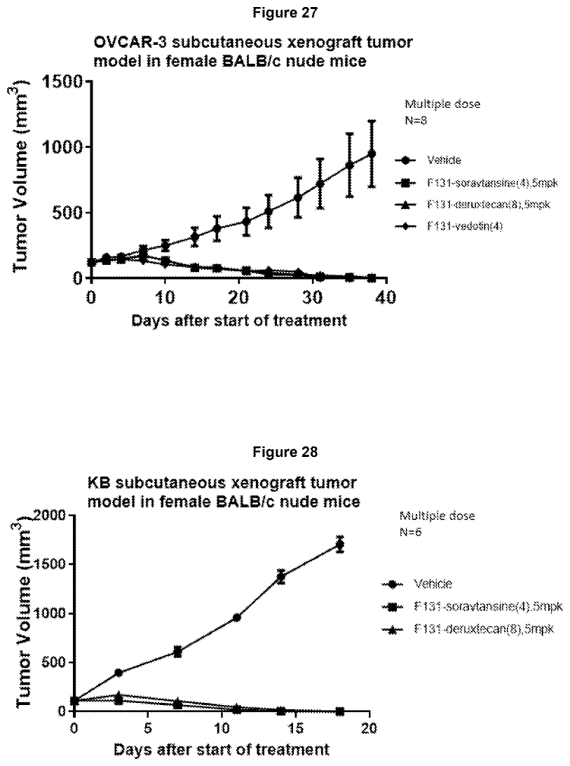

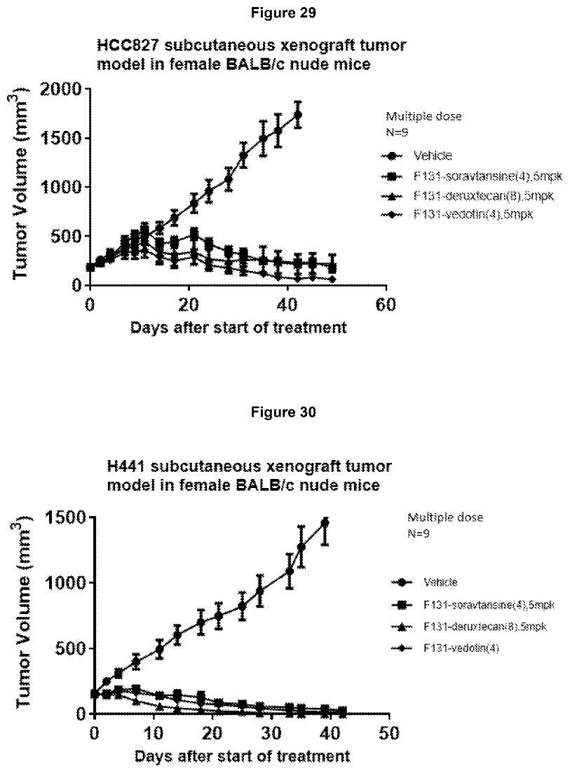

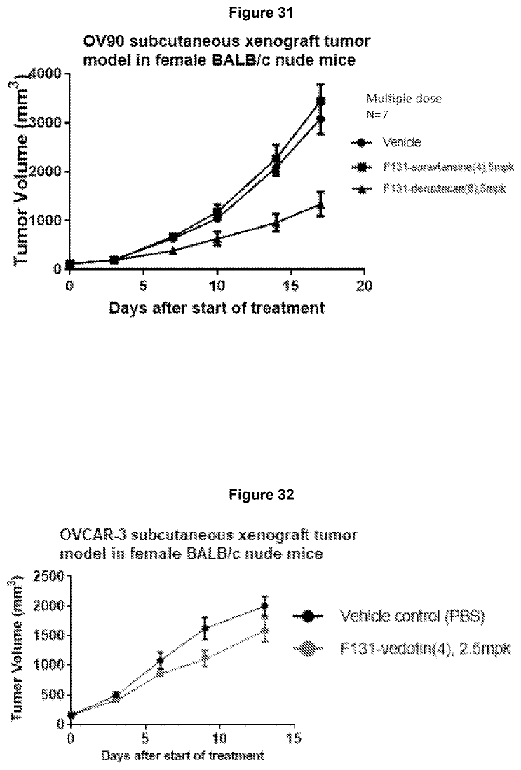

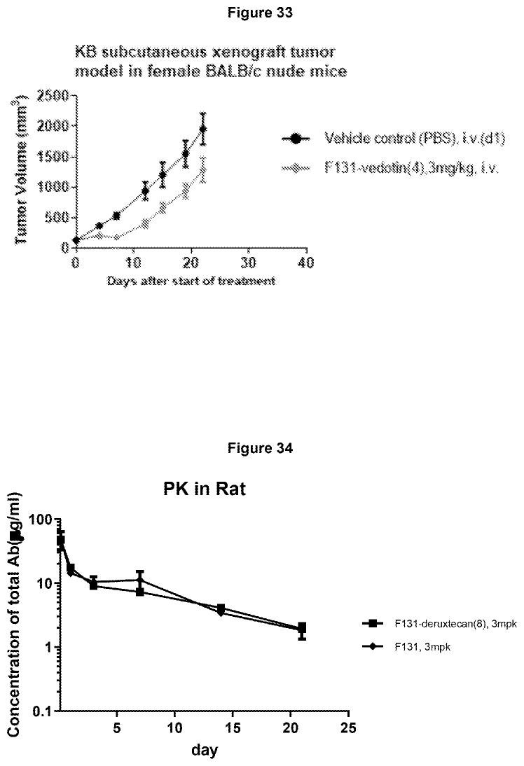

Antitumor activity of F131 in conjugate with various benchmarking linker-drugs (Table 15) was evaluated in the cell line-derived xenograft (CDX) models. For preparation of F131-soravtansine: The solution of sulfo-SPDB-DM4 (10 mg/mL in DMSO) was added to the solution of 2 mL of antibody (10 mg/mL in 50 mM phosphate buffer containing 5 mM EDTA pH7.4), the molar ratio of sulfo-SPDB-DM4 to mAb is 6.0. Conduct the reaction for 6 hours at 25° C. The excess sulfo-SPDB-DM4 and its impurities were removed by ultrafiltration with 50 mM sodium phosphate buffer. The ADC was stored in 20 mM histidine buffer containing 6% sucrose and 0.02% (w/V) Tween 20 by UFDF. The purity of SEC-HPLC was 97.9% and DAR value was 3.5 based on LC-MS. For preparation of F131-deruxtecan, 2 mL of antibody (10 mg/mL) in 50 mM sodium phosphate buffer containing 5 mM EDTA (pH=6.9) was added the aqueous of 10 mM TCEP HCl (Tris(2-carboxyethyl) phosphine HCl), the molar ratio of TCEP to mAb is 8.0. Conduct the reducing reaction for 2 hours at 25° C. Dissolve deruxtecan in DMSO at a concentration of 20 mg/mL and add it to reduced antibody at a molar ratio of 12 (deruxtecan/mAb). The coupling reaction is stirred for 8 hours at 25° C. The excess deruxtecan and its impurities were removed by ultrafiltration with 50 mM sodium phosphate buffer. The ADC was stored in 20 mM histidine buffer containing 6% sucrose and 0.02% (w/V) Tween 20 by UFDF. The purity of SEC-HPLC was 97.5% and DAR value was 7.7 based on LC-MS. For preparation of F131-vedotin, 2 mL of antibody (10 mg/mL) in 50 mM sodium phosphate buffer containing 5 mM EDTA (pH=6.9) was added the aqueous of 10 mM TCEP HCl (Tris(2-carboxyethyl) phosphine HCl), the molar ratio of TCEP to mAb is 2.2. Conduct the reducing reaction for 2 hours at 25° C. Dissolve vedotin in DMSO at a concentration of 20 mg/mL and add it to reduced antibody at a molar ratio of 5.0 (vedotin/mAb). The coupling reaction is stirred for 2 hours at 25° C. The excess vedotin and its impurities were removed by ultrafiltration with 50 mM sodium phosphate buffer. The ADC was stored in 20 mM histidine buffer containing 6% sucrose and 0.02% (w/V) Tween 20 by UFDF. The purity of SEC-HPLC was 97.5% and DAR value was 3.9 based on HIC-HPLC. For characterization of target (FOLR1) copy number (binding sites) per cell (Table 15): assay was carried out per instructions in the QIFIKIT (DAKO, K0078) assay kit. Briefly, cells were labeled with primary mouse monoclonal antibody against human FOLR1. Cells, Set-up beads, and Calibration beads were then labeled in parallel with the fluorescein-conjugated anti-mouse secondary antibody. Samples were analyzed on flow cytometry and copy number was calculated based on the calibration curve. For the CDX studies with the F131 conjugates: appropriate amount of cells suspended in either Matrigel/medium (1:1), or medium, subcutaneously, into female BALB/c nude mice. At day 6-26 after tumor inoculation, mice with average tumor size of 110-180 mm³ were selected and assigned into treatment groups (n=6-9 per group) using stratified randomization based off their tumor volumes. Treatment with intravenous injection of the F131 conjugates or vehicle control imitated on day 1 after randomization and was either in the single-dose (on day 1, FIG. 24, 25, 26, 32, 33) model, or multiple-dose (on day 1, 4, 8, 11) model (FIG. 27, 28, 29, 30, 31). Tumor size was measured twice a week using standard methods. Animal body weight was monitored as an indirect measure of toxicity. No morbidity or deaths were observed in any of the treatment groups during the treatment duration. Compared to vehicle control, the F131 conjugates conferred substantial tumor growth inhibition in all models tested.

TABLE 15

| Benchmarking | |
|---|---|
| ADC (DAR) | Linker-drug |
| F131-soravtansine(4) | SPDB-DM4 |
| F131-deruxtecan(8) | mc-GGFG-DXd |
| F131-vedotin(4) | mc-vc-PAB-MMAE |

| Cell line | Tumor type | Copy number |
|---|---|---|
| OVCAR-3 | ovarian | 290291 |
| KB | oral epithelial | 341364 |
| HCC827 | NSCLC | 40919 |
| NCI-H441 | NSCLC | 46889 |
| OV90 | ovarian | 380 |

### Example 13: PK Study in Rat Model of F131 and Conjugates

F131 and its conjugates were intravenously administered at a single dose of 3 mg/kg to male sprague dawley rats (n=3 per group). Orbital blood was sampled from each rat at various time points post dosing. Total Ab concentration (in detecting F131 and its conjugate in plasma) were analyzed by an ELISA kit (Genscript) and calculated using the Winnonlin 8.2 software. F131-deruxtecan exhibited excellent PK in rat that is indistinguishable from the parental mAb (FIG. 34). F131-vedotin exhibited stable PK in rat although clearance appears to be somewhat faster than the parental mAb (FIG. 35).

### Example 14: F131-Deruxtecan PK and Tolerability in the Pilot Cynomolgus Toxicity Study

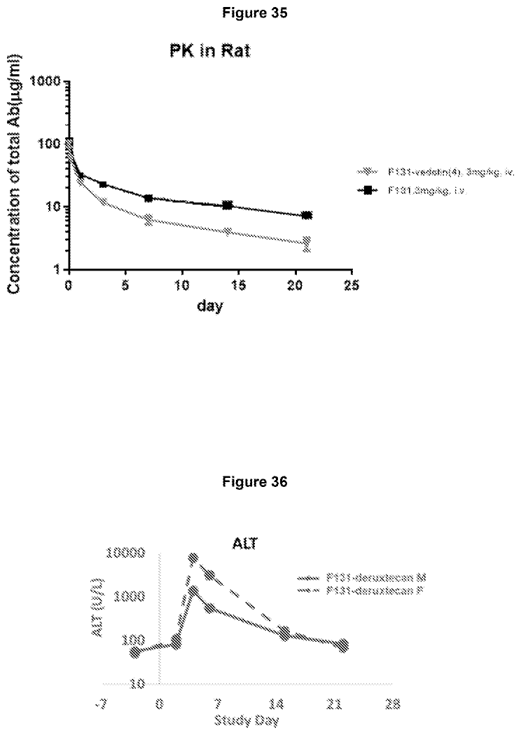

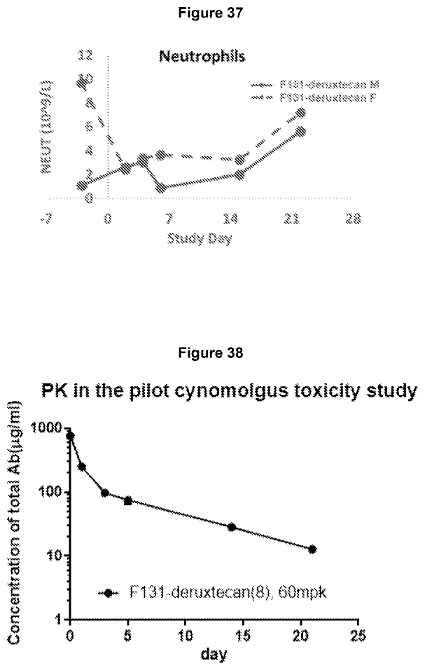

F131-deruxtecan was intravenously administered at a single dose of 60 mg/kg to one male and one female cynomolgus monkeys on day 1. Clinical signs, body weight, food consumption, and clinical pathology were monitored throughout the study. Necropsy was scheduled on day 22. Toxicokinetic samples were collected from each animal at 0, 24, 72, 120, 336, and 504 hours after completion of the administration. Total Ab concentrations in representing F131 and F131-conjugate in plasma were analyzed by an ELISA kit (from Genscript) and calculated using Winnonlin 8.2 software. Both animals survived until scheduled necropsy. Clinical observations, hematology, and clinical chemistry are shown in Table 16 and FIGS. 36 and 37. All changes displayed a trend of recovery by Day 22. No toxicological abnormality was noted in body weight, body temperature, coagulation, urinalysis, or gross necropsy. F131-deruxtecan exhibited stable pharmacokinetic characteristics in cynomolgus monkey plasma (FIG. 38).

TABLE 16

| | Clinical | | Clinical |
|---|---|---|---|
| Test Article | Observations | Hematology | Chemistry |
| F131- | Skin cold to | ↓: WBC; NEUT/ | ↑↑↑: ALT; AST |
| deruxtecan | touch | NEUT %; LYM; | ↓: TG |
| (60 mg/kg) | Drinking | MONO; RET/ | |
| (one male; | decreased | RET %; | |
| one female) | Feces soft | | |
| | Appetite | | |
| | lessens | | |

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications, including patents, patent application publications, and scientific literature, are cited herein, the disclosures of which are incorporated by reference in their entireties for all purposes.

1.-72. (canceled)

73. A binding agent comprising:
a heavy chain variable (VH) region and a light chain variable (VL) region, the VH region comprising complementarity determining regions HCDR1, HCDR2 and HCDR3 and the VL region comprising LCDR1, LCDR and, the VH and VL CDRs having amino acids sequences selected from the sets of amino acid sequences that are selected from:
a. SEQ ID NO:25, SEQ ID NO:26, SEQ ID NO:27, SEQ ID NO:28, SEQ ID NO:29 and SEQ ID NO:30, respectively; and
b. SEQ ID NO:31, SEQ ID NO:26, SEQ ID NO:32, SEQ ID NO:33, SEQ ID NO:34 and SEQ ID NO:35, respectively.

74. The binding agent of claim 73, wherein (a) the HCDR1 comprises the amino acid sequence set forth in SEQ ID NO: 25, (b) the HCDR2 comprises the amino acid sequence set forth in SEQ ID NO: 26, (c) the HCDR3 comprises the amino acid sequence set forth in SEQ ID NO: 27, (d) the LCDR1 comprises the amino acid sequence set forth in SEQ ID NO: 28, (e) the LCDR2 comprises the amino acid sequence set forth in SEQ ID NO: 29, and (f) the LCDR3 comprises the amino acid sequence set forth in SEQ ID NO: 30.

75. The binding agent of claim 73, wherein the VH and VL regions have amino acid sequences that are selected from:
a. VH: SEQ ID NO: 1 and VL: SEQ ID NO:2;
b. VH: SEQ ID NO:3 and VL: SEQ ID NO:4;
c. VH: SEQ ID NO:5 and VL: SEQ ID NO:6;
d. VH: SEQ ID NO:7 and VL: SEQ ID NO:8;
e. VH: SEQ ID NO:9 and VL: SEQ ID NO:10;
f. VH: SEQ ID NO:11 and VL: SEQ ID NO:12;
g. VH: SEQ ID NO:13 and VL: SEQ ID NO:14;
h. VH: SEQ ID NO: 15 and VL: SEQ ID NO: 16;
i. VH: SEQ ID NO: 17 and VL: SEQ ID NO:18;
j. VH: SEQ ID NO: 19 and VL: SEQ ID NO:20;
k. VH: SEQ ID NO:21 and VL: SEQ ID NO:22; and
l. VH: SEQ ID NO:23 and VL: SEQ ID NO:24, respectively.
wherein the VH and VL regions comprise framework regions that are optionally modified with from 1 to 8 amino acid substitutions, deletions or insertions.

76. The binding agent of claim 75, wherein the VH and VL regions comprise framework regions that are optionally modified with from 1 to 4 amino acid substitutions, deletions or insertions.

77. The binding agent of claim 73, wherein the VH and VL regions have amino acid sequences that are selected from:
a. VH: SEQ ID NO: 1 and VL: SEQ ID NO:2;
b. VH: SEQ ID NO:3 and VL: SEQ ID NO:4;
c. VH: SEQ ID NO:5 and VL: SEQ ID NO:6;
d. VH: SEQ ID NO:7 and VL: SEQ ID NO:8;
e. VH: SEQ ID NO:9 and VL: SEQ ID NO:10;
f. VH: SEQ ID NO: 11 and VL: SEQ ID NO:12;
g. VH: SEQ ID NO: 13 and VL: SEQ ID NO: 14;
h. VH: SEQ ID NO:15 and VL: SEQ ID NO:16;
i. VH: SEQ ID NO:17 and VL: SEQ ID NO:18;
j. VH: SEQ ID NO: 19 and VL: SEQ ID NO:20;
k. VH: SEQ ID NO:21 and VL: SEQ ID NO:22; and
l. VH: SEQ ID NO:23 and VL: SEQ ID NO:24, respectively.

78. The binding agent of claim 77, wherein the VH and VL regions have amino acid sequences that are selected from:
a. VH: SEQ ID NO:3 and VL: SEQ ID NO:4;
b. VH: SEQ ID NO:7 and VL: SEQ ID NO:8;
c. VH: SEQ ID NO:9 and VL: SEQ ID NO:10;
d. VH: SEQ ID NO: 11 and VL: SEQ ID NO: 12;
e. VH: SEQ ID NO:15 and VL: SEQ ID NO: 16;
f. VH: SEQ ID NO: 17 and VL: SEQ ID NO:18;
g. VH: SEQ ID NO: 19 and VL: SEQ ID NO:20; and
h. VH: SEQ ID NO:21 and VL: SEQ ID NO:22, respectively.

79. The binding agent of claim 78, wherein the VH and VL regions have amino acid sequences that are selected from:
a. VH: SEQ ID NO:3 and VL: SEQ ID NO:4;
b. VH: SEQ ID NO:7 and VL: SEQ ID NO:8; and
c. VH: SEQ ID NO:21 and VL: SEQ ID NO:22, respectively.

80. The binding agent of claim 79, wherein the VH has the amino acid sequence set forth in SEQ ID NO: 21 and the VL has the amino acid sequence set forth in SEQ ID NO: 22.

81. The binding agent of claim 73, wherein the VH and the VL regions comprise human framework regions.

82. The binding agent of claim 73, wherein the binding agent is an antibody or antigen-binding portion thereof.

83. The binding agent of claim 82, wherein the binding agent is a monoclonal antibody, a Fab, a Fab′, an F(ab′), an Fv, a scFv, a single domain antibody, a diabody, a bi-specific antibody, or a multi-specific antibody.

84. The binding agent of claim 83, wherein the binding agent further comprises a heavy chain constant region.

85. The binding agent of claim 84, wherein the heavy chain constant region is of the IgG isotype.

86. The binding agent of claim 85, wherein the heavy chain constant region is an IgG1 constant region.

87. The binding agent of claim 86, wherein the IgG1 constant region has the amino acid sequence set forth in SEQ ID NO:39.

88. The binding agent of claim 73, wherein the binding agent further comprises a light chain constant region.

89. The binding agent of claim 88, wherein the light chain constant region is of the kappa isotype.

90. The binding agent of claim 89, wherein the kappa isotype has the amino acid sequence set forth in SEQ ID NO:40.

91. The binding agent of claim 90, the heavy chain constant region further comprises L234A and L235A mutations, according to the EU numbering of Kabat.

92. A nucleic acid encoding the binding agent of claim 73.

93. A vector comprising the nucleic acid of claim 92.

94. A cell line comprising the nucleic acid of claim 92.

95. A conjugate comprising:
the binding agent of claim 73,
at least one linker attached to the binding agent; and
at least one drug attached to each linker.

96. The conjugate of claim 95, wherein each linker is attached to the binding agent via an interchain disulfide residue, a lysine residue, an engineered cysteine residue, a glycan, a modified glycan, an N-terminal residue of the binding agent or a polyhistidine peptide attached to the binding agent.

97. The conjugate of claim 95, wherein the average drug loading of the conjugate is from about 1 to about 16.

98. The conjugate of claim 97, wherein the average drug loading of the conjugate is from about 1 to about 8.

99. The conjugate of claim 98, wherein the average drug loading of the conjugate is 4 or 8.

100. The conjugate of claim 95, wherein each drug is selected from a cytotoxic agent, an immunomodulatory agent, a nucleic acid, a growth inhibitory agent, a PROTAC, a toxin and a radioactive isotope.

101. The conjugate of claim 100, wherein the drug is a cytotoxic agent.

102. The conjugate of claim 95, wherein the linker comprises mc-VC-PAB, CL2, CL2A or (Succinimid-3-yl-N)—(CH₂)ₙ—C(═O)-Gly-Gly-Phe-Gly-NH—CH₂—O—CH₂—(C═O)—, wherein n=1 to 5.

103. A pharmaceutical composition comprising the binding agent of claim 73 and a pharmaceutically acceptable carrier.

104. A pharmaceutical composition comprising the conjugate of claim 95 and a pharmaceutically acceptable carrier.

105. A method of treating a FOLR1+ cancer, comprising administering to a subject in need thereof a therapeutically effective amount of the pharmaceutical composition of claim 103.

106. A method of treating a FOLR1+ cancer, comprising administering to a subject in need thereof a therapeutically effective amount of the pharmaceutical composition of claim 104.
